(19) United States
(12) Patent Application Publication
(10) Pub. No.: US 2024/0350136 A1
(43) Pub. Date: Oct. 24, 2024

(54) VESSEL CLOSURE DEVICE WITH IMPROVED SAFETY AND TRACT HEMOSTASIS

(71) Applicant: Abbott Cardiovascular Systems, Inc., Santa Clara, CA (US)

(72) Inventors: Timothy C. Reynolds, Sunnyvale, CA (US); Austin R. Gipe, Danville, CA (US); Khanh Duong, San Jose, CA (US); Michael L. Green, Pleasanton, CA (US); Aaron M. Fortson, Sebastopol, CA (US)

(21) Appl. No.: 18/630,600

(22) Filed: Apr. 9, 2024

Related U.S. Application Data
(60) Provisional application No. 63/495,360, filed on Apr. 11, 2023.

(51) Int. Cl. A61B 17/06; A61B 17/00; A61B 17/04; A61B 17/12; A61L 24/00; A61L 24/06

(52) U.S. Cl. CPC A61B 17/06166; A61L 24/0031; A61L 24/06; A61B 2017/00004; A61B 2017/00473; A61B 2017/00606; A61B 2017/00623; A61B 2017/00663; A61B 2017/00862; A61B 2017/0404; A61B 2017/0409; A61B 2017/12004

(57) ABSTRACT

A vessel closure device for delivering substantially immediate hemostasis at a puncture site in a wall of a blood vessel includes an intravascular anchor having one or more suture attachment points, an extravascular cap having a lumen, a sealant, and a suture connected to at least one of the one or more suture attachment points of the intravascular anchor and threaded through the lumen of the extravascular cap, wherein each of the intravascular anchor, extravascular cap, sealant, and suture are formed of bioabsorbable materials. Delivery systems for delivering such a vessel closure device are also disclosed.

### CROSS-REFERENCE TO RELATED APPLICATIONS

This application claims the benefit of and priority to U.S. Provisional Patent Application Ser. No. 63/495,360 filed on Apr. 11, 2023, and entitled “VESSEL CLOSURE DEVICE WITH IMPROVED SAFETY AND TRACT HEMOSTASIS,” which application is expressly incorporated herein by reference in its entirety.

### BACKGROUND

### 1. The Field of the Invention

The present disclosure relates generally to systems, devices, and methods for blocking an opening in body lumens. More particularly, the present disclosure relates to techniques for percutaneous closure of arterial and venous puncture sites, which are usually accessed through a tissue tract.

### 2. The Relevant Technology

A number of diagnostic and interventional vascular procedures are now performed translumenally. A catheter is introduced to the vascular system at a convenient access location and guided through the vascular system to a target location using established techniques. Such procedures require vascular access, which is usually established during the well-known Seldinger technique. Vascular access is generally provided through an introducer sheath, which is positioned to extend from outside the patient body into the vascular lumen. When vascular access is no longer required, the introducer sheath is removed and bleeding at the puncture site stopped.

One common approach for providing hemostasis (the cessation of bleeding) is to apply external force near and upstream from the puncture site, typically by manual compression. This approach suffers from a number of disadvantages. For example, the manual compression procedure is time consuming, frequently requiring one-half hour or more of compression before hemostasis is achieved. Additionally, such compression techniques rely on clot formation, which can be delayed until anticoagulants used in vascular therapy procedures (such as for heart attacks, stent deployment, non-optical PTCA results, and the like) wear off. The anticoagulants may take two to four hours to wear off, thereby increasing the time required before completion of the manual compression procedure.

The manual compression procedure is uncomfortable for the patient and frequently requires analgesics to be tolerable. Moreover, the application of excessive pressure can at times totally occlude the underlying blood vessel, resulting in ischemia and/or thrombosis. Following manual compression, the patient typically remains recumbent from four to as much as twelve hours or more under close observation to assure continued hemostasis. During this time, renewed bleeding may occur, resulting in blood loss through the tract, hematoma and/or pseudo-aneurysm formation, as well as arteriovenous fistula formation. These complications may require blood transfusion and/or surgical intervention.

The incidence of complications from the manual compression procedure increases when the size of the introducer sheath grows larger, and/or when the patient is anticoagulated. The compression technique for arterial closure can be risky, and is expensive and onerous to the patient. Although the risk of complications can be reduced by using highly trained individuals, dedicating such personnel to this task is both expensive and inefficient. Nonetheless, as the number and efficacy of translumenally performed diagnostic and interventional vascular procedures increases, the number of patients requiring effective hemostasis for a vascular puncture continues to increase.

Vascular closure devices have been introduced to reduce the time to hemostasis, enable early ambulation and improve patient comfort. Initially, devices focused on technologies involving a suture or collagen plug. These technologies close the hole or puncture site, however, they often leave an intravascular component in the vessel which can cause complications and result in residual bleeding or tract ooze. Some amount of slow and steady tract bleeding is a common occurrence. This bleeding usually requires direct management by a trained health care professional until it is completely stopped. Anticoagulant medications typically given to catheterized patients can exacerbate bleeding and may require management with manual compression until the medication wears off.

### BRIEF SUMMARY

This application is directed to a vessel closure device for delivering rapid hemostasis at a puncture site in a wall of a blood vessel. The vessel closure device can include an intravascular anchor having one or more suture attachment points, an extravascular cap having a lumen, a sealant, and a suture connected to at least one of the one or more suture attachment points of the intravascular anchor and threaded through the lumen of the extravascular cap. Each of the intravascular anchor, extravascular cap, sealant, and suture can be formed of bioabsorbable materials.

The present disclosure relates to a vessel closure device for delivering substantially immediate hemostasis at a puncture site in a wall of a blood vessel, the closure device includes an intravascular anchor comprising one or more suture attachment points, an extravascular cap having a lumen, a sealant, and a suture connected to at least one of the one or more suture attachment points of the intravascular anchor and threaded through the lumen of the extravascular cap and through the sealant to connect the intravascular anchor to the extravascular cap and to the sealant. Each of the intravascular anchor, extravascular cap, sealant, and suture are formed of bioabsorbable materials.

The present disclosure also relates to a vessel closure device having one or more of an elongate body having a flexible member and a keel. The elongate body may optionally include a plurality of ribs radiating from the keel to a raised edge of the elongate body. The closure device also includes an extravascular cap being formed of an elastomeric material, a sealant being formed of polyethylene glycol (PEG), a suture having a distal suture portion and a proximal suture portion, where a diameter of the lumen of the extravascular cap is smaller than a diameter of the distal suture portion, and an intravascular anchor.

In some embodiments, the closure device includes an intravascular anchor having one or more suture attachment points; an extravascular cap having a lumen; a sealant having a lumen; and a suture connected to at least one of the one or more suture attachment points of the intravascular anchor, threaded through the lumen of the extravascular cap, and threaded through the lumen of the sealant to connect the intravascular anchor to the extravascular cap and the sealant. The suture can include a proximal suture portion and a distal suture portion, wherein the distal suture portion has a diameter greater than a diameter of the lumen of the extravascular cap. The distal suture portion can create an interference fit to lock the extravascular cap over the puncture site, and each of the intravascular anchor, extravascular cap, sealant, and suture are formed of bioabsorbable materials.

The present disclosure also relates to a vessel closure device having one or more of: an extravascular cap formed of flexible material; a suture being a braided suture; a sealant being threaded onto the suture at a location proximal to the extravascular cap; the sealant, when activated, locking the extravascular cap in place and coagulating an access tract of the puncture site, providing substantially immediate hemostasis; the intravascular anchor having an elongate body; a raised keel located on a central axis of the elongate body and spanning the length of the elongate body (optionally including one or more suture attachment points); and the sealant being formed of polyethylene glycol (PEG).

The present disclosure also relates to a delivery system for delivering a vessel closure device to provide substantially immediate hemostasis at a puncture site in a blood vessel wall. The delivery system can include a handle assembly, a delivery assembly, and a vessel closure device. The handle and delivery assemblies are configured to removably engage with each other to deliver the vessel closure device to the puncture site.

The present disclosure also relates to an intravascular anchor for a vessel closure device for delivering substantially immediate hemostasis at a puncture site in a wall of a blood vessel. In some embodiments, the intravascular anchor includes an elongate body comprising a flexible membrane for conforming to the wall of the blood vessel, a keel having one or more suture attachment points, wherein the keel is an elongate member centrally located along a central axis of the elongate body, and wherein the intravascular anchor comprises a bioabsorbable material.

These and other objects and features of the present disclosure will become more fully apparent from the following description and appended claims, or may be learned by the practice of the invention as set form hereinafter.

### BRIEF DESCRIPTION OF THE DRAWINGS

A description of various aspects and features of the disclosure will be rendered by reference to various representative embodiments thereof illustrated in the appended drawings. It is appreciated that these drawings depict only typical embodiments of the invention and are therefore not to be considered limiting of its scope.

FIGS. 1A-1C illustrate a delivery system in which a closure device can be implemented according to one example.

FIG. 1D illustrates an alternate delivery system for deploying the closure device according to the present disclosure.

FIG. 1E illustrates a partial cross-sectional view of the alternate delivery system of FIG. 1D.

FIG. 1F illustrates a schematic representation of another alternate delivery system according to the present disclosure.

FIGS. 2A and 2B illustrate example embodiments of a closure device.

FIG. 3A illustrates an embodiment of a cap of a closure device.

FIG. 3B illustrates a cross-sectional view of the cap of FIG. 3A.

FIG. 3C illustrates a cap similar to that of FIG. 3A with an adhesive layer.

FIG. 3D illustrates a cross-sectional view of the cap of FIG. 3C.

FIG. 4 illustrates a cross-sectional view of a closure device as applied to a vessel.

FIG. 5 illustrates a cross-sectional view of a closure device as applied to a vessel through an access tract.

FIGS. 6A and 6B illustrate cross-sectional views of a closure device as applied to a vessel through an access tract.

FIGS. 7A-7D illustrate an embodiment of an intravascular anchor of a closure device.

FIGS. 7E and 7F illustrate an alternate embodiment of an intravascular anchor of a closure device.

FIGS. 7G and 7H illustrate an alternate embodiment of a closure device.

FIG. 8A illustrates a lumen-facing side of an alternate embodiment of an intravascular anchor.

FIG. 8B illustrates an intima-facing side of the embodiment of the intravascular anchor of FIG. 8A.

FIG. 9A illustrates a lumen-facing side of another embodiment of an intravascular anchor.

FIG. 9B illustrates an intima-facing side of the embodiment of the intravascular anchor of FIG. 9A.

FIG. 10A illustrates a lumen-facing side of another embodiment of an intravascular anchor.

FIG. 10B illustrates an intima-facing side of the embodiment of the intravascular anchor of FIG. 10A.

FIGS. 11A-11D illustrate a method of delivering a closure device to an access site on a vessel.

FIG. 12 illustrates an alternate embodiment of a delivery system for a closure device.

FIGS. 13A and 13B illustrate side views of a handle assembly of the delivery system of FIG. 12.

FIG. 13C illustrates a perspective view of the handle assembly of FIGS. 13A and 13B.

FIG. 13D illustrates a top plan view of the handle assembly of FIGS. 13A-13C.

FIG. 13E illustrates a cross-sectional view of the handle assembly of FIGS. 13A-13D.

FIG. 13F illustrates an enlarged view of the handle assembly region 13F shown in FIG. 13E.

FIG. 14A illustrates an exploded view of the handle assembly of FIG. 13C of the delivery system.

FIG. 14B illustrates an enlarged view of a chamber of the handle assembly of FIGS. 12-14A.

FIG. 14C illustrates a cross-sectioned perspective view of the handle assembly of FIGS. 13A-13E with an implant assembly removed from the handle assembly.

FIG. 14D illustrates a cross-sectional view of a slider of the implant assembly of FIG. 14C.

FIGS. 14E and 14F illustrate perspective views of the slider of FIG. 14D as positioned within a handle body.

FIG. 15 illustrates a close-up of the implant assembly of FIGS. 14A and 14C.

FIG. 16 illustrates an exploded view of the implant assembly of FIG. 14C.

FIGS. 17A and 17B illustrate a dilator tube and dilator hub for implantation of a closure device.

FIGS. 18A and 18B illustrate a delivery sheath and sheath hub of a delivery system.

FIGS. 19A and 19B illustrate the insertion and attachment of a handle assembly to a delivery sheath and sheath hub.

FIG. 19C illustrates the delivery system of FIG. 19A in a partially-deployed state.

FIG. 19D illustrates a close-up view of the implant assembly partially deployed from the delivery sheath as shown in FIG. 19C.

FIGS. 20A-20B illustrate a dilator tube and dilator hub being inserted into a delivery sheath and sheath hub according to a method of delivering a closure device to an access site on a vessel.

FIG. 21A illustrates the combination dilator tube and delivery sheath being inserted through a tissue tract according to according to a method of delivering a closure device to an access site on a vessel.

FIG. 21B illustrates the delivery sheath in the tissue tract according to a method of delivering a closure device to an access site on a vessel.

FIGS. 21C-21D illustrates the handle assembly being connected to the delivery sheath and sheath hub according to a method of delivering a closure device to an access site on a vessel.

FIG. 22 illustrates partial deployment of the closure device according to a method of delivering a closure device to an access site on a vessel.

FIGS. 23A-23C illustrate deployment of the closure device, which is followed by removal of the handle assembly and delivery sheath according to a method of delivering a closure device to an access site on a vessel.

FIGS. 24A-24Q illustrate an embodiment of a latch or interlock assembly used in conjunction with a handle assembly for delivering a closure device to an access site on a vessel.

FIGS. 25A-25C illustrate a bottom view of the latch assembly of FIGS. 24A-24Q.

FIGS. 26A-26F illustrate another embodiment of a latch or interlock assembly used in conjunction with a handle assembly for delivering a closure device to an access site on a vessel.

FIGS. 27A-27F illustrate yet another embodiment of a latch or interlock assembly used in conjunction with a handle assembly for delivering a closure device to an access site on a vessel.

FIGS. 28A-28D illustrate an alternative embodiment of an external actuator or slider mechanism that can be used as part of a handle assembly for delivering a closure device to an access site on a vessel.

FIGS. 29A-29B illustrate perspective and exploded views, respectively, of an alternative embodiment of a delivery system including dual sliders.

FIG. 29C illustrates a cross-sectional view through the chamber assembly of the delivery system shown in FIG. 29B.

FIG. 29D illustrates a perspective view of the delivery system of FIG. 29A-29B, once the dilator assembly has been removed, and the sheath hub is coupled to the housing.

FIGS. 30A-30B illustrate perspective and exploded views, respectively, of an alternative embodiment of a delivery system configured with a single slider.

FIG. 30C illustrates a perspective view of the delivery system of FIG. 30A-30B, once the dilator assembly has been removed, and the sheath hub is coupled to the housing.

FIGS. 31A-31B illustrate perspective and exploded views, respectively, of an alternative embodiment of a delivery system configured with a single slider.

FIG. 31C illustrates a perspective view of the delivery system of FIG. 31A-31B, once the dilator assembly has been removed, and the sheath hub is coupled to the housing.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to apparatuses, systems, and methods to deliver a closure device or closure implant configured to close an opening (e.g., a puncture in a blood vessel) formed in tissue. The closure devices or closure implants are configured to provide immediate or substantially immediate hemostasis at the opening. Immediate or substantially homeostasis can be achieved via “sandwiching” the closure device or closure implant around the opening. The disclosed closure devices or implants are also configured to deliver a hemostatic agent or sealant in an access tract within the tissue to eliminate or reduce track ooze. The configuration of the disclosed closure devices or closure implants can prevent extravascular components from passing through the puncture site, as the disclosed closure devices or closure implants also have improved resistance to fracture and possible embolization.

One or more embodiments of the present disclosure may also generally relate to apparatuses, systems, and methods to close an opening, such as a puncture in a vessel wall. Embodiments of the present disclosure include a closure device or closure implant, where a portion of the closure device or closure implant temporarily remains within the patient to close the opening. The portion that remains within the patient subsequently degrades, absorbs, or resorbs into tissues over a period of time.

While the present disclosure will describe a particular implementation of apparatuses and systems, with associated methods, for closing an opening in tissue, it should be understood that any of the systems, apparatuses, and methods described herein may be applicable to other uses, including and not limited to closing existing or formed openings in tissue or body lumens in other locations within a patient's anatomy. Additionally, elements described in relation to any embodiment depicted and/or described herein may be combinable with elements described in relation to any other embodiment depicted and/or described herein.

### Vessel Closure Delivery System

The present disclosure relates to devices, systems, and methods for closing an opening in, for example, a blood vessel wall. For example, the present disclosure includes an anchor, such as an intravascular anchor formed from, in one configuration, a bioabsorbable, bioresorbable, and/or biodegradable material. The anchor may be passed through an opening (e.g., puncture) defined in a wall of a blood vessel and deployed into the vessel lumen. The anchor can then be drawn proximally to draw the anchor into contact with an inner surface of the blood vessel wall. A closure element, such as a cap, can then be deployed on the outside surface of the blood vessel wall to close the puncture.

In at least one example, once deployed within a blood vessel, the anchor (and optionally the cap) may degrade, absorb, or resorb within a predetermined amount of time. For example, between about 36-72 hours, in less than 48 hours, less than about 36 hours, in a day, less than an hour, some other amount of time as desired, or an amount of time within a range defined by any two of the foregoing values. The rate of degradation, absorption, and/or resorption can be selected to correspond to a rate of inactivation for an anticoagulating agent. The rapid degradation, absorption, or resorption of one or more components of the device can allow the anchor, for example, to be left in place after the closure device or closure implant has been deployed by obviating the need for removal of the anchor. By leaving the anchor in place until it degrades, absorbs, or resorbs, damage that may occur by drawing the anchor through the now-closed puncture and/or the deployed closure element can be reduced or eliminated. The anchor also allows for closure of an opening without cross-contamination from extravascular and/or other components.

In addition, the degradation, absorption, or resorption time of the anchor may fall within the time frame of the action of an anti-thrombotic medication being used in conjunction with the treatment of a patient. For example, degradation, absorption, or resorption of the anchor may occur by the time any administered anti-thrombotic medication has been substantially metabolized by the patient. Accordingly, the closure device or closure implant of the present disclosure may reduce the risk of formation of intra-arterial clots associated with the closure of the blood vessel puncture site.

The one or more components of the vessel closure device can degrade, absorb, or resorb within a predetermined amount of time. For example, one or more components of the vessel closure device can degrade, absorb, or resorb within one (1) to eight (8) months, within two (2) months, within three (3) months, within 3.5 months, within six (6) months, or within a time period defined by any two of the foregoing values.

While reference has been made to the anchor remaining in the blood vessel and being subsequently degraded, absorbed, or resorbed by the patient's body, it will be understood that in other configurations the anchor may be deployed and subsequently removed once sufficient closure of the puncture has occurred.

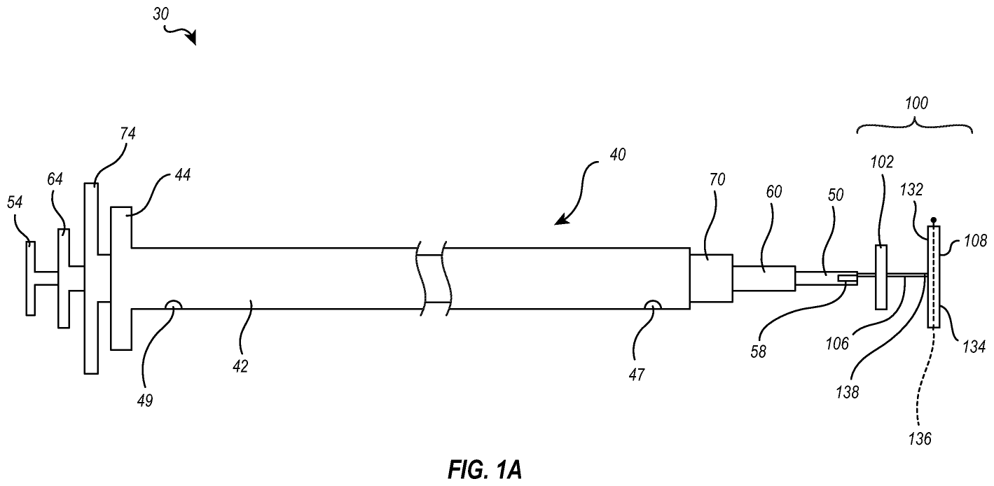

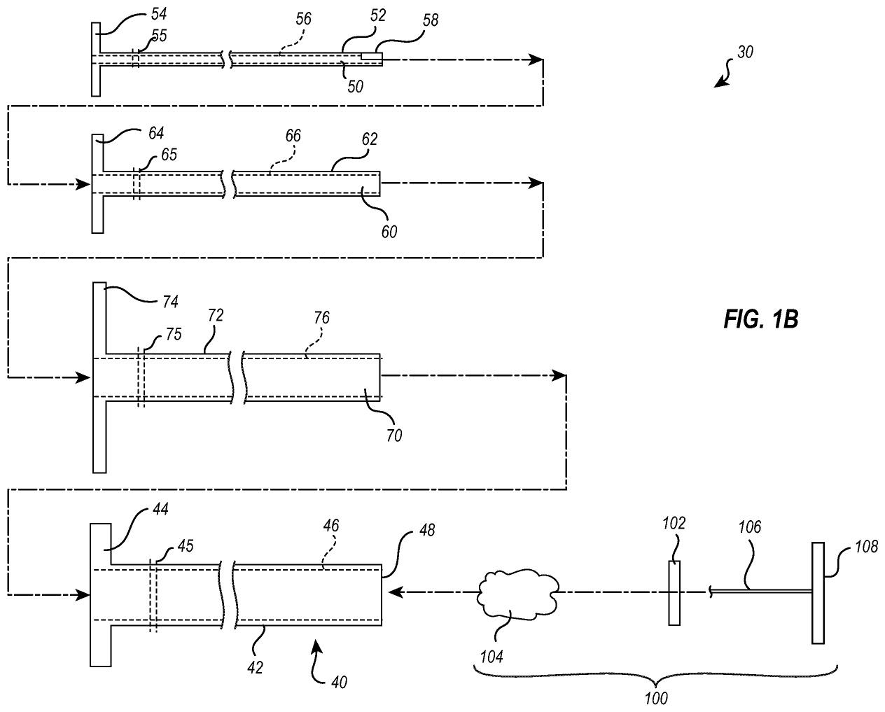

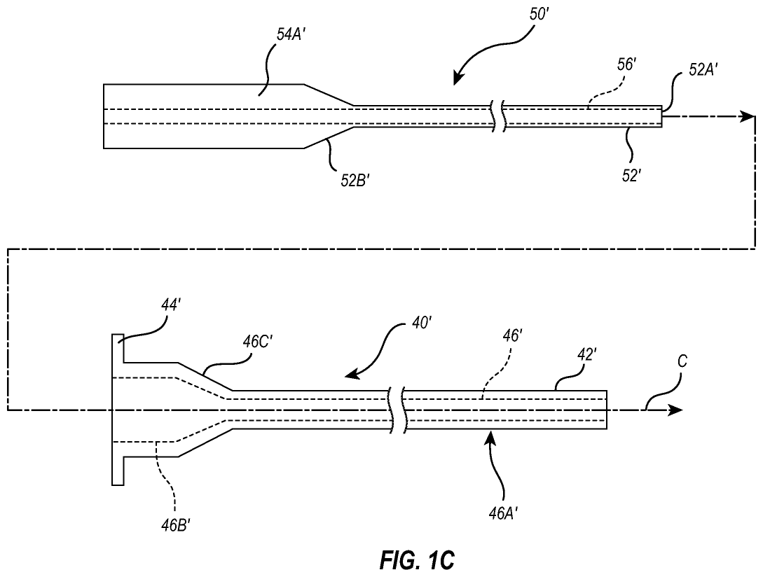

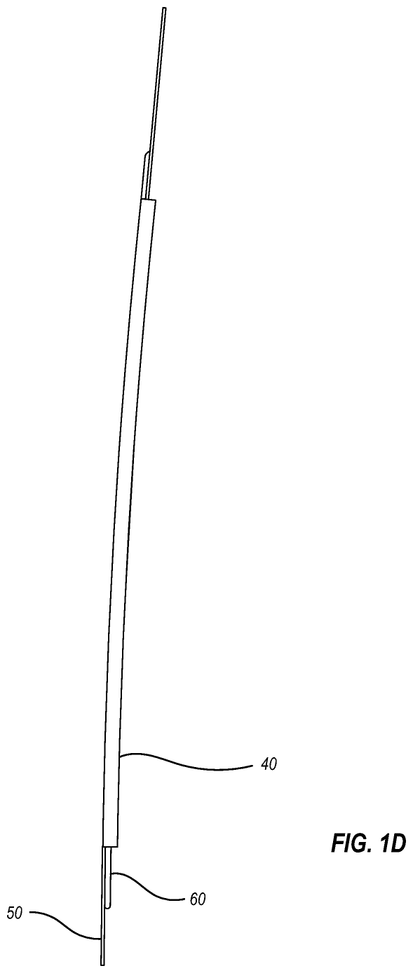

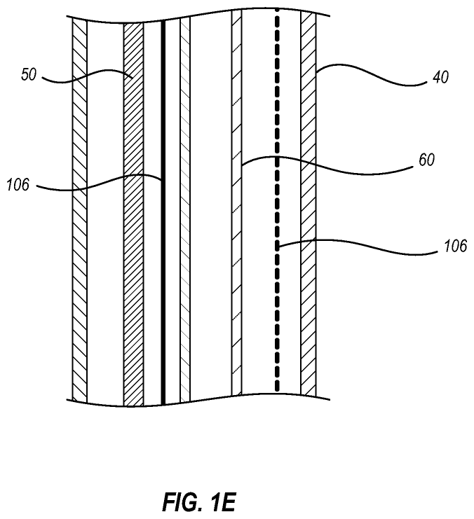

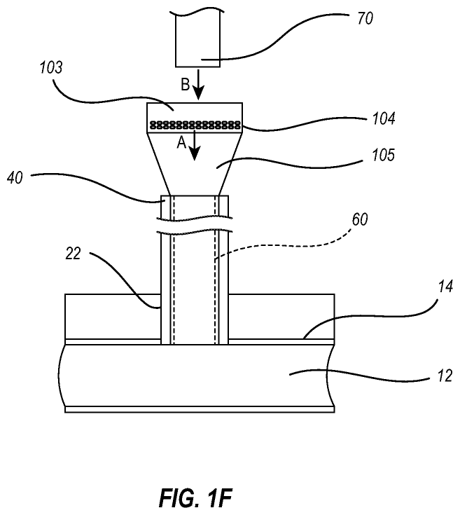

Reference is now made to FIGS. 1A-1B, which illustrate an example of a closure device delivery system or closure implant delivery system 30. As shown in FIGS. 1A-1B, the delivery system 30 can include a delivery sheath 40 with a nested set of actuators 50, 60, 70. The nested set of actuators 50, 60, 70 can be configured to cooperatively deploy a closure device or closure implant 100. The closure device or implant 100 can include an anchor 108, such as an intravascular “foot” or anchor; a closure element, such as a cap 102; a fluid-blocking component 104, such as a sealant (see FIGS. 1B and 2A-4); and a suture element 106. The term fluid-blocking component and sealant will be used interchangeably herein.

In some embodiments, the actuator 50 can be used to deploy the anchor 108, the actuator 60 can be used to deploy the cap 102, and the actuator 70 can be used to deploy the fluid-blocking component 104. In at least one embodiment, the delivery sheath 40 is configured to house the anchor 108, the cap 102, and the fluid-blocking component 104 while the actuators 50, 60, 70 are configured to deploy the anchor 108, the cap 102, and the fluid-blocking component 104, respectively, from the delivery sheath 40. The exemplary delivery sheath 40, actuators 50, 60, 70, anchor 108, and closure device 100 of FIG. 1A will be discussed in more detail with reference to FIG. 1B.

While FIGS. 1A and 1B illustrate the set of actuators 50, 60, 70 as being coaxially disposed within the delivery sheath 40, the actuators 50, 60, 70 can be non-coaxially disposed in the delivery sheath 40. For example, as illustrated in FIGS. 1D and 1E, the actuator 50 can be disposed to a side of the actuator 60. Additionally, while the following discussion provides one manner by which specific actuators can be used to deploy the anchor 108, the cap 102, and the fluid-blocking component 104, it will be understood by those skilled in the art that one of the actuators 50, 60, 70 can deploy any combination of the anchor 108, the cap 102, and the fluid-blocking component 104 in any order or sequence. For instance, while the actuator 60 can deploy the cap 102 and the actuator 70 can deploy the fluid-blocking component 104, in other configurations one of the actuators can be eliminated, such as for example actuator 70, and the actuator 60 can deploy the cap 102, advance the fluid-blocking component 104 toward the cap 102, and deploy the fluid-blocking component 104 through a combination of distal and/or proximal movements in relation to the anchor 108.

In other configurations, the delivery system 30 can include two or more actuators, such as two or more of the actuators 50, 60, or 70, to delivery/deploy the anchor 108, the cap 102, and the fluid-blocking component 104. It is also possible for other combinations of deployment functions to be performed by other individual or combination of actuators.

FIG. 1B illustrates an exploded view of the delivery system 30. As shown in FIG. 1B, the delivery sheath 40 includes an outer housing 42 and a handle or grip portion 44. Each of the actuators 50, 60, 70 include, respectively, a shaft or housing portion 52, 62, 72, a handle or grip portion 54, 64, 74, and distal ends that can cooperate with, respectively, the anchor 108, the cap 102, and the fluid-blocking component 104. For instance, the actuator 50 can include a notch 58 to receive the suture 106 and, optionally, a portion of the anchor 108. An interior lumen 46 is defined in the outer housing 42 that is configured to receive the actuators 50, 60, 70 in such a manner as to allow the actuators 50, 60, 70 to be extended from and retracted within a distal end 48 of the outer housing 42.

Each actuator 50, 60, 70 also includes, respectively, interior lumens 56, 66, 76 to allow for translation of the actuators 50, 60, 70 (either independently or in combinations of 2 or more of the actuators) and the delivery sheath 40. The one or more lumens of the one or more actuators 50, 60, 70 can include one or more valves or seals 55, 65, 75, and the delivery sheath 40 can also include one or more valves or seals 45, to prevent blood flowing from the ends of the delivery sheath 40 and the actuators 50, 60, 70. A translation distance of the actuators 50, 60, 70 can be controlled through contact between adjacent handle or grip portions 44, 54, 64, 74, as appropriate. For instance, the grip portion 44 can limit distal movement of each of the grip portions 54, 64, 74 associated with the actuators 50, 60, 70; grip portion 74 can limit distal movement of each of the grip portions 54, 64; and grip portion 64 can limit movement of the grip portion 54. In this way, over-translation of individual actuators is limited and the anchor 108, cap 102, and fluid-blocking component 104 can be effectively deployed to access and close a tissue opening.

While reference is made to the handle or grip portions limiting actuator translation, it is understood that other approaches can be used for controlling translation. For instance, complementary structures can be formed in the housings and/or the interior lumens to limit translation. In another configuration, the handle or grip portions are combined into a single handle assembly having different actuation controls, such as switches, knobs, sliders, etc. to allow independent or combined movement of one or more of the actuators 50, 60, 70.

In another configuration, as illustrated in FIG. 1C, an interior lumen 46′ of the housing 42′ can include a first portion 46A′ configured to receive a distal end 52A′ and the shaft portion 52′ of the actuator 50′. A second portion 46B′ of the interior lumen 46′ can be configured to receive a proximal shoulder 52B′ of the shaft portion 52′ having the interior lumen 56′. The proximal shoulder 52B′ can be a portion of the handle 54′. More specifically, the second portion 46B′ of the interior lumen 46′ may have a larger width aspect than the width aspect of the first portion 46A′. The width aspects of the first portion 46A′ and the second portion 46B′ can be the diameters thereof or other cross-sectional profiles that are generally transverse to a center axis C of the delivery sheath 40′. For ease of reference, the center axis C of the delivery sheath 40′ will be referenced in describing the position and movement of the other components described herein. In the illustrated example, the interior lumen 46′ may transition from the smaller diameter of the first portion 46A′ to a second larger diameter of the second portion 46B′ at a shoulder 46C′.

Such a configuration can allow the actuator 50′ to translate axially relative to the delivery sheath 40′ within a desired range of motion. In particular, the proximal shoulder 52B′ can translate within the second portion 46B′ of the interior lumen 46′ to advance the shaft portion 52′ within the outer housing 42′ and in relation to the handle or grip portion 44′, to thereby move the distal end 52A′ of the shaft portion 52′ relative to the distal end 42A′ of the outer housing 42′. Interaction between the handle portion 54′ and the shoulder 46C′ can help ensure the distal end 52A′ does not extend beyond a desired position within the outer housing 42′.

In the illustrated example, the first portion 46B′ can also be configured to receive the anchor 108 and the cap 102. Accordingly, as the distal end 52A′ of the shaft portion 52′ is advanced toward the distal end 42A′ and through the first portion 46B′, the distal end 52A′ of the shaft portion 52′ can engage the anchor 108 and/or the cap 102 to move the anchor 108 and/or the cap 102 distally from the outer housing 42′ and out of distal end 42A′. In this way, the anchor 108 and/or the cap 102 are delivered to the puncture site.

Returning to FIG. 1A, the anchor 108 can be configured to move from a pre-deployed state having a pre-deployed width aspect to a deployed state having a deployed width aspect. The anchor 108 is illustrated in the deployed state. The deployed width aspect may be greater than the pre-deployed width aspect. The anchor 108 can have any configuration that allows for this difference in width aspects. In the illustrated example, anchor 108 is configured to rotate or be rotated between the pre-deployed state and the deployed state. In other examples, portions, or all, of the anchor 108 may be configured to unfold from a pre-deployed state having a pre-deployed width aspect to a deployed state having a deployed width aspect that is greater than the pre-deployed width aspect. For example, the anchor 108 may include one or more arms or wings that can be configured to unfold and fold about a plurality of pivot points, hinges, living hinges, bending locations, preferential bending locations, and combinations or modifications thereof.

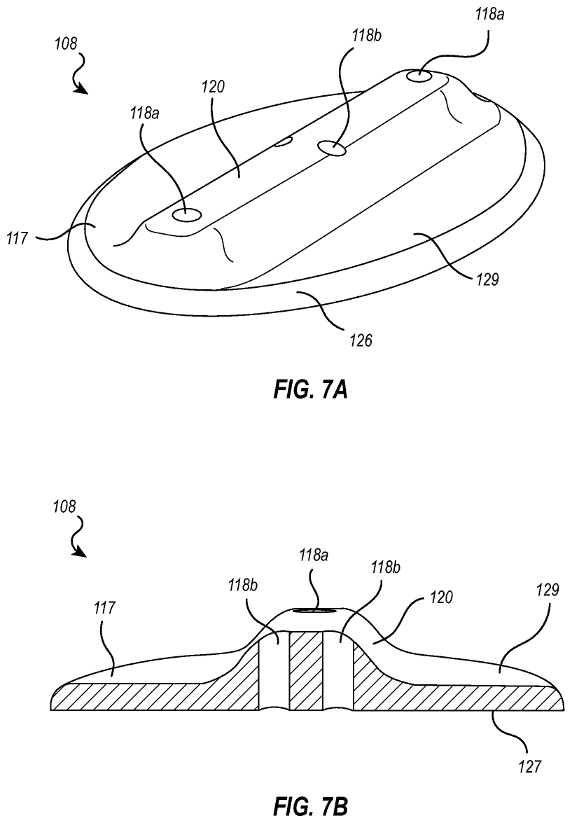

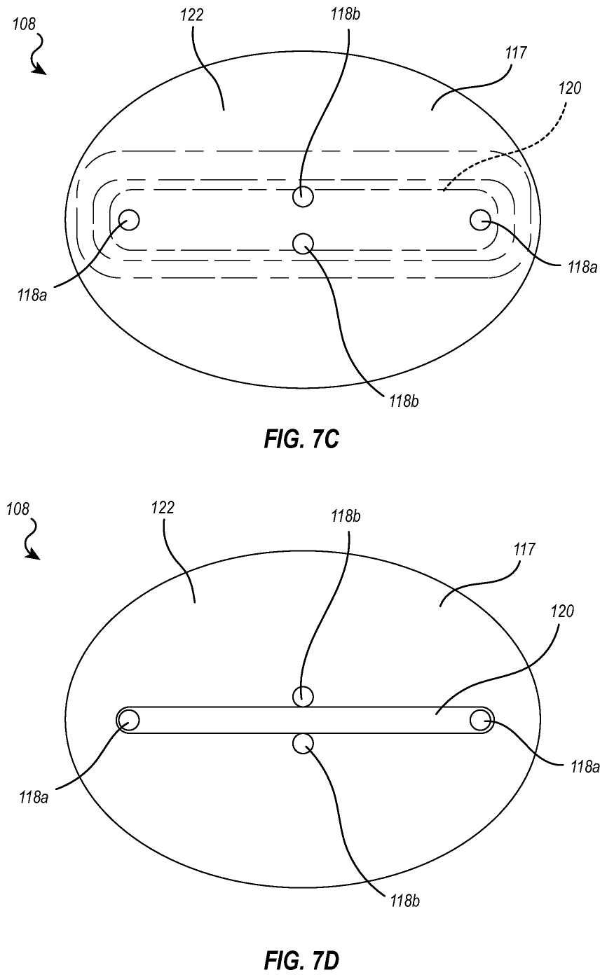

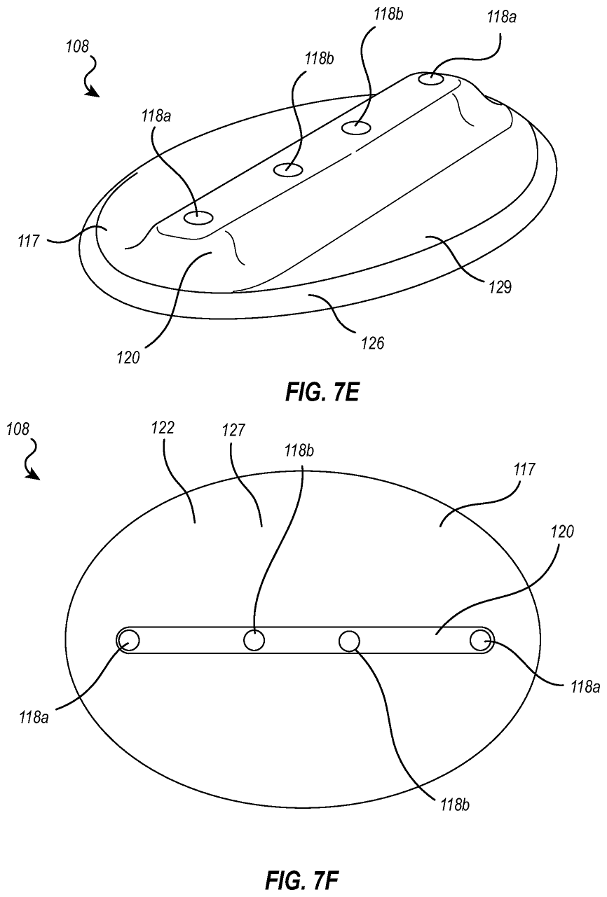

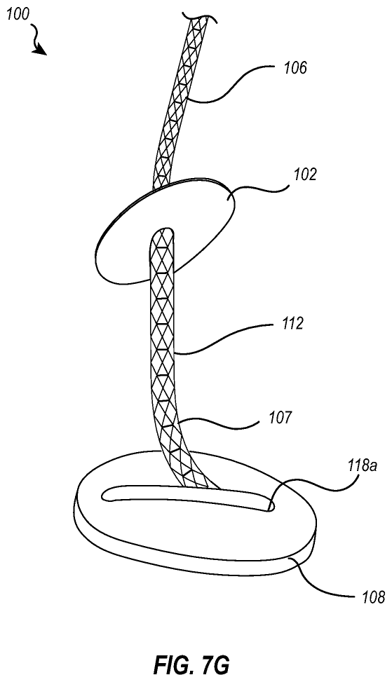

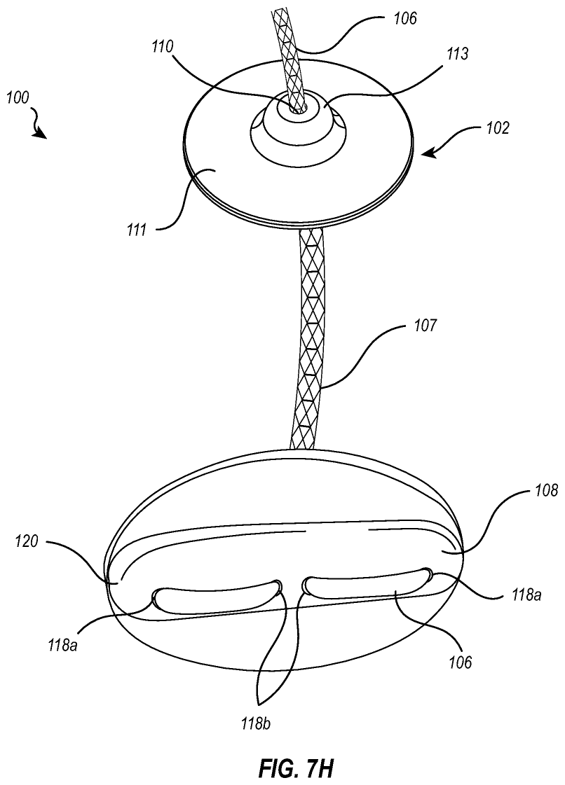

As shown in FIG. 1A, the anchor 108 includes wing members 132, 134 that define a major axis 136 of the anchor 108. The anchor 108 can further include one or more holes or eyelets 138 disposed along a length of the anchor 108. The holes or eyelets 138 can be located at a position that causes the anchor 108 to rotate when a force acting initially parallel to the major axis 136 is exerted on the eyelets 138. Such a configuration can allow the anchor 108 to move from a state in which the major axis 136 is aligned with the central axis C (see FIGS. 1C and 7A-7H) to a state in which the major axis 136 is oriented more obliquely to the central axis C, such as generally perpendicular to the central axis C. As shown in FIG. 1A, the anchor 108 is substantially perpendicular to the central axis C.

This rotation can be accomplished by first applying a distally acting force on the anchor 108 to move the anchor 108 out of the outer housing 42. Then a proximally directed force can be applied to the anchor 108 by way of an interaction between the suture 106 and the eyelets 138. In at least one example, the distally acting force applied to the anchor 108 can be provided from the actuator 50 while the proximally directed force can be applied by way of the suture element 106. The anchor 108 can thus be used to position the delivery system 30 for deployment of the closure element 102.

In one embodiment, the closure element 102 may be configured to close an opening (e.g., a puncture) in a blood vessel wall, as well as at least partially obstruct a tissue tract leading from an external surface of the tissue to the opening. The shape of the closure element 102 may be configured to be housed within the interior lumen 46 (and/or one of the other lumens of the actuators 50, 60, 70). For example, the closure element 102 may be conformable to the shape of the interior lumen 46. In one embodiment, the closure element 102 may be generally cylindrical in shape prior to deployment from the delivery sheath 40. That is, portions (e.g., peripheral portions) of the closure element 102 are at least partially wrapped around, or curved towards, a central portion of the closure element 102. These peripheral portions can curve proximally, distally, and/or transversely to a direction of deployment of the closure element 102, where such direction of deployment is toward the previously deployed anchor 108. Once deployed from the delivery sheath 40, at least a portion of the closure element 102 may be at least partially conformable to a shape of the vessel wall to close an opening in a blood vessel and/or the tissue tract leading to the lumen opening.

The suture element 106 can loop through the anchor 108 such that the suture element 106 passes through or near the closure element 102, and extends proximally into or beyond the housing portion 52 of the actuator 50. In at least one embodiment, a free end of the suture element 106 passes through separate portions or channels of the closure element 102. The suture element 106 can be extended from the closure element 102 and into the actuator 50 by way of the interior lumen 56.

Generally, the structures and components of the delivery system 30 can be formed of polymers, metals, alloys, combinations or modifications thereof. For instance, by way illustration only, the delivery sheath and the actuators can be formed from metal hypotubes, polymer tubes, composite tubes having a multilayer configuration, or other tubular structures, optionally including reinforcing members or braids. The delivery sheath and the actuators can range in outside diameter from about 6F to about 10 F, from about 2 mm to about 4 mm, from about 2 mm to about 3.33 mm, or other sizes as known to those skilled in the art.

FIG. 1F schematically illustrates an example of the fluid-blocking component or sealant 104 housed within a chamber 103. The chamber 103 is located or disposed at a proximal end of the delivery system 30. The sealant 104 is stored within the chamber 103 and is configured to be moved distally through the delivery system 30 via the actuator 70. For example, the actuator 70 engages with the sealant 104 and advances the sealant 104 distally through and out of the chamber 103, and through and out of a funnel 105. Once the sealant 104 is advanced through the funnel 105, the sealant 104 is advanced along and through the actuator 60, which is contained within the delivery sheath 40. Upon distally exiting the actuator 60 and, thus, the delivery sheath 40, the sealant 104 can be exposed to blood and/or other bodily fluids for activation, as described herein.

### Vessel Closure Device

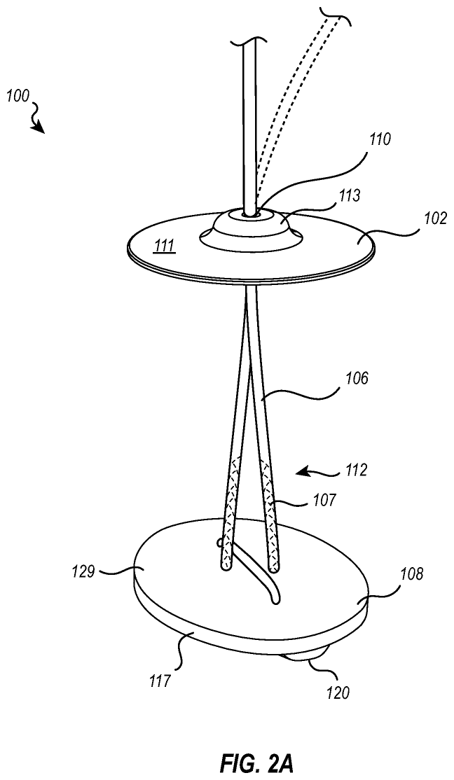

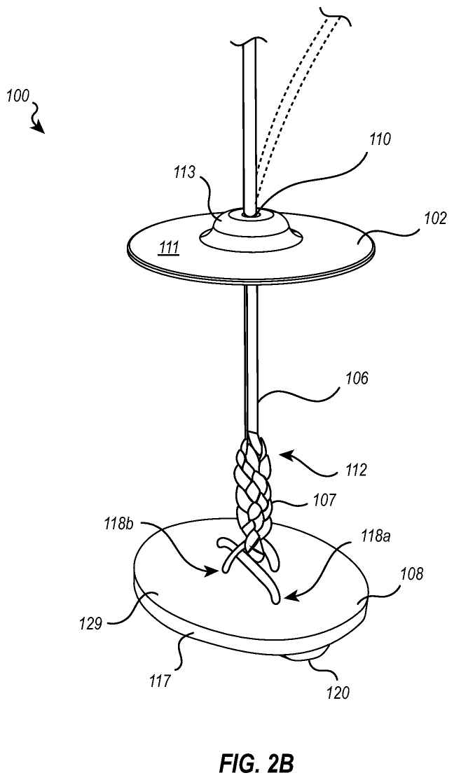

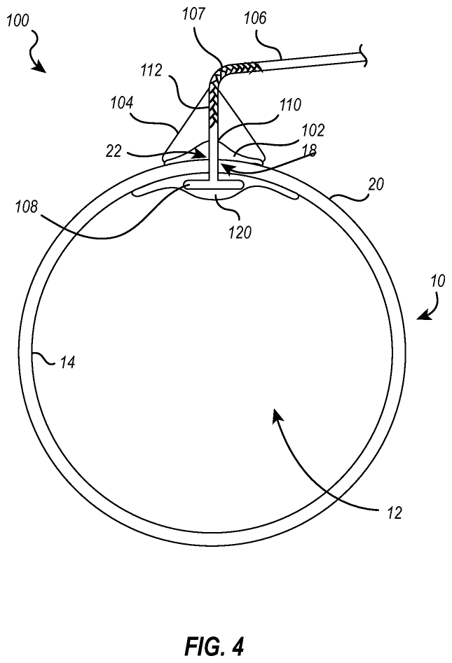

FIGS. 2A-2B illustrate an example of the closure device or implant 100. In some embodiments, the closure device 100 can be a fully bioabsorbable vessel closure implant including intravascular and extravascular components. The extravascular components can include a closure element or cap 102 (hereinafter “extravascular cap” or “cap”) and a fluid-blocking component 104, such as a bioabsorbable sealant (see FIGS. 4-6B). The intravascular components can include an intravascular foot or anchor 108 and a suture 106, both of which can be bioabsorbable. As mentioned above, in other configurations, the anchor 108 can be temporarily deployed, with the extravascular components being fully bioabsorbable (such as through degradation, absorption, and/or resorption).

As illustrated, the cap 102 includes a central lumen 110, a raised portion 113 and a cap surface 111. In some embodiments, the lumen 110 is sized to accommodate a diameter of the suture 106. In some embodiments, the lumen 110 is sized to accommodate a diameter of two free ends of the suture 106 (such as illustrated in FIG. 2B). The suture 106 can be threaded distally through the lumen 110, through the anchor 108, and then proximally threaded back through the lumen 110 (indicated by the dotted line portion of the suture 106). Thus, both free ends of the suture 106 will extend proximally from the cap 102.

The anchor 108 includes a keel 120 (discussed more fully below) and a surface 129. As discussed elsewhere, the suture 106 can be threaded through holes or eyelets within the anchor 108 to thereby secure the suture 106 to the anchor 108. Such a configuration also allows for any forces applied to the suture 106 (i.e., pulling or tensioning the suture 106) to be transferred to the anchor 108. For example, when a physician or practitioner exerts a proximal pulling or tugging force on the suture 106, a proximal pulling or tugging force will be exerted on the anchor 108, moving the anchor 108 in a proximal direction.

As shown in FIG. 2B, the suture 106 may be braided or otherwise entwined with itself prior to looping back through the lumen 110 of the cap 102. This results in a thicker suture portion 112. Additionally, and/or alternatively, the thick suture portion 112 may be formed by or engaged with an elongate member 107. The thick suture portion 112 can then form an interference fit with the lumen 110. Such a fit prevents the cap 102 from undesirably advancing distally beyond a desired position. In some embodiments, two sutures 106 may be threaded through the cap 102 (via lumen 110) and pass through or cooperate with the elongate member 107. The two sutures 106 may be braided to and/or with the elongate member 107 to create the thick suture portion 112.

In each case, (i.e., two adjacent non-braided suture rails, two adjacent braided suture rails, braided suture and elongate member, or a suture end braided into another portion of the suture after being interwoven through 2 or more holes of the anchor 108) the formed thick suture portion 112 can form an interference fit with the lumen 110, which is narrow relative to the thick suture portion 112, to secure the cap 102 in the desired position. The thick suture portion 112 can have a diameter ranging from about 0.020″ (0.508 mm) to about 0.040″ (1.016 mm), from about 0.024″ (0.6096 mm) to about 0.034″ (0.8636 mm), from about 0.028″ (0.7112 mm) to about 0.030″ (0.762 mm).

The suture 106 can be made of a bioabsorbable material. For example, the suture 106 can be a multifilament or braided absorbable suture, such as those available from VITREX®. In one configuration, the suture is a braided 3-0 suture. It may be advantageous for the suture to have a high tensile strength which can maintain its integrity under the application of about 3 pounds-of-force (lbf.) to about 6 lbf., although other sutures can accommodate application of forces ranging from about 1 lbf, to about 16 lbf., from about 1 lbf. to about 8 lbf., from about 2 lbf. to about 6 lbf., from about 2.5 lbf. to about 5 lbf., or about 2 lbf. Other ranges between any two such values are also possible.

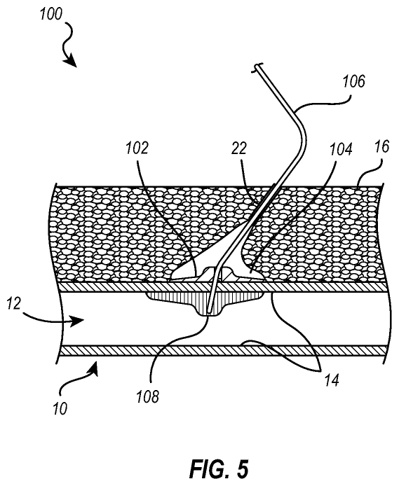

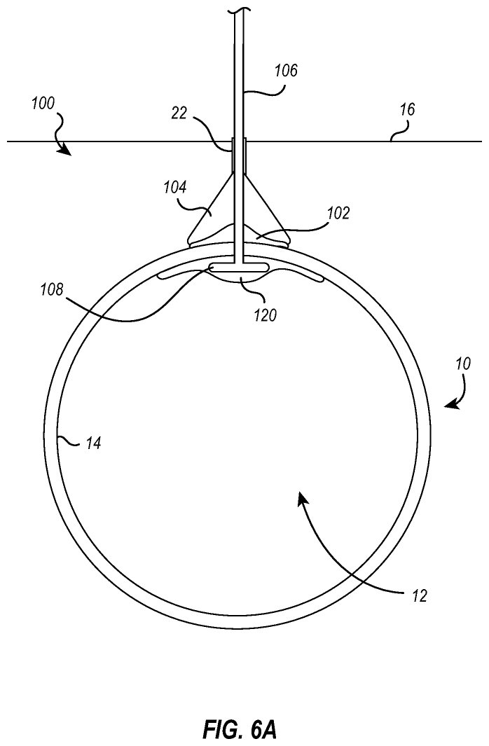

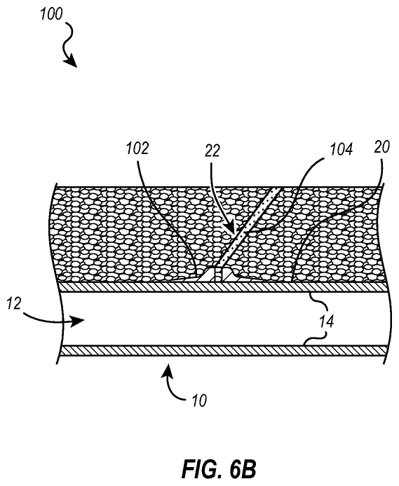

The extravascular cap 102 can be made from bioabsorbable materials and be of sufficient size and geometry to prevent it from passing through the puncture access site 18 at the surface of the blood vessel 10 (see FIGS. 5 and 6A). The size and geometry of the cap 102 can significantly increase patient safety by preventing extravascular components from passing through the access site 18 during and/or after deployment. The cap 102 can have a diameter ranging from about 1 mm to about 10 mm, from about 3 mm to about 8 mm, from about 4 mm to about 5 mm, or a range defined by any two of the foregoing values. The cap 102 can have another size and shape based upon the specific dimensions of the access site 18, so that the cap 102 does not pass through the puncture/access site 18 and into the vessel lumen.

The cap 102 can have a low-profile and be made from a biodegradable material. The cap 102 can also have a desired flexibility to conform to the anatomy at the access site 18 (especially in vessels with significant calcification present) and provide more effective sealing than would rigid materials. The cap 102 can be deployed through an access tissue tract 22 and placed on top of the vessel 10 (see FIGS. 4-6B), acting as the primary extravascular seal.

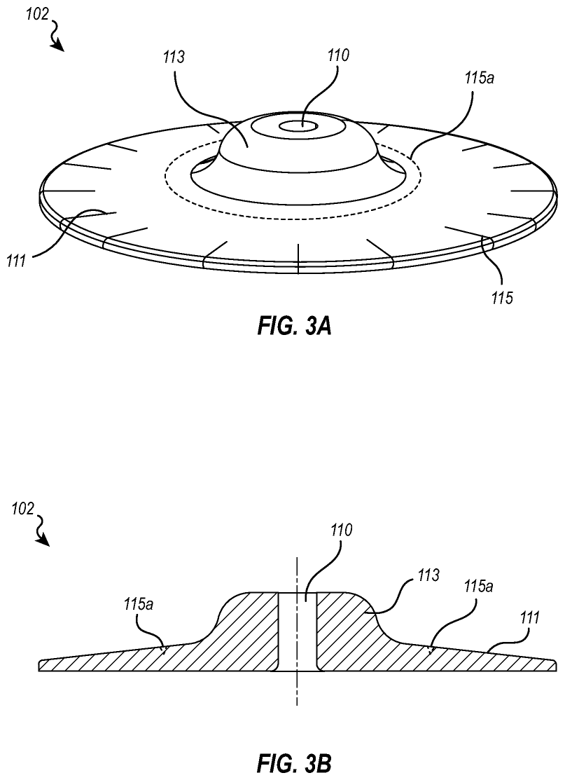

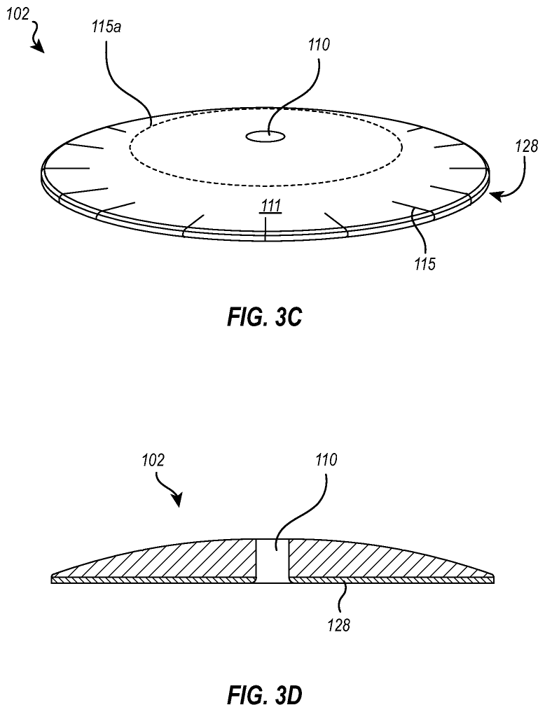

Turning to FIGS. 3A-3B, illustrated is one configuration of the cap 102. As illustrated, the cap 102 can have a generally circular disk shape with a consistent outer perimeter or circumference. In other embodiments, the shape of the cap 102 can be interrupted (e.g., star-shape) which can impart the cap 102 with increased flexibility allowing it to conform to the access tract 22, which is typically narrow. The cap 102 can include a medial portion 113 which may be raised relative to a surrounding surface 111 of the cap 102. Alternatively, as illustrated in FIGS. 3C-3D, the medial portion 113 may provide a domed shape to the cap 102, but otherwise be continuous and smooth with the surrounding surface 111.

The medial portion 113 can have a thickness of about 0.050 mm to about 5 mm, from about 0.10 mm to about 2 mm, from about 0.10 mm to about 0.5 mm, or various other thicknesses. The cap surface 111 can include relief cuts 115 which may facilitate increased cap flexibility and conformance to the access tract 22 leading to the vessel 10.

The relief cuts 115 can radially extend about a longitudinal axis of the cap 102, and can be inclined, curved, or non-linear along the surrounding surface 111, or combinations and/or modifications thereof. Alternatively, or in addition to the relief cuts 115, relief cuts 115a can have a generally circular form disposed around the medial portion 113, such as to circumscribe, surround, and/or encircle all or a portion of the medial portion 113, penetrating only partially through the thickness of cap 102. The relief cuts 115a can modify the flexibility of the surface 111 to improve conformance of the cap 102 to the access tract 22 and resist entry of the cap 102 into the vessel. The cap 102 can have a mass ranging from about 4.0 mg to about 10.0 mg (for 4 mm to about 6 mm diameter cap). With a lower overall mass, less force is required to hold the cap 102 in place via, for example, frictional engagement between the cap 102 and the suture 106. This results in a smaller and lighter overall system, thereby making positioning of the closure device 100 within the patient simpler and with reduced overall impact on the patient's recovery.

The access tract 22 (see FIGS. 4-6B) is typically size-restricted, circular, and formed at an angle in relation to the vessel wall. The cap 102 can be configured to slide down and through a delivery system 30, through the access tract 22, and be deposited on top of a vessel 10. The suture 106 can then be pulled to tension the cap 102 and intravascular anchor 108 towards each other, thereby sealing the access site 18. As shown in FIGS. 3A-3D, the cap 102 can include a lumen 110 in the medial portion 113 through which the suture 106 can be threaded to attach the suture 106 to the anchor 108. The lumen 110 can have a diameter ranging from about 0.010″ (0.254 mm) to about 0.020″ (0.508 mm), from about 0.012″ (0.3048 mm) to about 0.017″ (0.4318 mm), or from about 0.014″ (0.3556 mm) to about 0.015″ (0.381 mm).

As discussed, the lumen 110 can be sized to accommodate the suture 106 which has a certain diameter. For instance, as illustrated FIGS. 2A-2B, with the suture 106 looped around the anchor 108, two rails or portions of the suture 106 can pass through the lumen 110 and proximally along the delivery device 30. Optionally, portions of two sutures 106 can be braided together with two suture rails extending proximally from the cap 102. Alternatively, as illustrated in FIG. 2B, the suture 106 is looped back on itself and braided into itself to increase size (e.g., width) of a portion of the suture 106 that interference fits or otherwise engages with the lumen 110, with a single rail extending proximally along the delivery device 30. In still another case, two sutures 106 can pass through or cooperate with an elongate member 107 (such as another suture portion or braided tubular member), shown in phantom in FIGS. 2A-2B, and be braided to and with the elongate member 107, to increase a size of the portion(s) of the suture 106 disposed within the cap 102 and/or lumen 110. One or more elongated member(s) 107 can optionally be inserted into the one or more sutures 106 to increase their dimensions.

The cap 102 can be initially positioned on a proximal end of the suture 106. That is, the end of the suture 106 which does not have a diameter larger than the diameter of the lumen 110 of the cap 102. In some embodiments, the suture 106 has a diameter that changes along a longitudinal axis of the suture 106. When the cap 102 is advanced distally along the suture 106 toward the external vessel surface 20 at the access site 18, the thick suture portion 112 causes an interference fit that can lock the cap 102 in place, resulting in an immediate (or substantially immediate) dry close.

The interference fit eliminates the need for the use of a knot to maintain the dry close formed when closure device 100 is implanted and tightened. Use of a knot can pose serious risk to a patient if the set tension on the suture becomes overtightened. The suture can become stressed by a patient walking or coughing causing the suture to over tension and break. The disclosed interference fit is advantageous because it is knotless and the flexibility of the cap can adapt to force applied to the suture.

In addition to, or instead of the interference fit between the cap 102 and the thick suture portion 112, the cap 102 can optionally include an adhesive applied to a side of the cap contacting the vessel surface 20. For example, as illustrated in FIGS. 3C-3D, the cap 102 can include an adhesive layer 128 that bonds to the extravascular tissue when the cap 102 is advanced towards the anchor 108. The adhesive for the adhesive layer 128 can be a non-migrating adhesive, in that it will not flow through the puncture or access site 18 as the extravascular tissue is sandwiched between the cap 102 and the anchor 108. Such adhesive can include a non-expanding glue, such as a non-expanding polyethylene glycol (PEG); a glue protein, such as a barnacle glue; cross-linked gelatins; (non-biologic) cyanoacrylates; polyurethane adhesives; other glues or adhesives; and/or combinations and modifications thereof. More generally, the adhesives can use cross-linking mechanisms that rely on chemical conjugation between reactive groups, free radical polymerization, oxidation reduction reaction, and/or biological or biochemical coupling.

FIGS. 4-6B illustrate an example of a deployed closure device 100 including a second extravascular component as a fluid-blocking component or sealant 104. The sealant 104 can be an active biologic material, such as polyethylene glycol (PEG), fibrin sealants, copolymer of glucosamine and N-acetyl glucosamine, dextran (a complex branched glucan (a polysaccharide derived from condensation of glucose), polypeptide adhesive structures, adhesive protein containing L-3,4-dihydroxyphenylalanine (L-DOPA), adhesive protein containing DOPA and phosphoserine, collagen, polyacrylic acid, cross-linked with allyl sucrose or allyl pentaerythritol, polyacrylic acid, cross-linked with divinyl glycol, Acrylic resinous polymer composed of methyl-2-cynoacrylate units, or another fully bioabsorbable sealant-type material that could be optionally incorporated into a shaped, flexible substrate. The sealant material is activated by fluids present in the patient's tissue tract, such as blood or other bodily fluids, and can be protectively stored inside any one of the sheath/actuators or a chamber of the delivery device until positioned directly on top of the cap 102.

Once advanced into the desired location, the fluid-blocking component or sealant 104 can be exposed to blood or other bodily fluids. For example, unsheathing the sealant 104 and positioning the sealant 104 into direct contact with the tissue allows for a reaction of the sealant 104 with blood and/or other present fluids. This reaction can cause the sealant 104 to expand and absorb blood and/or other fluids, and bond to surrounding tissue(s) and the cap 102. The sealant 104 can act as a glue and aid with “locking” the cap 102 in place on the blood vessel 10. The sealant 104 also actively coagulates the entire access tract 22 due to the reaction with blood and/or other bodily fluids. The chemical formulation, quantity, carrier matrix, and/or dimensions of the sealant 104 can be selected specifically to provide one or more of the functions of locking the sealant 104 in place (e.g., against cap 102), locking the cap 102 in place, to provide a fast acting and leak-free dry close, and reduce tissue tract oozing.

For instance, the sealant 104 can form a plug having a length of about 1 mm to about 10 mm, such as 4, 5, 6, 8 mm, or a length defined by any two of the foregoing values. The sealant 104 can optionally be trimmed to length in the patient along with the suture 106 after deployment. Alternatively, the sealant 104 can extend the full length of the tissue tract 22 and be trimmed to fit the patient. When the sealant 104 is formed of a matrix, the matrix can have an area of about 0.012 square inches to about 0.12 square inches, about 0.12 square inches to 0.6 square inches, or about 0.6 to 1.0 square inches. The matrix material can be thin and flexible such that it can be wrapped around the suture 106 in the delivery system to fit inside a tube for delivery to the implant location. This results in a volume of the fluid-blocking component or sealant 104 (optionally including a matrix containing a sealant such as PEG or other biocompatible material) of between about 0.004 to about 0.040 cubic inches in volume, about 0.040 to about 0.100 cubic inches, or about 0.100 to about 0.400 cubic inches in volume.

The sealant 104 can be deployed so that it is disposed on/over the suture 106. The sealant 104, therefore, can be deployed in a flowable composition without a carrier matrix or can be formed as part of, or with, a carrier matrix. For instance, the sealant 104 can be disposed around the suture 106 in a generally cylindrical configuration, can be bonded to the suture 106 itself, can be bonded to the cap 102, and/or combinations or modifications thereof. Because the sealant 104 is positioned/deployed proximal relative to the cap 102, the sealant 104 can actively coagulate around, over, and through the access tract 22 and, optionally, actively coagulate all of access tract 22 up to the surface of the skin 16.

Sealant 104, as shown in FIGS. 4-6B, can have a conical configuration when deployed. In other embodiments, the sealant 104 can have a continuous or uniform thickness along its length. The cap 102 can displace tissue at the access site 18 because the cap 102 can be larger than the opening/access site 18. The sealant 104 can also fill the space created by the displaced tissue. The sealant 104 can be formed of material(s) having properties which can cause it to swell, from its original size, upon contacting blood and/or other bodily fluids, causing it to effectively cover and reinforce the seal formed by the cap 102. The sealant 104 can swell from its original size to about 1 to 6 times the original size, from about 2 to 4 times, or from about 2.5 to 3.5 times, or a range defined by any two of the foregoing values. It can be advantageous to optionally have the sealant 104 expand up (i.e., expand proximally) the access tract 22 as close as possible to the skin 16 to mitigate or reduce any bleeding. Such expansion may occur very quickly (e.g., nearly instantaneously) after contact with the expanding bodily fluids, e.g., from about 15 seconds to about 5 minutes, from about 30 seconds to about 4 minutes, from about 1 minute to about 3 minutes, from about 15 seconds to about 1 minute, from about 15 seconds to about 45 seconds.

When the sealant 104 has a predetermined conical or tapered shape, the sealant 104 can be formed as a separate sealant component with a hole through a center, or other locations, to allow the sealant 104 to be threaded onto the suture 106. More generally, the suture 106 may be threaded through one or more points through and/or around the sealant 104. The sealant component could be a foam matrix or other formed substrate that a biocompatible material can be infused into and then formed into the desired shape, such as PEG. The sealant 104 can be a combination of two or more components which can be loaded into one of the actuators 50, 60, 70. The two or more components would then be simultaneously activated by pressing down the handle or grip portions of the actuators 50, 60, 70 to expose the sealant 104 to blood and/or other bodily fluids. The two or more components can include one or more flowable component, with or without a matrix having a preformed shape or being biased to a particular shape.

In other embodiments, the sealant 104 and the cap 102 can be deployed together as if they are one component. The cap 102 can cover the access site 18 and the sealant 104 can be activated on top of and above the cap 102 to seal the access tract 22. In other embodiments, as illustrated schematically in FIG. 1F (in which the actuator 50, the anchor 108, and the cap 102 are omitted for case of explanation), the sealant 104 can be stored in a chamber 103 at a proximal end of the delivery system 30. The sealant 104 can be stored in a generally planar or flat-sheet form, and optionally biased to that generally planar or flat-sheet form. The sealant 104 is advanced to the cap 102 through a funnel 105, or other proximal deployment port, that curls, folds, or otherwise changes the planar or flat-sheet form to a form capable of being advanced toward the cap 102.

In one configuration, the actuator 70 can be used to advance the or sealant 104 from the chamber (arrow A) along the actuator 60 to deploy the sealant 104 from within the actuator 60. For example, the actuator 70 is distally advanced (arrow B) when the actuator 60 is partially withdrawn proximally from engagement with the cap 102. Such partial proximal withdrawal typically occurs following sandwiching of the tissue between the cap 102 and the anchor 108, through movement of another actuator or structure, or combinations or modifications thereof. The movement exposes the sealant 104 to blood and/or other fluids causing the sealant 104 to react and expand.

Generally, when introducing a coagulant or sealant, there is a risk of introducing embolizing material into the vessel 10 which can cause a clot and thereby threaten a limb. Emergency surgery may be required to remove the material. This risk can be mitigated by the configuration of closure device 100 due to the use of the cap 102 to first cover the access site 18 so that the extravascular sealant 104 cannot pass into the vessel 10.

The low-profile cap component 102, including a stable degradable, absorbable, or resorbable material (i.e., material that does not expand or aggressively bond to tissue), plus the active sealant 104 material on top, combined as an extravascular implant, is unique and distinguishes this design from other closure devices. Specifically, the disclosed design prevents migration of extravascular or other undesired components into the punctured vessel, thereby sealing the vessel and preventing infection.

Turning now to FIGS. 7A-7H, the closure device 100 can include an intravascular anchor 108, for example, a graft-type anchor. FIGS. 7C-7D illustrate one embodiment of the anchor 108, with FIG. 7C illustrating a lumen-facing side 117 of the anchor 108 and FIG. 7D illustrating an intima-facing side 127 of the anchor 108. Similarly, FIGS. 7E-7F illustrate a lumen-facing side 117 of the anchor 108 and an intima-facing side 127 of the anchor 108, respectively. FIGS. 7G-7H illustrate a substantially assembled, but not implanted, closure device 100.

The anchor 108 can include one or more of the following elements: 1) a large surface area in an elongate shape otherwise referred to as elongate member 117; 2) a central keel 120 which can provide suture attachment sites and overall rigidity; 3) a flexible portion or membrane 122 which can conform to a vessel wall; 4) holes, eyelets, or other structures 118 which can provide for suture attachment to the anchor 108 and an extravascular component (e.g., cap 102) of a closure device 100; and 5) flexible edges 126 of the flexible portion or membrane 122 which can allow for storage in a cylindrical state to permit delivery of the closure device 100 to the vessel 10. The anchor 108 can be formed of multiple sub-components that are joined together. Alternatively, the anchor 108 can be formed as a monolithic component where one or more of the elements are formed as a single component, such as through casting or through machining of a starting workpiece.

The anchor 108 can be formed of a bioabsorbable material, while having flexibility properties that allow the anchor 108 to be curled up into a smaller profile inside of a delivery sheath, such as the delivery sheath 40. This allows a larger sealing surface that can unfurl once free of the delivery sheath 40. The anchor 108 is attached to the suture 106 using a pattern of holes or eyelets 118 that can distribute tensile load more widely across the breadth of the anchor 108, which prevents fracture from a high concentration of force during device deployment.

The anchor 108 can have a curved profile in order to better conform to the curvature of the vessel wall. The anchor 108 can also have an enlarged central portion or a keel 120. The keel 120 can help to reinforce the seal formed over the access site 18 by the closure device 100, as well as provide one or more suture attachment points. The rigidity of the keel 120 can provide mechanical leverage and a robust location to advance and eject the anchor 108 out of the delivery sheath 40. The keel 120 can have a thickness of about 0.5 mm to about 0.8 mm, of about 0.6 mm to about 0.9 mm, of about 0.7 mm to about 1.0 mm, or other thickness to provide the desired suture attachment location. Surrounding the keel 120 is the elongate member 117. The materials forming the elongate member 117 can be the same as the keel 120, such as a bioabsorbable material, with the material a have a durometer ranging from about 50 Shore A to about 100 Shore A, from about 80 Shore A to about 90 Shore A, durometers as chosen based upon the closure location, and/or a durometer within a range defined by any two of the foregoing values. The elongate member 117 can have thinner, flexible sections relative to the keel 120, where the thinner sections can conform to the curved vessel wall 14. The flexibility of the thinner sections can also allow the anchor 108 to conform to any unique calcification buildup in the vessel 10. Calcification typically follows a puncture or wound. The elongate member 117 can have an ellipse or oval shape with a minor axis dimension from about 2.0 mm to about 10.0 mm, from about 3.0 mm to about 5.0 mm, or about 4 mm, while a major axis can range from about 4.0 mm to about 12.0 mm, from about 6.0 mm to about 8.0 mm, or about 6.0 mm. It is understood that the configuration of the anchor 108, and more generally, the closure device or implant 100 can be varied based upon the particular opening in a blood vessel to close, so that the dimensions can be adjusted to accommodate, generally, 5F-8F openings or openings larger than 8F or smaller than 5F.

The ridge or keel 120 can run the length of the central axis of the elongate member 117 and can impart rigidity where the suture 106 can be attached. The suture 106 can be attached through suture attachment points or holes 118 in the keel 120. One or more holes 118 can provide points through which the suture 106 can be threaded to attach the anchor 108 to the cap 102 and the sealant 104. The holes 118 can be evenly or non-evenly spaced along the length of the keel 120. The spacing of the holes 118 can help spread applied tensile loads across a desired length of the anchor 108, such as all or some portion of the length of the anchor 108. The spacing can also prevent fracture, ripping, or tearing of the anchor 108 under a load. In the embodiment shown in FIGS. 7A-7H, free distal ends of the suture 106 can first be threaded through each of the outermost holes 118a, then both can be threaded through the middle holes 118b, and then up through the access/puncture site 18. The free distal ends can also be braided back onto the suture 106 to form the thick suture portion 112 (see FIG. 2B).

The anchor 108 can be injection molded, cast, stamped, machined, and combinations or modifications thereof. The anchor 108 can include one or more bioabsorbable materials, bioabsorbable polymers, or bioabsorbable elastomers depending on the degree of strength, stiffness and absorption rate desired. The anchor 108 structure can be formed of a homogenous material mixture where flexibility is adjusted through a combination of geometry and material formulation. A secondary adhesive material may be attached or bonded to the intima-facing surface 127 of the anchor 108 to increase attachment strength and improve sealing performance against the interior of the blood vessel wall. The anchor 108 provides a safe manner for the sealant 104 to interact directly with the blood vessel tissue without risk of embolizing into the blood vessel lumen because the sealant 104 is attached to the anchor 108. Exemplary bioabsorbable materials from which any of the described components may be formed can include, for example, and not by way of limitation, Polyglycolic acid (PGA), Polylactide (PLA), Poly-L-Latic acid (PLLA), Polycaprolactone (PCL), Poly-DL-lactic acid (PDLLA), Poly trimethylene carbonate (PTMC), Poly para-dioxanone (PPDO), combinations and/or modifications thereof. An exemplary bioabsorbable triblock copolymer that may be used for any such components is described in U.S. Provisional Patent Application Ser. No. 63/505,920 filed May 31, 2023, and entitled “ABA TRI-BLOCK COPOLYMER AND BIORESORBABLE IMPLANTS MADE THEREWITH”, which is herein incorporated by reference in its entirety. More generally, the materials forming the anchor 108 or other components can have a durometer ranging from about 50 Shore A to about 100 Shore A, or from about 80 Shore A to about 90 Shore A. Where the anchor 108 is temporarily deployed, the anchor can be formed of a non-bioabsorbable material, such as polyvinyl chloride (PVC), Polyether ether ketone (PEEK), Polytetrafluorethylene (PTFE), nylon, silicone, urethane, thermoplastic elastomers like Polyether block amide (PEBAX), polyethylene terephthalate (PET), Fluoropolymers, or other biocompatible materials, combinations and/or modifications thereof.

The anchor 108 can have a mass ranging from about 4 mg to 8 mg (for a 4 mm×6 mm ellipse), from about 8 mg to about 16 mg (for a 5 mm×7 mm ellipse), or from about 15 mg to 30 mg (for an 8×10 mm ellipse). With a lower overall mass, less force is required to hold the anchor 108 in place via frictional engagement between the cap 102 and the suture 106. This results in a smaller overall system, thereby making positioning of the anchor 108 within the patient simpler and with reduced overall impact on the patient's recovery.

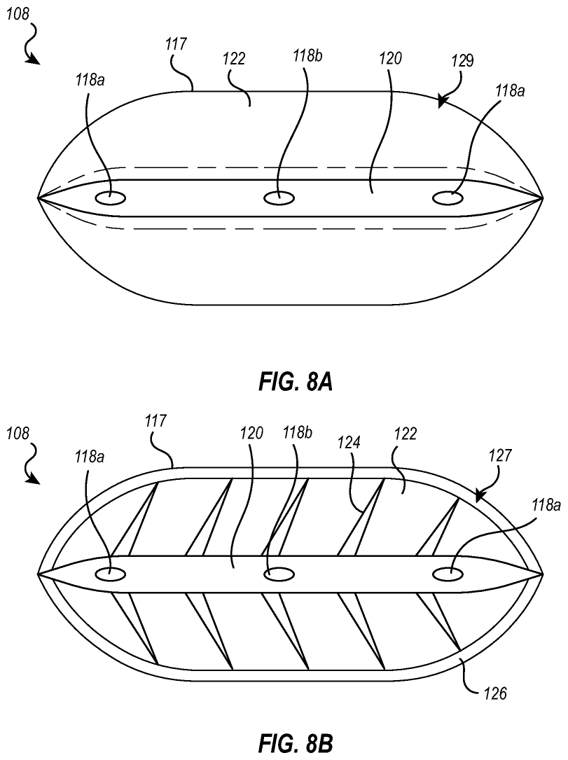

FIGS. 8A-8B illustrate another example embodiment of the intravascular anchor 108. In FIGS. 8A-8B, the anchor 108 includes a lumen facing side 129 (FIG. 8A) and an intima facing side 127 (FIG. 8B). As with the anchor 108 of FIGS. 7A-7H, the anchor 108 can include an elongate body 117 having a flexible member or membrane 122, and a keel 120 positioned along a central axis of the elongate body 117 and spanning the length of the elongate body 117. The keel 120 can provide adequate stiffness for attachment of the intravascular anchor 108 to the extravascular element (e.g., cap 102) of the closure device 100 by the suture 106.

The keel 120 can be raised relative to the lumen facing side surface 129 of the anchor 108, which can help to maintain the position of the anchor 108 on the vessel wall 114. The intima side 127 of the anchor 108 can include a plurality of ribs 124 radiating outward from the keel 120 to a raised edge 126 forming the perimeter of the elongate body 117. The ribs 124 and raised edge 126 provide for encapsulation of localized plaque on the vessel wall 114. The stiffness of the raised edge 126 of the anchor 108 may be correlated to the stiffness and/or pattern, number, and/or thickness of the ribs 124 radiating from the keel 120. The width and taper of the ribs 124 may be varied to influence the compliance or the stiffness of the edge 126 of the anchor 108.

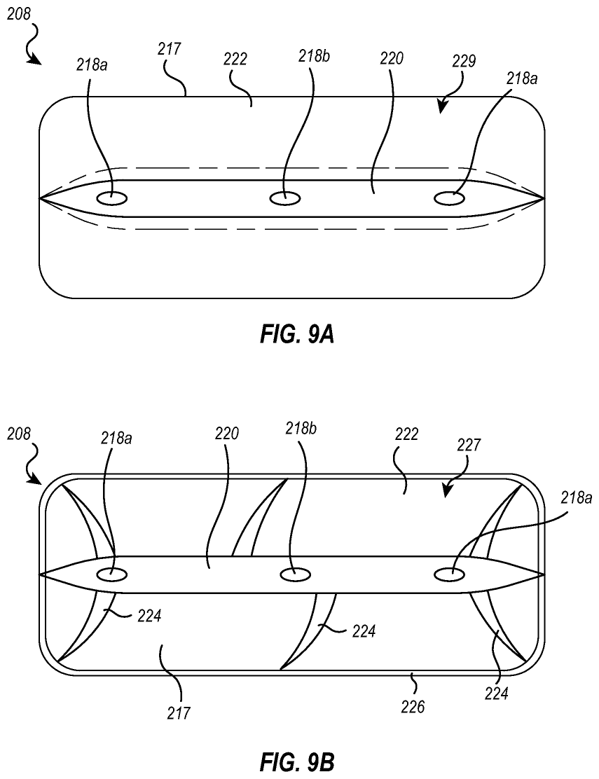

FIGS. 9A-9B illustrate another embodiment of an anchor 208. FIG. 9A illustrates the lumen-facing side 229 while FIG. 9B illustrates the intima-facing side 227. The anchor 208 can include an elongate body 217 having a flexible member or membrane 222 and a centrally-located raised keel 220 spanning a length of the elongate body 217. The elongate shape of the anchor 208 is modified to maximize the surface area of the anchor 208. In this depiction, the number of ribs 224 is reduced which may increase compliance of the anchor 208 to the vessel wall 14. The anchor 208 can also have a raised edge 226 running the perimeter of the elongate body 217. One or more holes 218a, 218b in the keel 220 provide points through which a suture 106 can be threaded to attach the anchor 208 to extravascular components of the closure device 100.

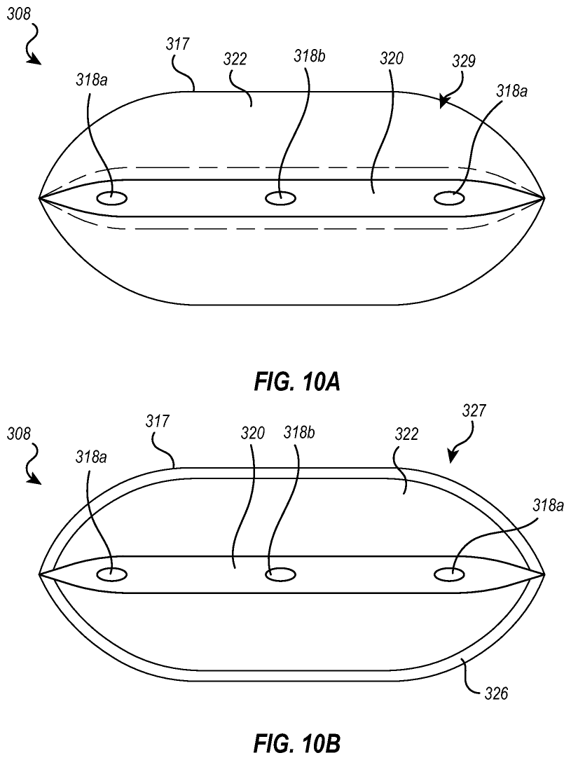

FIGS. 10A-10B, illustrate another embodiment of an anchor 308. FIG. 10A illustrates the lumen-facing side 329 and FIG. 10B illustrates the intima-facing side 327. The anchor 308 can include an elongate body 317 having a flexible membrane and a raised keel 320 located on and spanning the length of a central axis of the elongate body 317. The keel 320 can include one or more holes 318 through which suture(s) 106 can be threaded. In this embodiment, the ribs (124, 224) are omitted to permit maximum flexibility of the anchor 308. The raised edge 326 running the perimeter of the intima side of the anchor 308 can impart the anchor 308 with requisite structural integrity to maintain the shape of the anchor 308 when positioned on the vessel wall 14.

### Method of Closure Device Insertion

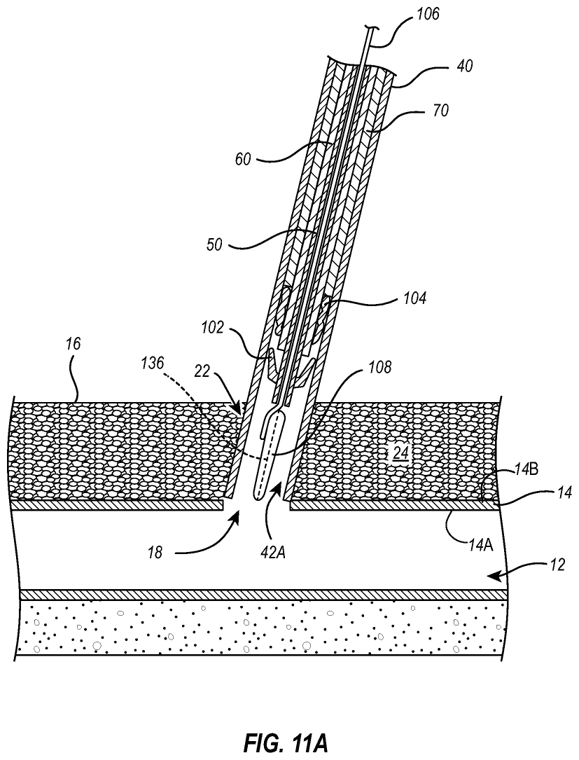

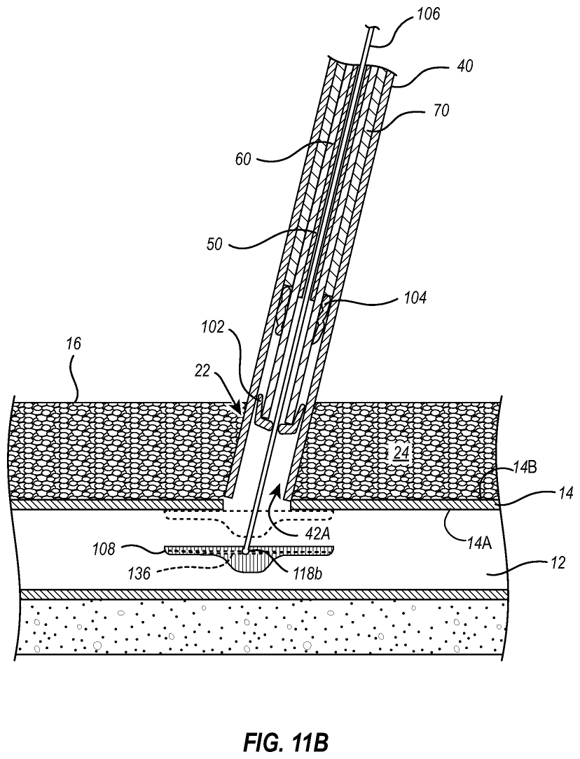

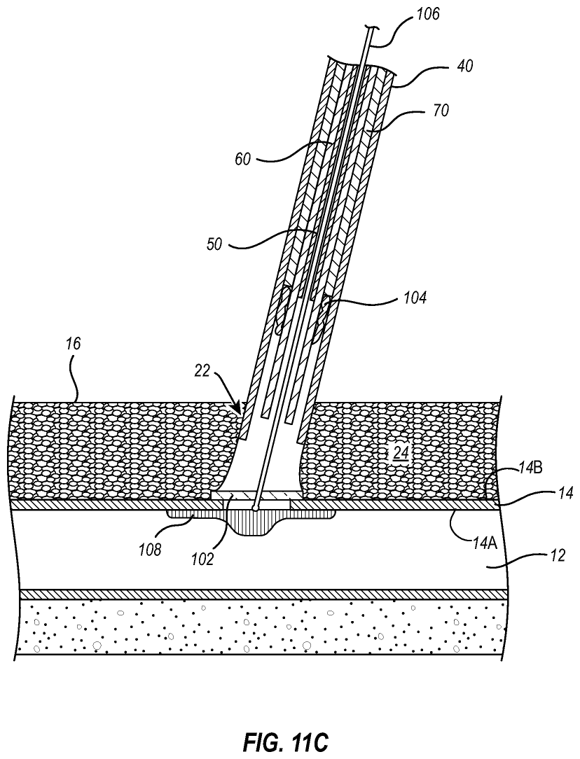

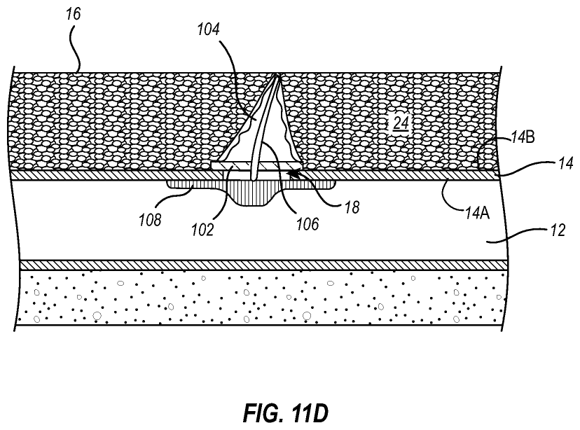

Reference is now made to FIG. 11A, which illustrates a step in the process of deploying the anchor 108. As shown in FIG. 11A, the delivery sheath 40 can be positioned to move the distal end 42A of the outer housing 42 through an access tract 22 defined in tissue 24 and into proximity with a lumen 12 and a puncture or access site 18 defined in a vessel wall 14. The distal end 42A of the delivery sheath 40 is advanced into the lumen 12 until pulsating blood is visually observed from a proximally positioned blood outlet port 49 (see FIG. 1A) of a bleed back or blood marker lumen formed in a wall of the delivery sheath 40. Alternatively, the blood outlet port 49 can be formed by a separated bleed back tube formed either interiorly or externally of the delivery sheath 40. The blood inlet port 47 (see FIG. 1A) is in fluid communication with the blood outlet port 49 and is disposed toward the distal end 42A of the delivery sheath 40.

Once blood flow is observed, the actuator 50 can be manipulated as described above to cause the anchor 108 to be pushed out of the distal end 42A of the outer housing 42. Alternatively, the actuator 60 may push the cap 102 which may, in turn, push the anchor 108 distally relative to the outer housing 42 (as shown in FIG. 11B), thereby deploying the anchor 108 from the distal end 42A of the outer housing 42. In such a case the actuator 50 can optionally be omitted.

In one embodiment, once deployed, the anchor 108 can be configured to rotate or be rotated from a first orientation to a second orientation. In the first orientation, the major axis 136 of the anchor 108 is at a small angle or generally parallel with the outer housing 42 and generally perpendicular to the vessel wall 14, as shown in FIG. 11A. In the second orientation, the major axis 136 of the anchor 108 is generally parallel with the lumen 12 and at a greater angle or generally perpendicular to the delivery sheath 40 as shown in FIG. 11B.

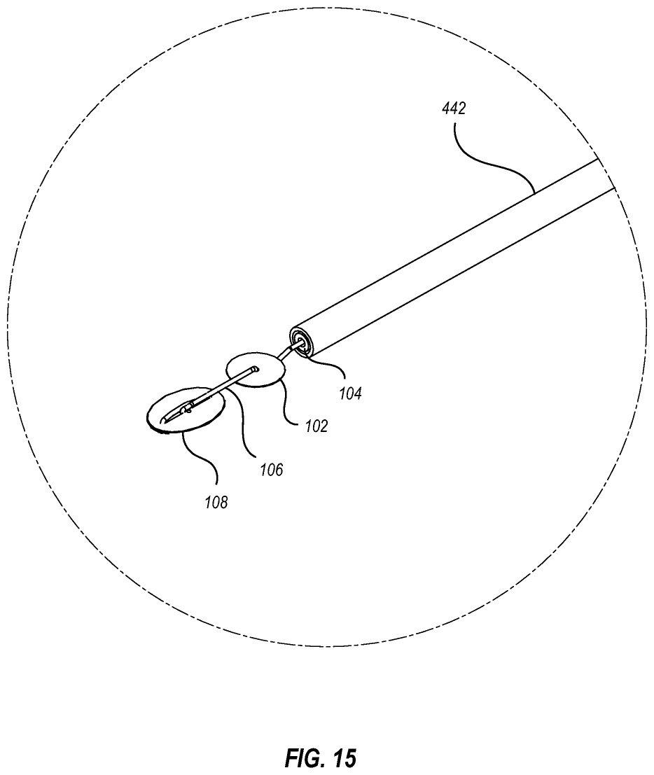

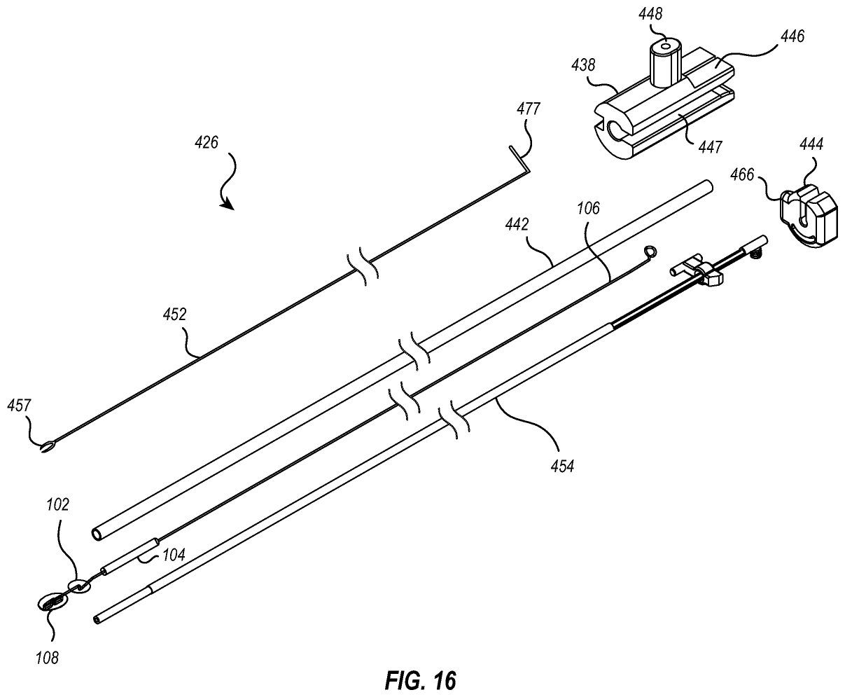

In particular, as shown in FIG. 11B, once the anchor 108 is pushed from the distal end 42A of the outer housing 42, the anchor 108 can rotate or be rotated to the second orientation. Such rotation may occur via tension applied to/by the suture element 106 to the anchor 108 by way of the central or middle hole 118b (see FIGS. 7C-7F). The anchor 108 can then be drawn in the proximal direction to secure the anchor 108 against the inner surface 14A of the vessel wall 14, as illustrated in phantom in FIG. 11B. While the suture 106 is illustrated extending proximally within the lumen of the actuator 50 in FIG. 11A and FIG. 11B, when the actuators are non-coaxial, such as illustrated in FIGS. 15 and 16, the suture 106 need not extend within a lumen of the actuator 50 and actuator 50 need not include a lumen. The suture 106 can extend within any lumen of the delivery system 30, such as illustrated in solid and dashed schematic representations of the suture 106 in FIG. 1F.

With the anchor 108 deployed and positioned against the inner surface of the vessel wall 14, and the delivery sheath 40 partially retracted into the access tract 22, the actuator 60 may then deploy the cap 102 on top of the puncture site 18, between the vessel wall 14 and the tissue 24 through which the tract 22 is formed. In particular, as shown in FIG. 11C, the actuator 60 can be advanced distally, the delivery sheath 40 can be drawn proximally, and/or some combination of such movements can be used to move the cap 102 distally out of the outer housing 42 and into contact with the proximal side or outer surface 14B of the vessel wall 14 adjacent the puncture 18. The vessel wall 14 is positioned between (i.e., sandwiched between) the anchor 108 and the cap 102, with the cap 102 positioned on the outer surface of the access site 18 and “locked” in place as a result of an interference fit created by the thick suture portion 112. Thus, the cap 102 can be positioned to reduce or stop the flow of fluid out of the tract 22 by covering the puncture site 18 and/or obstructing the tract 22.

To verify that blood flow is reduced or stopped, the practitioner can view blood flow from the blood outlet port 49 (FIG. 1A) and determine a degree of hemostasis. A continued degree of blood flow from the blood outlet port 49 (FIG. 1A) may indicate that hemostasis has not been adequately achieved and indicate to the practitioner to continue positioning the cap 102 against the tissue to provide improved hemostasis. Alternatively, blood flow can be observed by maintaining one or more of the valves or seals 55, 65, 75 of the actuators 50, 60, 70, or the one or more valves or seals 45 of the delivery sheath 40, in an open position to allow blood to flow from an end of one or more of the actuators 50, 60, 70 or the delivery sheath 40. In one particular configuration, the actuator 60 can include an enlarged portion that maintains the valve or seal 45 of the delivery sheath 40 open so that blood exits from the end of the lumen when hemostasis has not been achieved. As with the blood flow from the blood outlet port 49 (FIG. 1A), a continued degree of blood flow from the end of one or more of the actuators 50, 60, or 70 or delivery sheath 40 may indicate that hemostasis has not been adequately achieved and indicate to the practitioner to continue positioning the cap 102 against the tissue to adequately achieve hemostasis. Retracting the enlarged portion of the actuator 60 away from or through the one or more valves or seals allows the valves or seals (e.g., 45, 55, 65, 75) to close following advancement of the cap 102 towards the anchor 108 to achieve hemostasis and reduce blood flow.

Returning to FIG. 11C, advancing the cap 102 towards the anchor 108 aids with stabilizing the tissue 24 around the puncture site 18 in order to facilitate closure of the puncture site 18. In particular, once the anchor 108 and the cap 102 are deployed, tension can be applied to the suture 106 to secure the anchor 108 against the inner surface 14A of the vessel wall 14 while the actuator 60 advances the cap 102 distally toward the outer surface 14B of the vessel wall. In one example, a suture lock (not shown) can be utilized to help maintain the tension in the suture element 106. The combination of the forces exerted by the anchor 108 and the cap 102 on the vessel wall 14 provides a compressive force on the tissue 24 near the puncture site 18, i.e., sandwiching the tissue 24 between the anchor 108 and the cap 102. The tension applied to the suture 106 can range from about 1 pound-force (lbf.) to about 16 lbf., from about 1 lbf. to about 8 lbf., from about 2 lbf. to about 6 lbf., or about 2.5 lbf.

Because the anchor 108 is formed of a resilient pliable material and the cap 102 can be formed of elastomeric materials (such as bioabsorbable polymers, bioabsorbable elastomers, etc.), the material properties allow the anchor 108 and the cap 102 to accommodate applied forces without fracturing, buckling, snapping, tearing, or otherwise failing. The suture 106 can also include a visual indicator to show the user when the cap 102 has reached the proper depth, i.e. the cap 102 has reached the vessel wall 14. If too much force is applied, this may cause the suture 106 to break, however, due to the lack of a knot or other static element maintaining the cap 102 in a fixed position, the cap 102 and the anchor 108 will not over-tension. Because of this feature, the practitioner does not have to worry about the degree of force applied.

Placement of the cap 102 also pushes the tissue 24 in a transverse direction in relation to an axis of the tract 22. This increases a space for subsequent delivery of the sealant 104, and increases a surface area of the vessel wall 14 and the cap 102 that can receive the sealant 104. By so doing, the efficacy of puncture site 18 closure is enhanced.

Optionally, in a configuration where the actuator 60 can deploy both the anchor 108 and the cap 102, the actuator 60 can remain in continuous contact with the cap 102 throughout the deployment process. Such a configuration can allow the anchor 108 and/or cap 102 to be deployed by advancing the actuator 60 in a single direction. By facilitating deployment of the anchor 108 and cap 102 using one-way movement of the actuator 60, and by utilizing a single actuator, the delivery system can be used to quickly and easily deploy the anchor 108 and/or cap 102 and sealant 104.

Optionally, in a configuration where the actuator 60 can both deploy the cap 102 and advance the sealant 104 towards the cap 102, the distal movement of the actuator 60 advances the sealant 104 towards the cap 102, with a subsequent proximal movement of actuator 60 releasing the sealant 104 from within the actuator 60. In this configuration, the actuator 70 is optionally omitted.

Returning to the illustrated configuration, once the cap 102 is placed, the sealant 104 can be deployed from the delivery sheath 40 by proximally withdrawing the delivery sheath 40 and, optionally, the actuator 60, and distally advancing the actuator 70. The sealant 104 can be deployed by some combination of one or more of such movements to advance or release the sealant 104 from the outer housing 42 and into contact with the outer surface 14B of the vessel wall 14 and the cap 102. As the delivery sheath 40 is proximally moved or retracted, and/or the actuator 60 is proximally moved or retracted, the sealant 104 is exposed to bodily fluids to activate the sealant 104, as illustrated in FIG. 11D. The activated sealant 104 can act as an adhesive to secure the cap 102 in place as well as reinforce the hemostatic effect of the cap 102 by coagulating the access tract 22 and preventing leakage. It can be advantageous to have the sealant 104 as close to the surface of the skin as possible to mitigate any potential bleeding.

While the sealant is activated, such as can occur in from about 15 seconds to about 5 minutes, from about 30 seconds to about 4 minutes, from about 1 minute to about 3 minutes, from about 15 seconds to about 1 minute, from about 15 seconds to about 45 seconds, the practitioner can view blood flow, if any, from the blood outlet port 49 (FIG. 1A) and determine a degree of hemostasis. Based upon the force applied to the cap 102 to seal the puncture site 18, the cap 102 can seal or substantially seal the puncture site 18 resulting in the sealant 104 being used to limit tissue oozing around the cap 102 and from the tissue tract 22. The sealant 104 also provides secondary securement of the cap 102 in relation to the suture 106 and the puncture site 18. Stated another way, primary closure of the puncture site 18 can be achieved through the seal provided by the anchor and cap, while the sealant 104 provides secondary sealing and/or stopping of tract ooze.

If there is, however, a continued degree of blood flow from the blood outlet port 49 (FIG. 1A), the practitioner can further manipulate the actuators and anchor 108 to tighten the cap 102 on the suture 106 or, optionally, wait for the sealant 104 to sufficiently activate to reduce or eliminate blood flow to the practitioner's preferences. More generally, with the cap 102 and sealant 104 combination, dry close may be achieved within seconds of activating the sealant 104. Practitioners can also compress the area with a gauze to express out any blood and then check for hemostasis. While illustrative times to hemostasis are provided, time to hemostasis can be impacted by or correlated to anticoagulant agents given to the patient. With the combination of the cap 102 and proximal sealant 104, hemostasis may be achieved faster than with sealant 104 alone.

Whether complete or substantially complete hemostasis occurs from the cap 102, or a combination of the cap 102 and the sealant 104, after hemostasis is achieved, the suture 106 can be trimmed by pushing down on the skin 16 while tensioning the suture 106 and using a suture trimming device (not shown), such as a scalpel or other suture trimming device, to trim the suture 106 as close to the skin as possible. Once the skin is released, the suture 106 will sit well below the surface of the skin, as shown in FIG. 11D.

While reference has been made to the anchor 108 (208, 308) remaining in the blood vessel and being degraded, absorbed, or resorbed by the patient's body, it will be understood that in other configurations, the anchor 108 may be deployed and subsequently removed once sufficient closure of the puncture site 18 has occurred. In such a case, the anchor 108 is “temporarily” deployed and the other portions of the closure element 100, such as the cap 102 with the adhesive layer 128 (see FIGS. 3C and 3D) and the sealant 104 described herein, can be used to close the access site following removal of the anchor 108. The cap 102 with the adhesive layer 128 may or may not cooperate with a suture 106 and lock onto a suture 106 that is optionally attached to the anchor 108. The cap 102 is maintained in place against the vessel wall 14 by the adhesive layer 128 and, optionally, the sealant 104, with the sealant 104 reducing or eliminating any tissue tract 22 oozing. Delivery of the temporary anchor 108, the cap 102, and the sealant 104 in this alternate configuration can be performed using the delivery systems and devices described herein, while accommodating removal of the anchor 108 by proximally drawing on the suture 106, or another anchor actuator, to remove the anchor 108. The anchor 108 may optionally pass through the lumen 110 of the cap 102, with the body of the cap 102 being sufficiently resilient/elastic to return to a closed state to close the puncture site 18. Alternatively, the anchor 108 can be withdrawn past a side of the cap 102, with the cap 102 having sufficient resiliency to temporarily deform and return to a state to seal against the outer surface 14B of the vessel wall 14.

### Handle Assembly Vessel Closure Delivery System

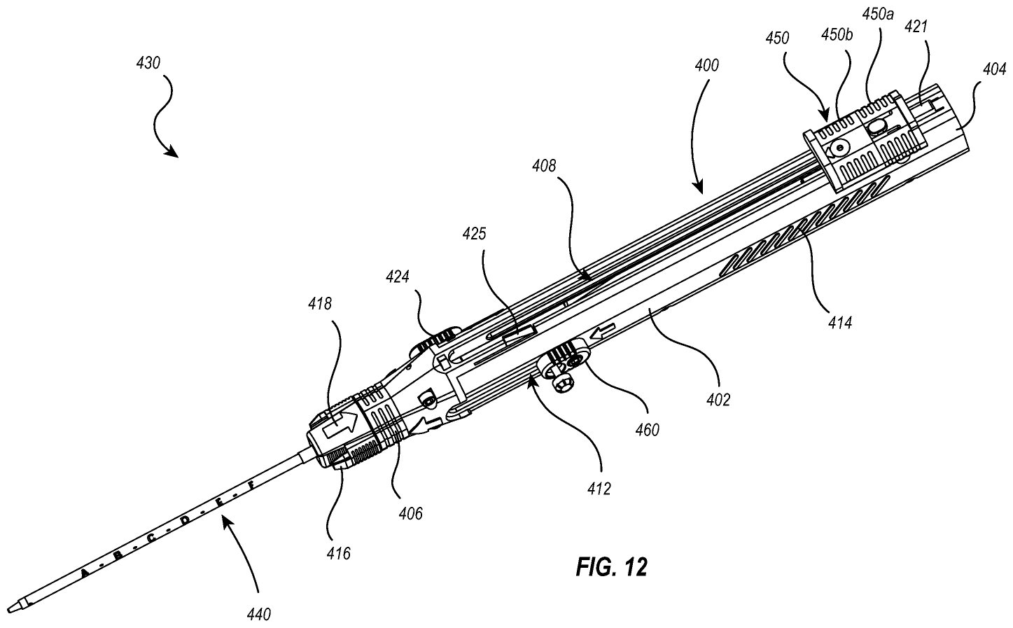

FIGS. 12-23B illustrate a delivery system and method of inserting a closure device of the type described herein. The delivery system 430 can include a handle assembly 400 and a delivery sheath 440. The handle assembly 400 can be configured to be selectively attached to a delivery sheath 440 (similar to delivery sheath 40 of FIGS. 1A-1F). Once attached to the delivery sheath 440, the handle assembly 400 can be used to insert a closure device, such as, for example, closure device 100.

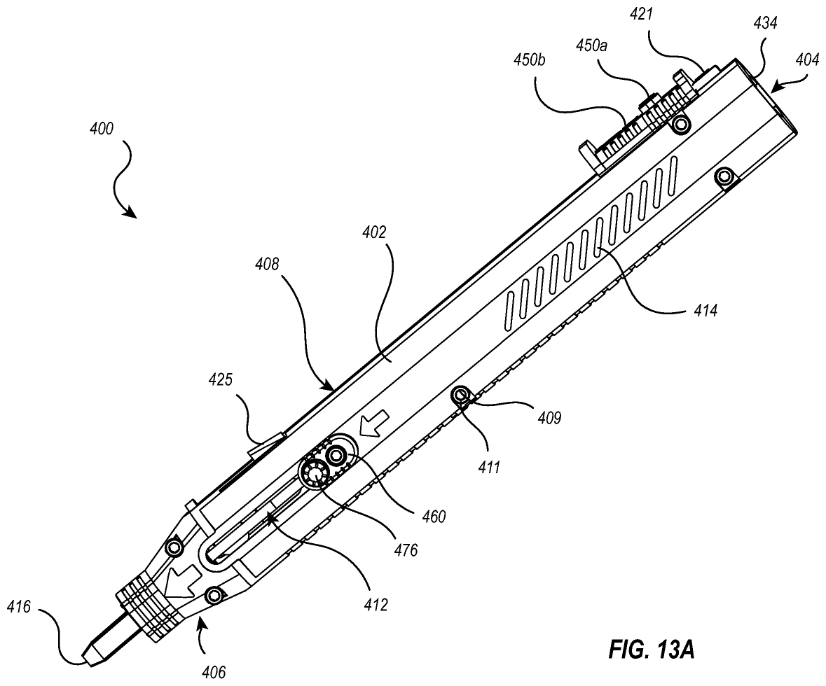

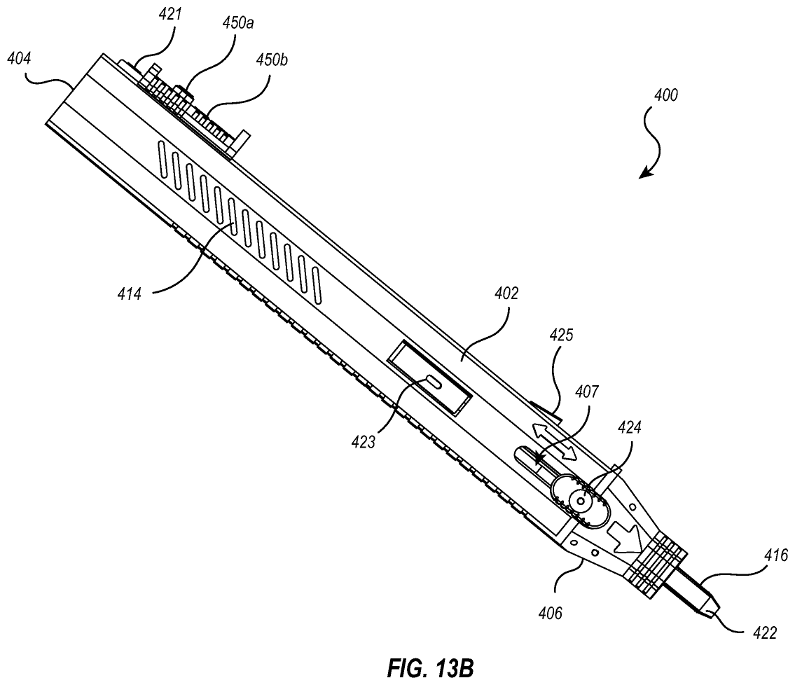

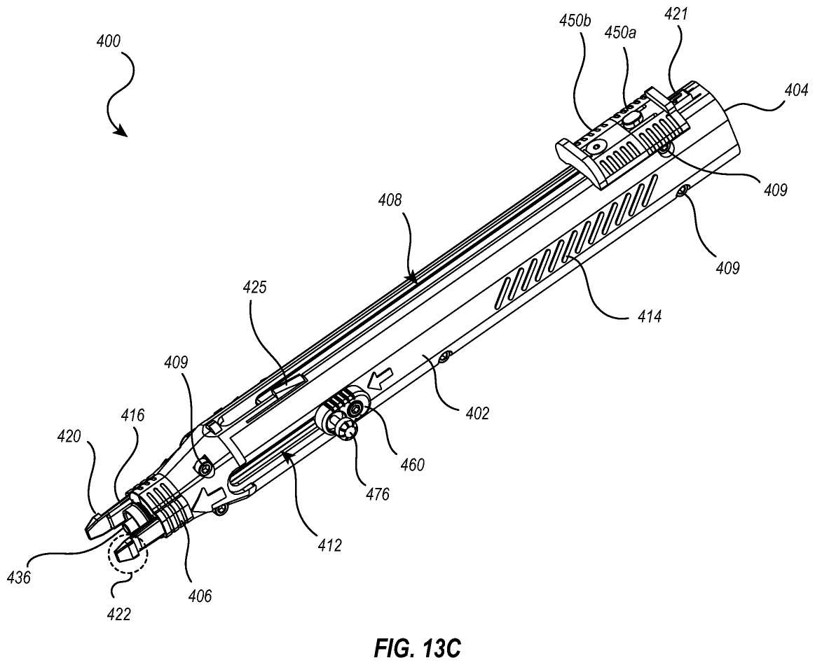

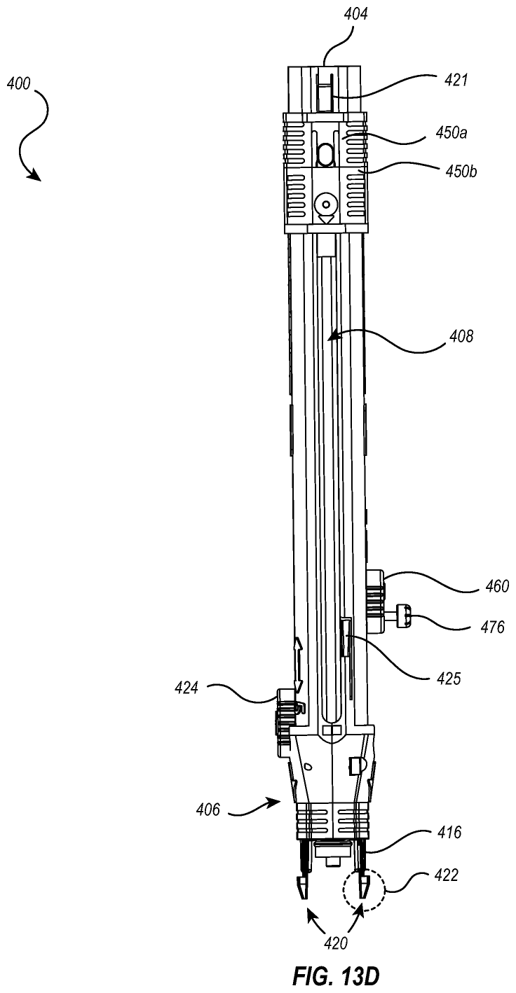

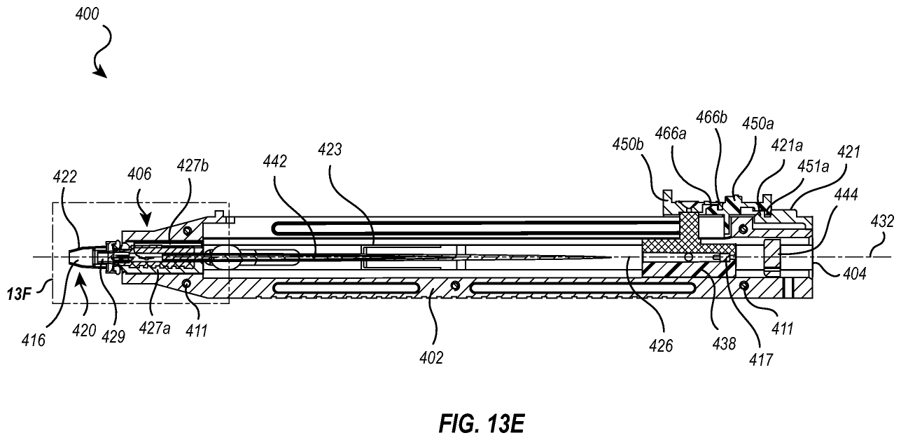

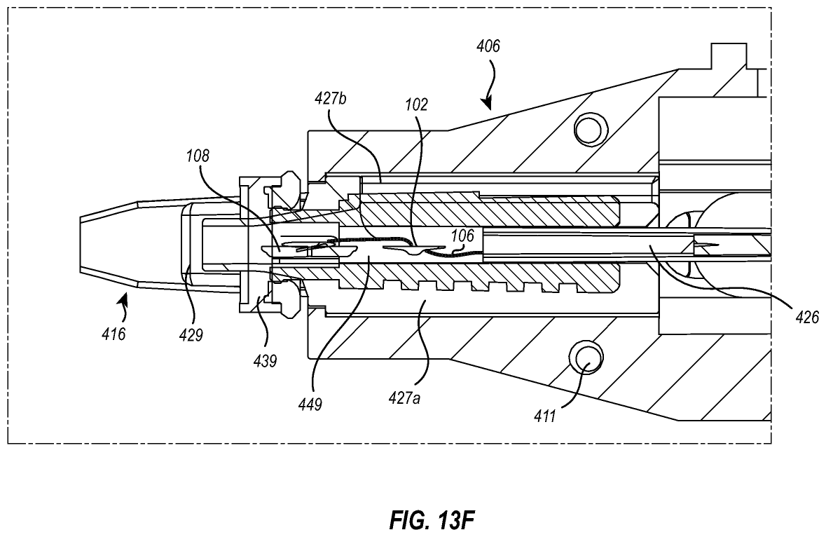

As shown in FIGS. 13A-13E, the handle assembly 400 can include a handle body 402 having a proximal end 404 and a distal end 406, one or more actuators (such as slider 450), and an elongate opening 408 configured to provide a track for the slider 450. The slider 450 can be configured to slide along the elongate opening 408 when engaged by a practitioner and be selectively locked in place by a locking assembly 425. Sliding or moving the slider 450 along the elongate opening 408 can deploy the closure device 100. The handle assembly 400 can also include a second slide (e.g., a cap slide) 460 configured in a second elongate opening 412 on the handle body 402. Engagement of the slider 450 can deploy the anchor 108, and then engagement of the cap slide 460 can deploy the cap 102.

In other embodiments, the handle assembly 400 may have only one actuator element, such as slider 450, which when engaged can subsequently deploy the anchor 108 and cap 102 without the need for a second slide. Any number of slides or other actuators could be provided.

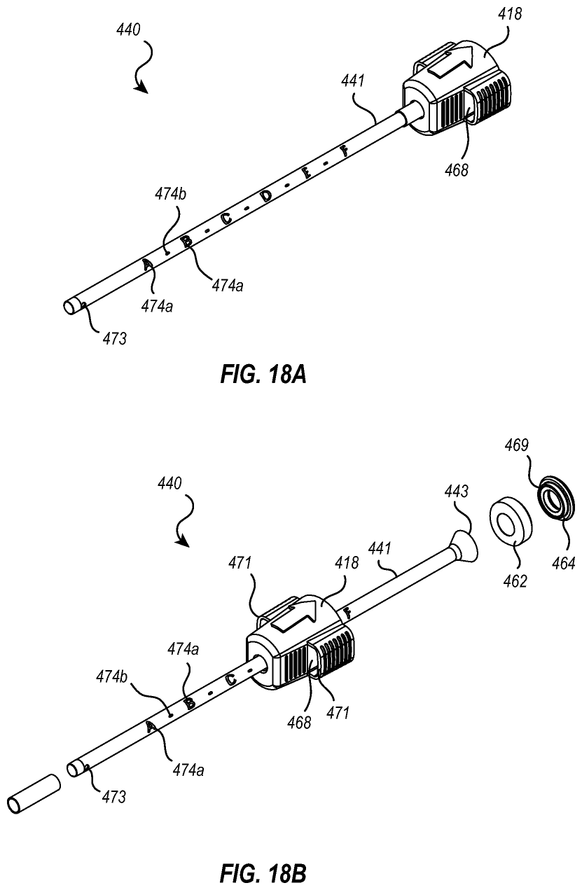

In some embodiments, such as the embodiment shown in the drawing, the handle body 402 can include one or more textured portions 414 to improve a practitioner's grip on the handle assembly 400. The handle assembly 400 can further include a connecting member 416 located at the distal end 406 of the handle body 402. The connecting member 416 can be configured to be selectively attached to and removeable from a delivery sheath 440. The connecting member 416 can also be configured to attach to a sheath hub 418 of the delivery sheath 440 (see FIGS. 18A-18B). The connecting member 416, as shown in FIGS. 13A-13F, comprises a set of locking members 420, each having hooked ends 422. The locking members 420 can be configured to selectively attach to the sheath hub 418 of the delivery sheath 440. The sheath hub 418 attaches the handle assembly 400 to the delivery sheath 440 to form the delivery system 430.

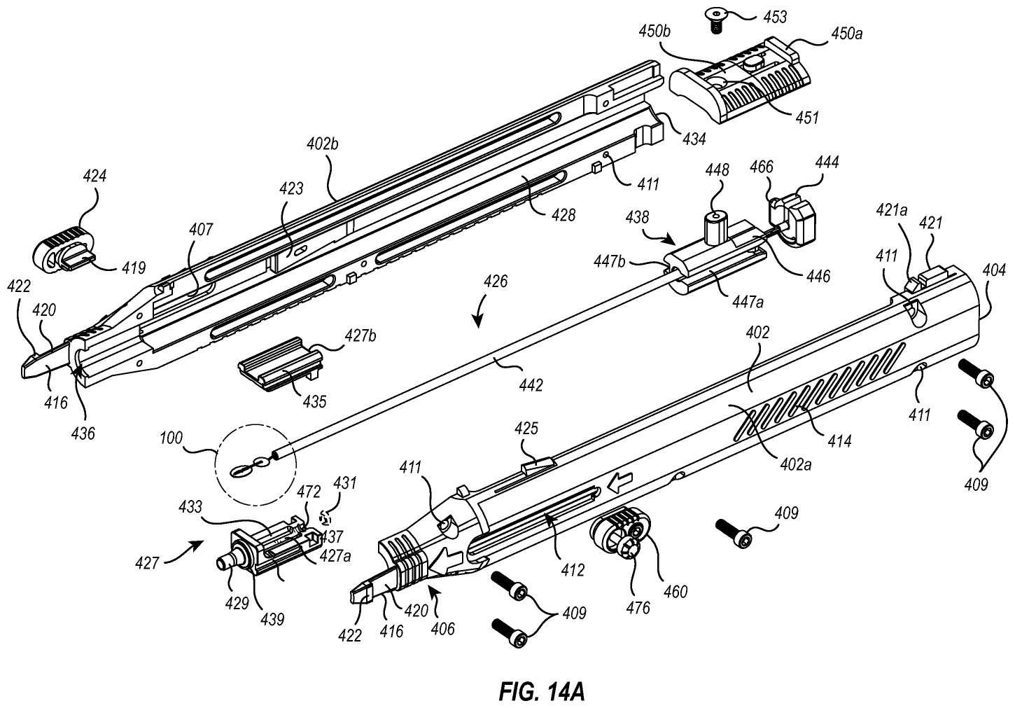

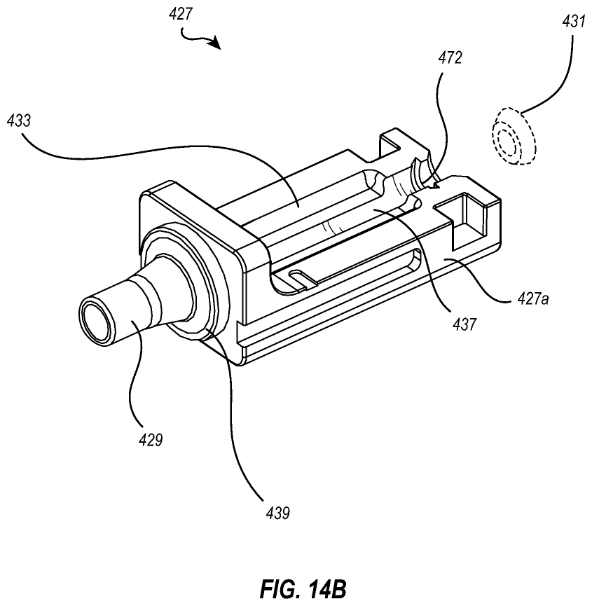

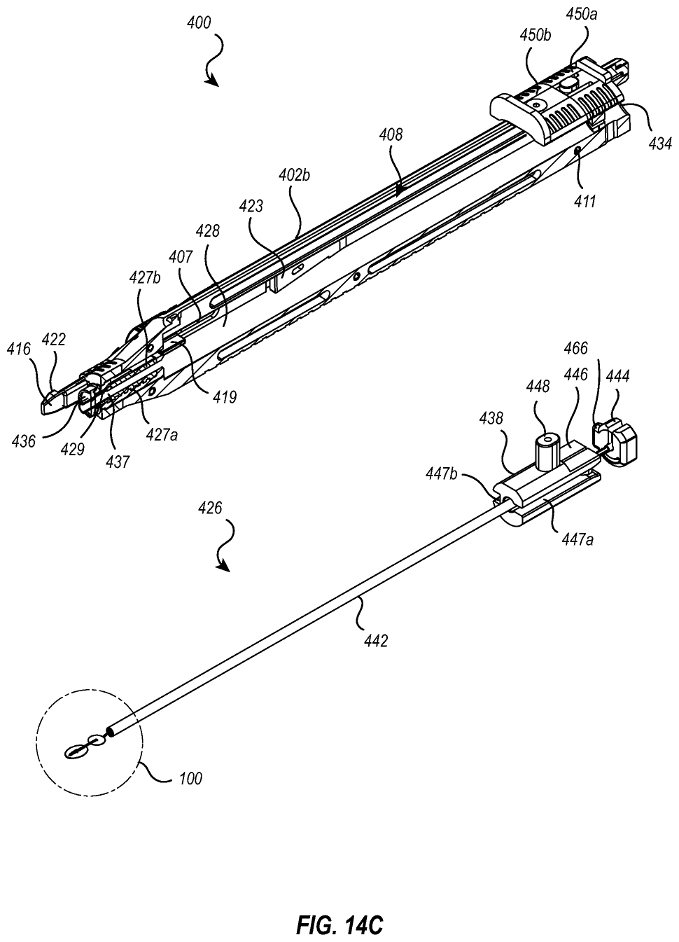

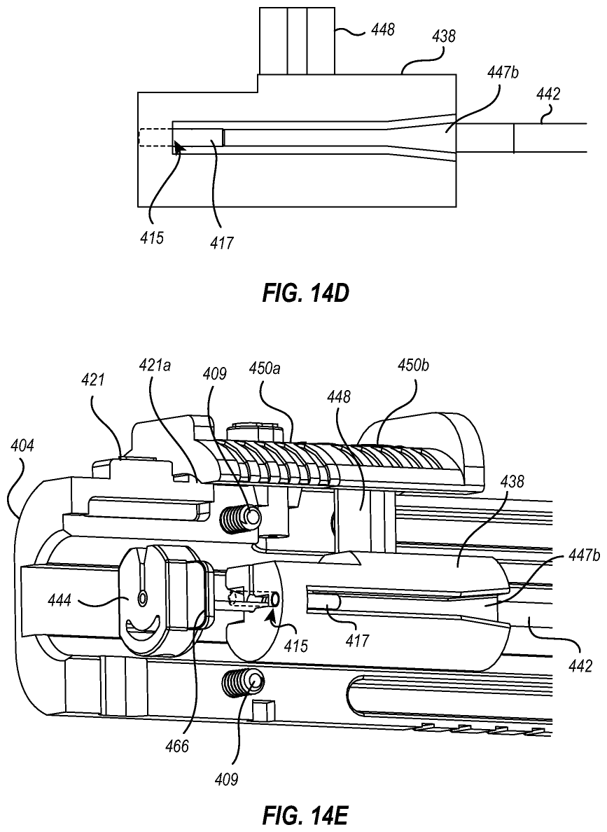

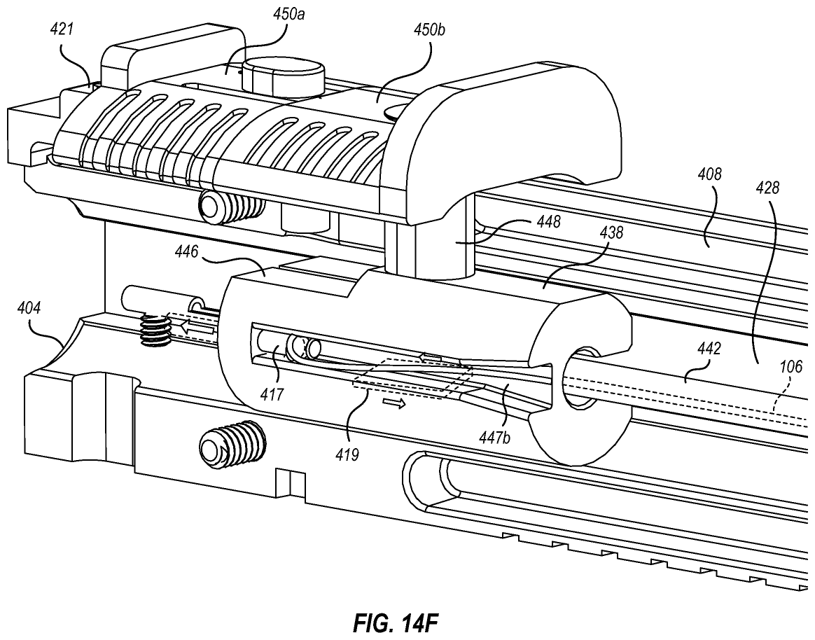

As shown in FIG. 13B, the handle assembly 400 can also include a release button 424 which can release the suture 106 from the system 430 once the closure device 100 is placed at a desired location. Engagement of the release button 424 can release (e.g., separate or disengage) the delivery system 430 from the implanted closure device 100. The release button 424 can include an engagement element, such as release button fin 419 (see FIG. 14A). The release button fin 419 is configured to fit within release groove 407 and slide within the length of the groove 407 to release the suture 106 of the closure device 100 from the handle assembly 400. In other embodiments, the functions of the release button 424 may be incorporated into one or more actuator elements such as slider 450 and/or cap slide 460.

FIG. 13E illustrates a cross-sectional view of the handle assembly 400. As shown in the FIGS. 13A-13E, slider 450 can include a first portion 450a and a second portion 450b. As best seen in FIG. 13E, the portions 450a, 450b can be selectively connected together by interlocking ends 466a, 466b. A proximal locking assembly 421 can engage slider 450 to “lock” slider 450 at the proximal end 404 of the handle assembly 400. For instance, complementary structures on the proximal locking assembly 421 and portion 450a of the slider 450 can engage to limit movement of the slider 450. Depressing the proximal locking assembly 421 detaches or separates the complementary structure 421a from the complementary structure 451a, allowing the slider 450 to move distally. The portions 450a, 450b, their interlocking ends 466a, 466b, and proximal locking assembly 421 can be made of a resilient material, such as a flexible plastic, to allow the components to flex when depressed by a practitioner. For example, the practitioner can depress the proximal locking assembly 421 to release the slider 450 and allow the slider 450 to slide along elongate groove 408. The proximal locking assembly 421 can be formed with the handle body 402, such as having a living hinge connection with the handle body 402 or can be a separated mechanism connected or mounted to the handle body 402.

FIGS. 14A and 14C illustrate an exploded view of the handle assembly 400.

The handle body 402 can comprise a first side 402a and a second side 402b, which when assembled together form the lumen 428 of the handle body 402. The first side 402a and second side 402b can be assembled together to form the handle body 402 by using fasteners, such as screws 409 inserted into corresponding bores 411. Other mechanisms for attachment can of course also or alternatively be used. The handle body 402 can be configured to house an implant assembly 426. The implant assembly 426 (discussed more fully below) includes a tamper tube 442, a carriage 438, a stopper 444, and the closure device 100.

The handle body 402 can also house a chamber assembly 427 having a chamber body 427a and a chamber cap 427b as shown in FIGS. 13F and 14B, which can be disposed at the distal end 406 of the handle body 402. While the chamber assembly 427 is illustrated as two pieces, it will be understood that the chamber assembly 427 can utilize less or more pieces to form an assembly that can provide the functions described herein. The chamber assembly 427 can also be formed separately from the handle body 402, as shown, though in other embodiments, the chamber assembly 427 may be integrally formed within the handle body 402. The chamber assembly 427, and in particular the chamber body 427a, can align with the lumen 428 and the distal opening 436 to form a channel 437 through which the implant assembly 426 can deploy the closure device 100.

The chamber assembly 427 can include a chamber body 427a with a nozzle 429 and a nozzle ring 439. The nozzle 429 and ring 439 can be shaped to interface with the delivery sheath 440 and form a fluid-tight seal between the handle assembly 400 and the delivery sheath 440. The closure device 100 of the implant assembly 426 can be deployed from the lumen 428 through the channel 437 and then out of the nozzle 429 of the chamber assembly 427, such as from the chamber body 427a, into the delivery sheath 440. In some embodiments, the chamber assembly 427 can include a valve 431. The valve 431 can be a one-way valve, preventing fluids from entering the lumen 428 of the handle body 402. The valve 431 can be seated within a valve notch 472 at the proximal end of the chamber body 427a. The chamber body 427a can also include a plateau 433.

The chamber assembly 427, as shown in FIG. 14A, includes a chamber cap 427b. The chamber cap 427b can be situated on top of the chamber 427 in the distal end 406 of the handle body 402. The chamber cap 427b can help form the channel 437 and can include one or more positioning elements 435 which can retain the chamber cap 427b in the correct orientation and location in the handle body 402. The chamber body 427a and the chamber cap 427b, when connected or coupled together, form a cavity 449 to receive the closure device 100, as illustrated in FIG. 13F. The cavity 449 communicates with, and forms part of the lumen 428.

The implant assembly 426 is contained within the handle body 402. The implant assembly 426 houses the closure device 100 and other elements required to position the closure device 100. The implant assembly 426 can be configured to be positioned within the lumen 428 of the handle body 402. The lumen 428 can extend from a proximal opening 434 at the proximal end 404, along a longitudinal axis 432, and terminate at a distal opening 436 at the distal end 406 of the handle body 402. The implant assembly 426 can be situated within the lumen 428 such that it can be in mechanical communication with elements of the handle body (i.e., slider 450 and cap slide 460). The implant assembly 426 (shown in detail in FIGS. 14A, 14C-14F, 15 and 16) includes a closure device such as closure device 100, a tamper tube 442, a carriage 438, and a stopper 444. The carriage 438 can comprise a body 446 having a protrusion 448 providing for mechanical interface between the slider 450 (on the external side of the handle body 402) and the body 446 situated on the tamper tube 442 within the lumen 428 of the handle body 402. The carriage 438 can also include a groove 447a configured to receive nested elements (e.g., tamper tube 442, closure device 100, tube 454, and push wire 452) of the implant assembly 426. The carriage 438 can also include a suture groove 447b, which can allow for mechanical communication between the implant assembly 426 and the handle body 402, to facilitate release of suture 106 from the implant assembly 426 and the handle body 402.

The stopper 444 can include a stopper elbow 466 configured to engage with interior locking mechanism 423. When the stopper 444 is moved in a distal direction (i.e., towards the distal end 406 of the handle body 402), the stopper 444 will pass the interior locking assembly 423. Once past the interior locking assembly 423, the stopper elbow 466 can engage the interior locking assembly 423, preventing the stopper 444 from moving in a proximal direction. The stopper 444 can prevent closure device elements, such as the fluid blocking component 104, from flowing back into the handle assembly 400. The interior locking mechanism 423 can be formed with the handle body 402, such as having a living hinge connection with the handle body 402 or can be a separate mechanism connected or mounted to the handle body 402.

FIGS. 14D and 14E illustrate detailed views of carriage 438 of the implant assembly 426. As discussed above, the carriage 438 can include one or more structures configured to engage with exterior elements of the handle body 402, to control insertion and placement of the closure device 100 and disengagement of the closure device 100 from the delivery system 430. For example, the suture groove 447b of carriage 438 can house a pin 417 positioned within a bore 415.

As shown in FIG. 14F, the suture 106 can be looped around the pin 417 during assembly. A friction fit of the suture 106 between the pin 417 and the suture groove 447b can retain the suture 106 within the carriage 438 during insertion of the closure device 100. After the closure device 100 is deployed around the blood vessel, the delivery system 430 is decoupled from the closure device 100 by releasing the suture 106 from the carriage 438. The release button 424 (not shown in FIG. 14F) is slid in a proximal direction towards the pin 417, causing the release button fin 419 to push the pin 417 into bore 415. When the pin 417 is pushed into the bore 415, the suture 106 is released from the pin 417, effectively releasing the suture 106 and closure device 100 from the delivery system 430.

The tamper tube 442 can contain the suture 106, which can be threaded therethrough. As shown in FIG. 16, the tamper tube 442 can also contain a push wire 452 and a support tube 454. The distal tip 457 of the push wire 452 can have a forked or pronged shape to help push the closure device 100 out of the delivery system (the forked or pronged shape can be inline or angularly orientated in relation to the push wire 452). Other shapes for tip 457 are of course also possible. A proximal end of the push wire 452 includes a push wire bend 477 that mounts to the portion 450a so that the push wire 452 can be moved through movement of the portion 450a. The support tube 454 can be used to tamp the cap 102 of the closure device 100 after the anchor 108 is positioned. The stopper 444 can prevent the implant assembly 426 from sliding out of the distal opening 436 of the handle body 402.

The closure device 100, as discussed herein, can comprise an anchor 108, a cap 102, and a fluid-blocking component or sealant 104 all configured on a suture 106. The sealant 104 can be an active biologic material, such as polyethylene glycol (PEG), fibrin sealants, copolymer of glucosamine and N-acetyl glucosamine, dextran (a complex branched glucan (a polysaccharide derived from condensation of glucose), polypeptide adhesive structures, adhesive protein containing L-3,4-dihydroxyphenylalanine (L-DOPA), adhesive protein containing DOPA and phosphoserine, collagen, polyacrylic acid, cross-linked with allyl sucrose or allyl pentaerythritol, polyacrylic acid, cross-linked with divinyl glycol, Acrylic resinous polymer composed of methyl-2-cynoacrylate units, or another fully bioabsorbable sealant-type material that could be optionally incorporated into a shaped, flexible substrate. The sealant material is activated by fluids present in the patient's tissue tract, such as blood or other fluids, and can be protectively stored inside the sheath/actuators or a chamber of the delivery device until positioned directly on top of the cap 102.

Once advanced into the desired location, the sealant 104 can be exposed to blood or bodily fluids, such as through unsheathing the sealant 104 and positioning the sealant 104 into direct contact with the tissue where it can react. This reaction can cause the sealant 104 to expand, absorbing blood and other fluids, and bonding to surrounding tissue and the cap 102. The sealant 104 can act as a glue, aiding with “locking” the cap 102 in place on a blood vessel, and actively coagulating the entire tissue tract 22. The chemical formulation, quantity, carrier matrix, and dimensions of the sealant 104 can be selected specifically to provide one or more of the functions of locking in place of the sealing component (e.g., cap 102), providing a fast acting and leak-free dry close, and reducing tissue tract oozing.

For instance, the sealant 104 can form a plug having a length of about 1 mm to about 10 mm and can, optionally, be trimmed to a length in the patient along with the suture 106 after deployment. Alternatively, the sealant 104 can extend the full length of the tissue tract 22 and be trimmed to fit the patient. When the sealant 104 is formed of a matrix, the matrix can have an area of about 0.012 square inches to about 0.12 square inches, about 0.12 square inches to 0.6 square inches, or about 0.6 to 1.0 square inches. The matrix material can be thin and flexible such that it can be wrapped around the suture in the delivery system to fit inside a tube for delivery to the implant location. This results in a volume of fluid-blocking component, optionally including a matrix containing a sealant such as PEG or other biocompatible material, of between about 0.004 to about 0.040 cubic inches in volume, about 0.040 to about 0.100 cubic inches, or about 0.100 to about 0.4 cubic inches.

The sealant 104 can be arranged so that it is disposed on the suture 106. The sealant 104, therefore, can be deployed in a flowable composition without a carrier matrix or can be formed as part or with a carrier matrix. For instance, the sealant 104 can be disposed around the suture 106 in a generally cylindrical component, can be bonded to the suture 106 itself, can be bonded to the cap 102, and combinations or modifications thereof. Because the sealant 104 is positioned proximal relative to the cap 102, the sealant 104 can actively coagulate the access tract 22 and, optionally, actively coagulate all of access tract 22 to the surface of the skin 16.

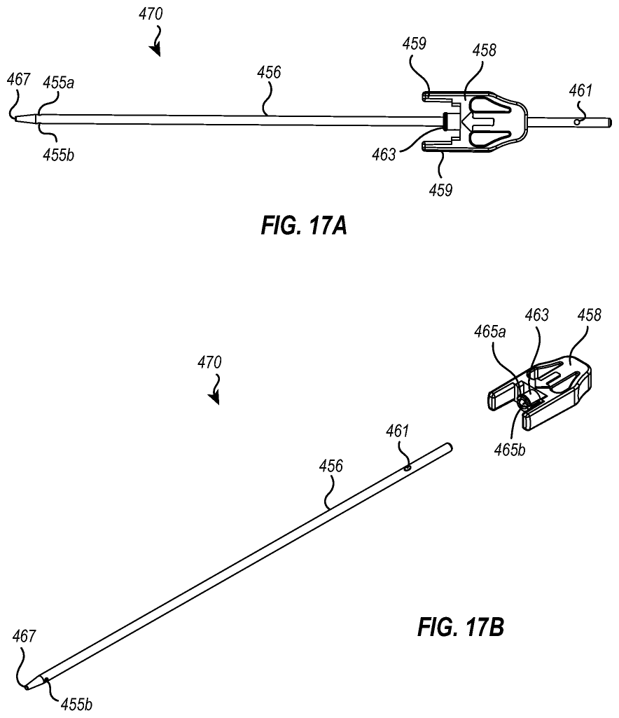

FIGS. 17A and 17B illustrate a dilator assembly 470 having a dilator tube 456 with a dilator hub 458 which can be assembled on the dilator tube 456. The dilator tube 456 can be inserted into the delivery sheath 440 in order to stretch the opening in the skin 16 and access tract 22 to allow for insertion of the delivery sheath 440. The dilator hub 458 can be configured to be selectively attached to and removed from the delivery sheath 440 via the sheath hub 418. The dilator hub 458 can include locking arms 459 which can selectively engage the receiving members 468 of the sheath hub 418, such as through an interference or friction fit. The dilator tube 456 and/or the dilator hub 458 can be formed of biocompatible materials, such as but not limited to nylon, Polyethylene, High Density Polyethylene (HDPE), or other polymeric materials.

The dilator tube 456 includes distal openings 455a, 455b toward a distal end 467, and a proximal opening 461 towards a proximal end. The distal openings 455a, 455b communicate with an internal passageway to form a fluid marker (e.g., blood marker) to aid with positioning the dilator tube 456 within a body lumen. For instance, a fluid from inside a body lumen, such as blood, is permitted to flow through one or both of the distal openings 455a, 455b and through the internal passage and out of the proximal opening 461 to indicate a particular depth of the tube 456. While the distal openings 455a, 455b are illustrated as being positioned on opposing sides of the dilator tube 456, it will be understood that the location and number of openings can vary.

Disposed between the locking arms 459 is a mounting member 463 that aids with mounting the dilator hub 458 to the delivery sheath 440. The mounting member 463 also facilitates connection of the dilator tube 456 to the dilator hub 458. The mounting member 463 can be bifurcated with a first leg 465a and a second leg 465b. The bifurcated structure allows for flexing of the mounting member 463 as it engages with the delivery sheath 440, while the mounting member 463 friction or interference fits within the sheath hub 418.

The delivery sheath 440, shown in FIGS. 18A and 18B, comprises a sheath 441 for receiving the dilator tube 456 and for delivering the dilator tube 456 and the implant assembly 426 through the access tract 22. A sheath hub 418 can be assembled on the sheath 441 in order to allow for the selective attachment of other surgical instruments to the delivery sheath 440, such as dilator tube 456. The sheath hub 418 can include receiving members 468 configured to receive surgical instruments and selectively retain the surgical instruments on the delivery sheath 440, such as but not limited to the locking members 420 of the handle assembly 400 and the locking arms 459 of the dilator hub 458. The receiving member 468 can be channels or passages formed by a wall 471. The proximal end 443 of the sheath 441 can cooperate with a valve 462 to prevent the backflow of fluid into a surgical instrument attached to the delivery sheath 440. The valve 462 is retained within the sheath hub 418 by a valve cap 464, with a strain relief member 469 extending distally from the valve cap 464. One or more of the sheath hub 418, the sheath 441, the valve 462, the valve cap 464, and the strain relief member 469 can be bonded together through an overmold bond technique or otherwise mounted together using a combination of friction or interference fit and adhesives, thermal, chemical, or other bonding techniques.

When the dilator assembly 470 is mounted to the delivery sheath 440, the mounting member 463 passes through the valve cap 464 and the valve 462. With one or more ports 473 aligned with the distal openings 455a, 455b, a fluid pathway is formed to allow for depth determination and location of the delivery sheath 440. Additionally, indicia 474 (e.g., 474a, 474b) are provided on the sheath 441 to provide a depth indication for the delivery sheath 440. For instance, letters, numbers, or other symbols can be used to identify insertion depth. In one configuration, first indica 474a, can be separated by about 1 cm, with a second indica 474b being about 0.5 cm from the adjacent first indicia 474a. It will be understood that one or more second indica 474b can be disposed between adjacent first indica 474a, thereby changing the depth granularity. Additionally, the separation of the first indica 474a can range from about 0.1 cm to about 5 cm, from about 0.25 cm to about 2.5 cm, about 0.5 cm to about 1 cm, less than about 5 cm, less than about 4 cm, less than about 3 cm, less than about 2 cm, less than about 1 cm, less than about 0.5 cm.

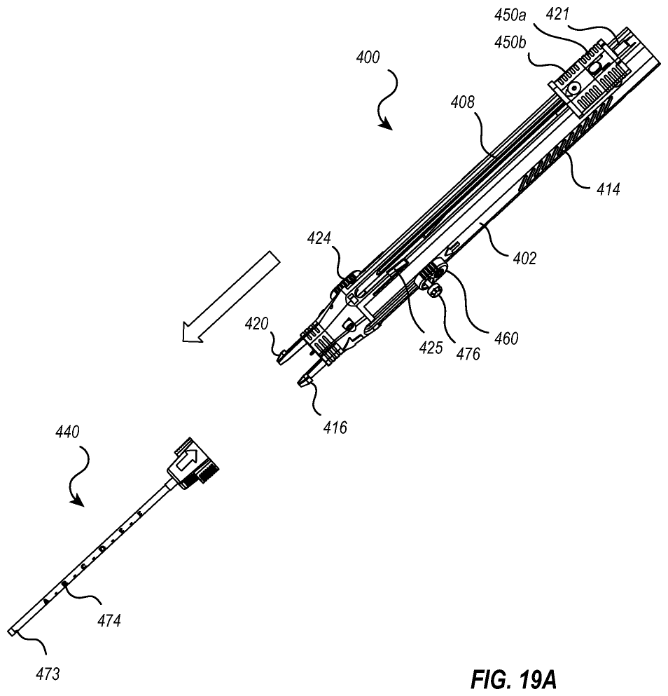

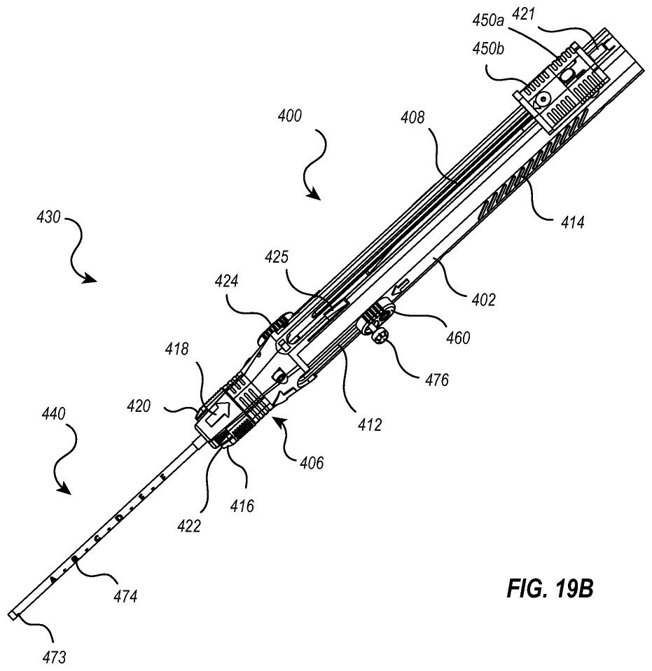

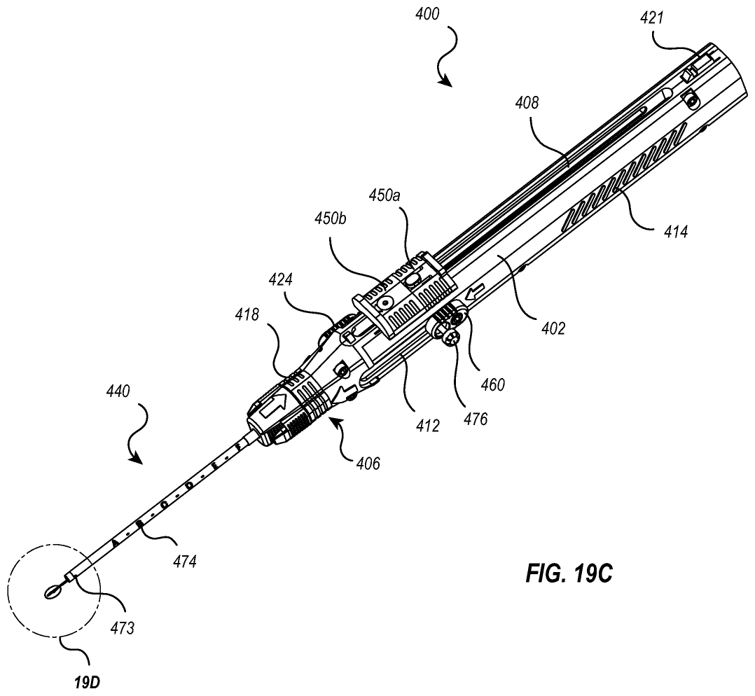

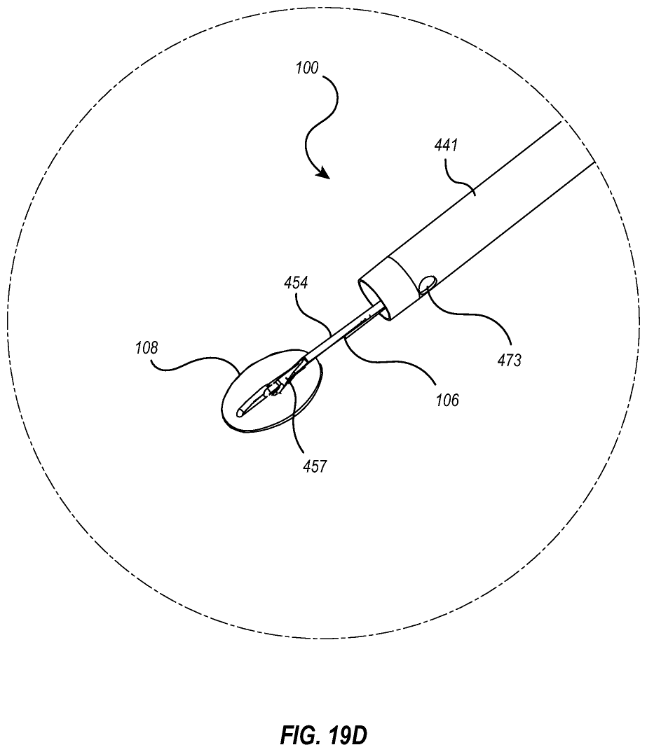

As shown in FIGS. 19A and 19B, the handle assembly 400 can be selectively attached to the delivery sheath 440 by inserting locking members 420 of the handle assembly 400 into the receiving members 468 of the delivery sheath 440 to form the delivery system 430. The locking members 420 can be made of a resilient material, such as flexible plastic, to allow the locking members 420 to flex when inserted into the receiving members 468. The locking members 420 can be flexed to disengage the hooked ends 422 to decouple the handle assembly 400 from the delivery sheath 440. As the locking members 420 cooperate with the receiving members 468, the chamber nozzle 429 penetrates the valve 462 to provide access to the sheath 441 for delivery and deployment of the closure device 100. When the delivery system 430 is engaged to deploy the closure device 100, as in FIG. 19C, the slider 450 can be moved in a distal direction towards the delivery sheath 440, which can cause the anchor 108 of the closure device 100 to be deployed.

FIG. 19D illustrates a close-up view of the partially-deployed closure device of FIG. 19C. The forked end 457 of the push wire 454 deploys the anchor 108 of the closure device 100 out from the delivery sheath 441.

Method of Closure Device Insertion with Handle Assembly

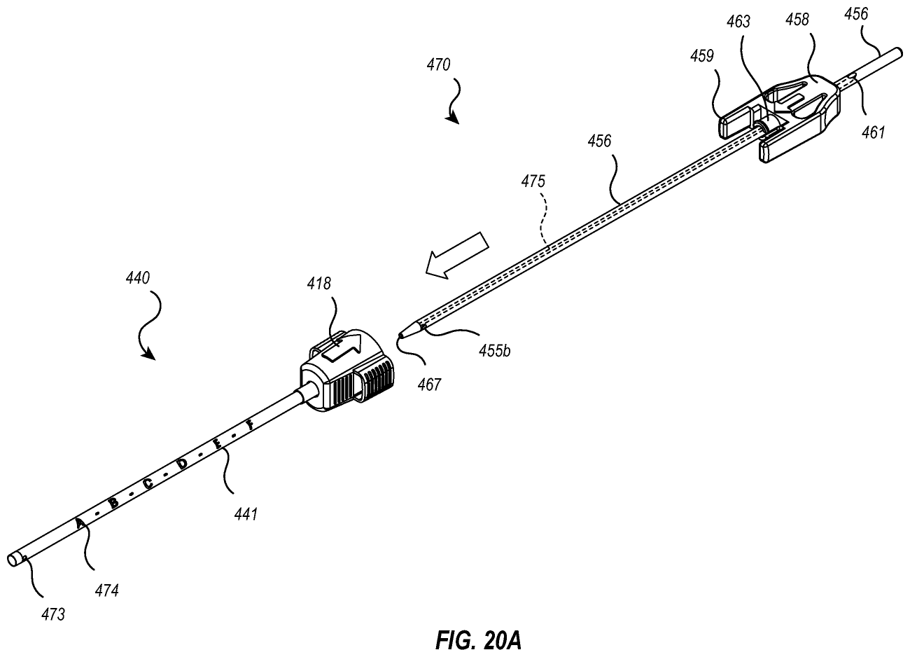

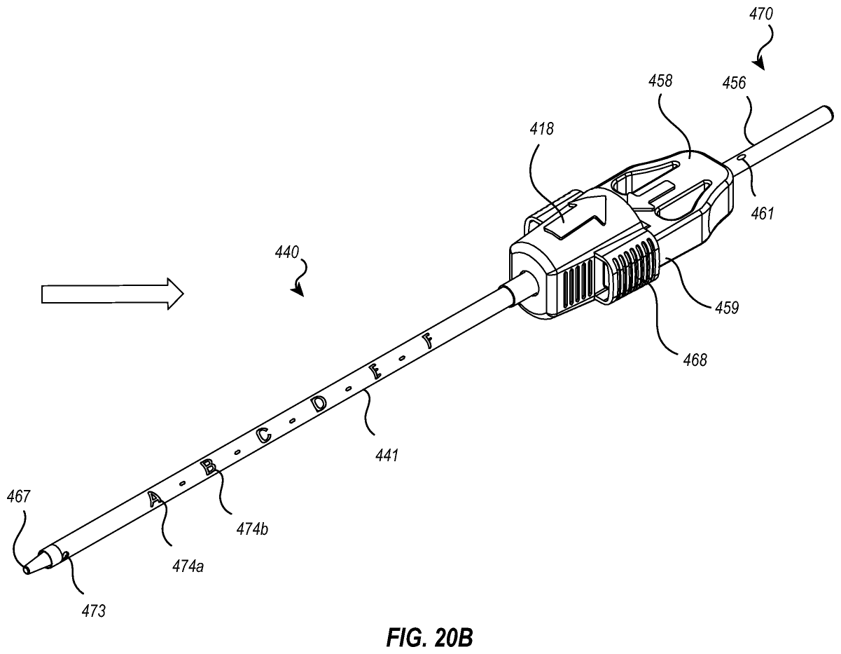

FIGS. 20A through 23C illustrate an example of a method of inserting a closure device using deployment system 430. First, the dilator tube 456 can be inserted into the delivery sheath 440. The dilator tube 456 can be selectively attached to the sheath 440 by connecting the dilator hub 458 to the sheath hub 418 in order to maintain the position of the dilator tube 456 in the delivery sheath 440 (FIGS. 20A-20B). The dilator tube 456 can be used to stretch the opening of the skin 16 and access tract 22 to allow for placement of a closure device 100.

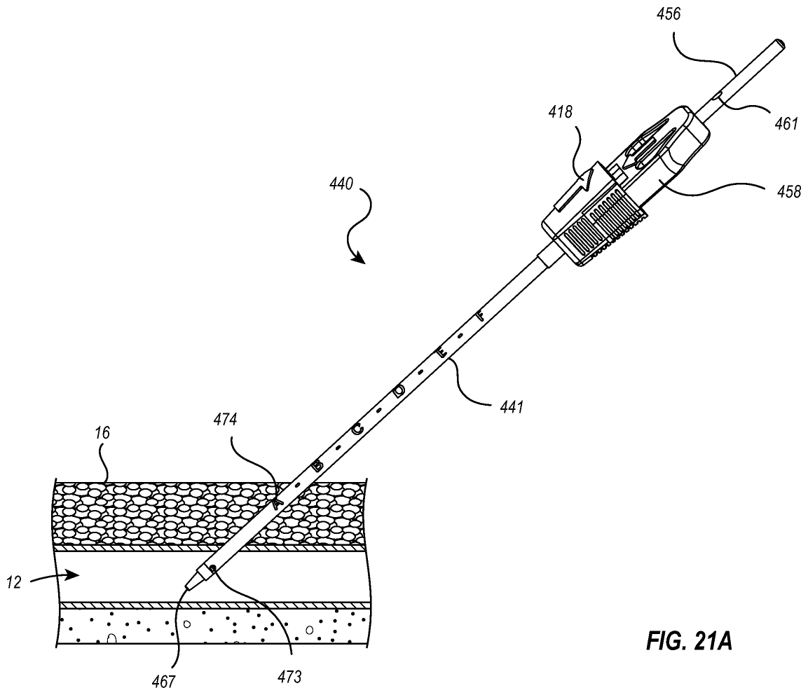

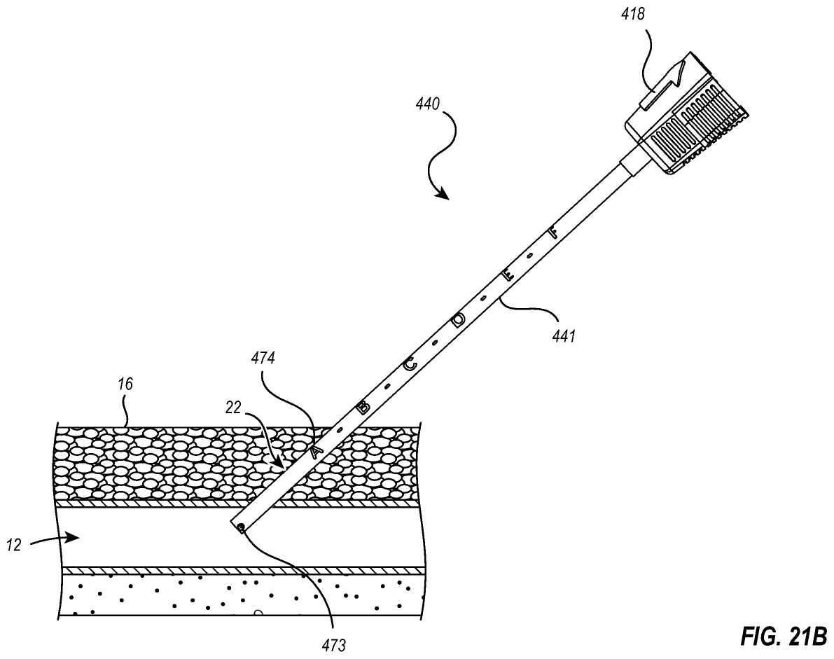

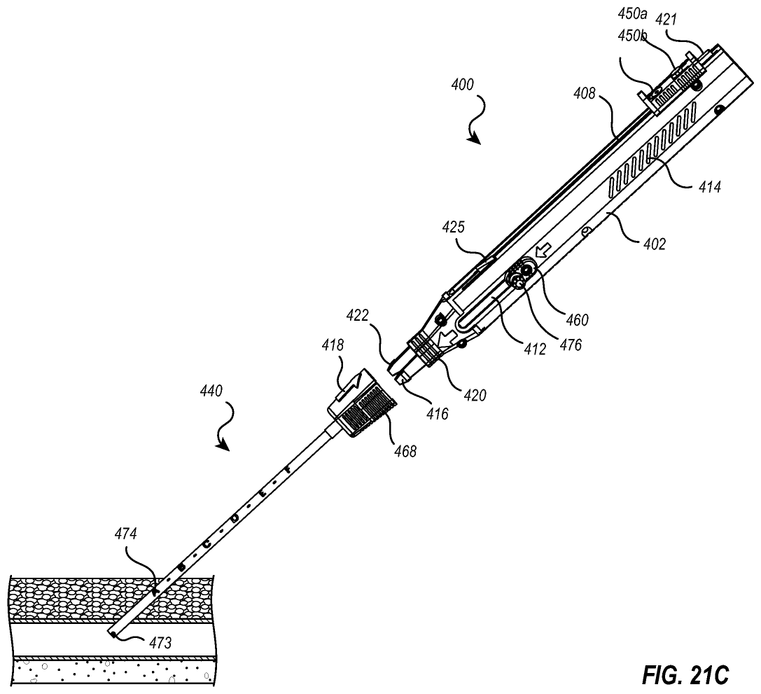

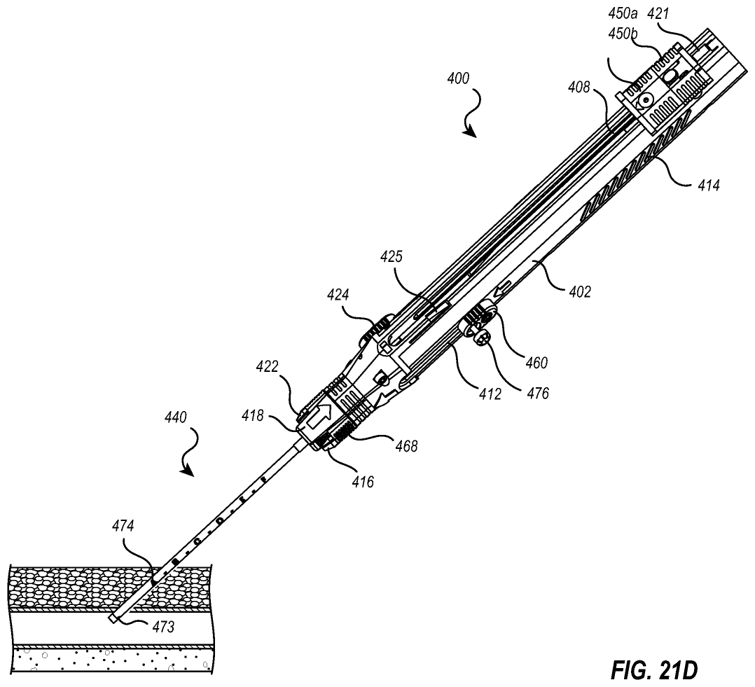

Next, the dilator hub 458 can be disengaged from the sheath hub 418 and the dilator tube 456 can be removed, as shown in FIG. 21B. The delivery sheath 440 can remain in the access tract 22. FIGS. 21C and 21D illustrate a method of connecting the handle assembly 400 to the delivery sheath 440. The handle assembly 400 can be selectively connected to the delivery sheath 440 by engaging the connecting members of the handle assembly 400 with the receiving members 468 of the sheath hub 418 of the delivery sheath 440.

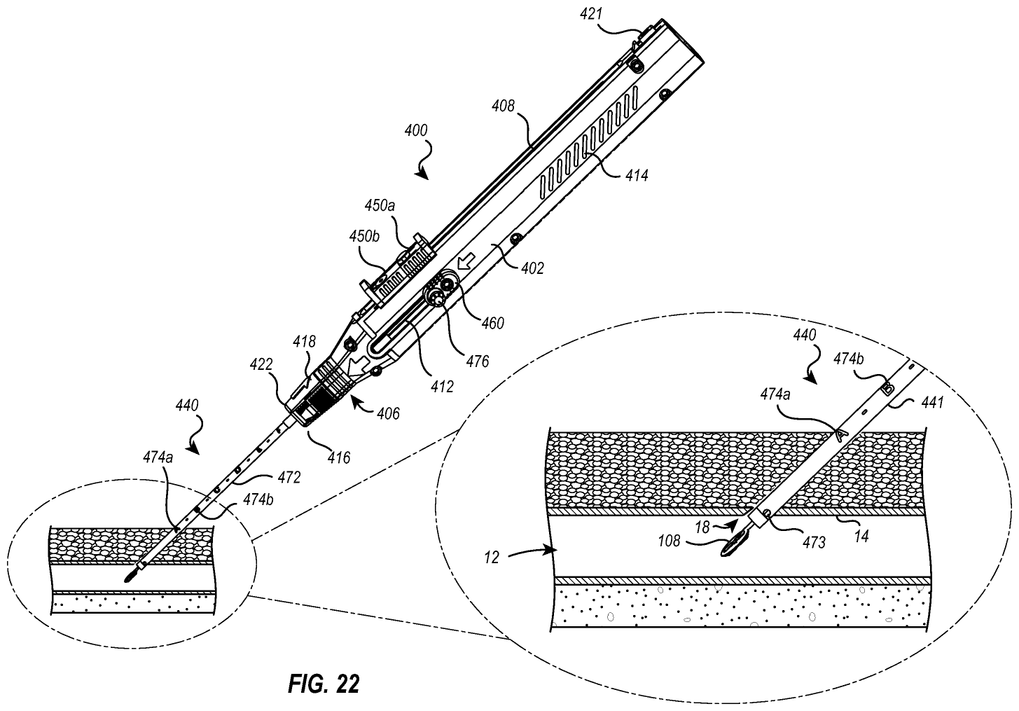

Once the handle assembly 400 is connected to the delivery sheath 440, the practitioner can depress the proximal locking assembly 421 to unlock the slider 450 and push the slider 450 in a distal direction towards the distal end 406 of the handle body 400, as illustrated in FIG. 22. This causes the delivery system 430 to eject the anchor 108 into the blood vessel lumen 12 so that the anchor 108 can contact the vessel wall 14 and be positioned on the puncture or access site 18. Once the slider 450 reaches the distal end 406, the anchor 108 should be ejected (fully) from the delivery system 430, with the cap 102 and fluid-blocking component 104 remaining in the tamper tube 442 of the implant assembly 426 within the delivery sheath 440. As the support tube 454 and the implant assembly 426 are contained within the delivery sheath 440, they are not illustrated in FIG. 22.

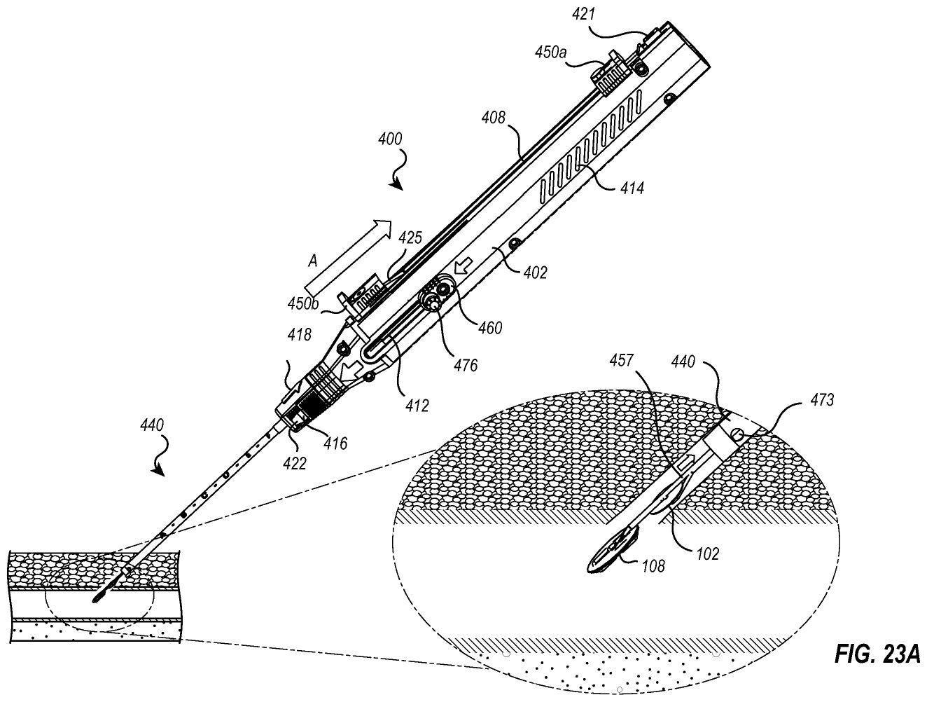

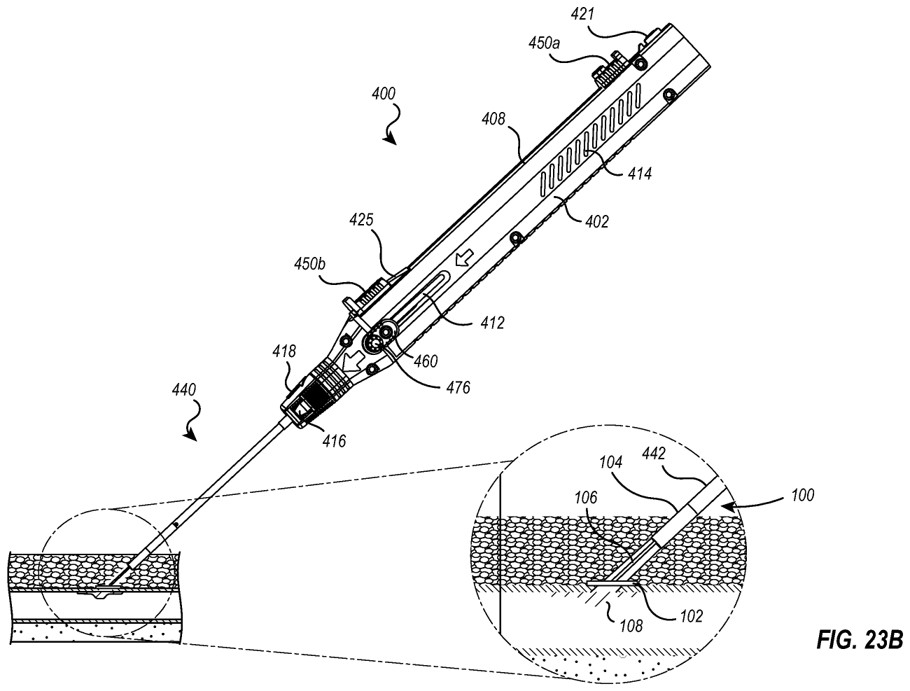

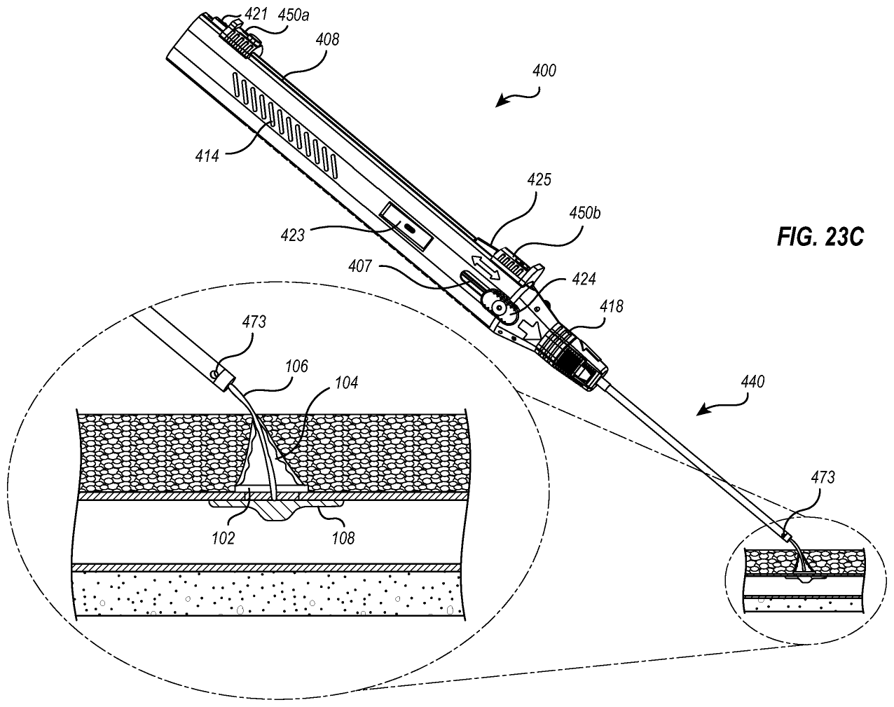

Turning to FIG. 23A, the slider 450 can be configured to slide along the elongate opening 408 until portion 450b slides past locking assembly 425, at which point locking assembly 425 can lock slider portion 450b at the distal end of elongate opening 408. The locking assembly 425 can be formed with the handle body 402, such as having a living hinge connection with the handle body 402 or can be a separated mechanism connected or mounted to the handle body 402. Once portion 450b is locked by the locking assembly 425, the practitioner can depress portion 450a to release interlocking end 466a from interlocking end 466b, effectively releasing portion 450a from portion 450b. Portion 450a, to which the push wire bend 477 of the push wire 452 is mounted, can be moved proximally to retract the push wire 452 in a proximal direction from the tissue and into the handle assembly 400. Retracting the push wire 452 can cause the anchor 108 to rotate and become substantially parallel with the blood vessel wall.

After the anchor 108 is deployed, the practitioner gently pulls the handle assembly 400 in a proximal direction, causing anchor 108 to engage and seat against the inner surface of the vessel wall by virtue of a tension force applied by the suture 106 connecting anchor 108 to handle assembly 400. While maintaining this force, a practitioner can engage the cap slide 460 by depressing plunger 476 and pushing the cap slide 460 in a distal direction toward the distal end 406 of the handle assembly 400. FIG. 23B illustrates the cap slide 460 engaging the tamper tube 442 (or a portion of the carriage 438) to eject the cap 102 from the delivery system 430 and press cap 102 against the outer surface of the vessel wall. However, handle assembly 400 also includes a lock-out/interlock assembly, the details of which will be described below, that prevents cap slide 460 from being prematurely actuated, which, in turn, prevents the cap 102 and sealant 104 from being prematurely deployed. As discussed in greater detail below, this lock-out mechanism prevents cap slide 460 from being moved from its initial position until the anchor 108 has been deployed and properly placed within the vessel and a predetermined amount of tension is applied to the suture 106 by the practitioner.

After the anchor 108 and cap 102 are placed, the sealant 104 is then deployed. This is done by moving the cap slide 460 in a proximal direction, which causes tamper tube 442 to also move in a proximal direction, while maintaining support tube 454 stationary. This proximal movement of the cap slide 460 and stationary position of the support tube 454 combination exposes the sealant 104 to blood and tissue within the tissue tract 22. Once the sealant 104 has been positioned against the proximal side of cap 102 within tissue tract 22 in this manner, then the release button 424 can be engaged to release the suture 106 and closure device 100 from the delivery system 430, with the sealant 104 remaining in the tissue tract 22, as illustrated in FIG. 23C. Thereafter the suture 106 can be trimmed at or below the level of the skin or tissue.

### Locking Assemblies of the Vessel Closure Delivery System

Also disclosed are lock-out or latch assemblies that are incorporated into delivery system 430. The disclosed lock-out assemblies are configured to, among other things, (i) provide the practitioner with audible, visible, and/or tactile feedback during operation of the device and placement of the closure device; (ii) prevent the practitioner from prematurely deploying a component of the implant and/or prematurely removing the delivery device from the patient; (iii) ensure the elements of the closure device are properly positioned and in the correct order; (iv) improve overall patient safety; and/or (v) improve the speed, simplicity, and efficiency of placement of the closure device.

For example, a first interlock assembly is provided at the proximal end of handle 402, which prevents slider 450 from being accidentally or prematurely moved from its initial position. Once delivery system 430 has been properly coupled to delivery sheath 440, and the practitioner is ready to introduce the closure implant 100 through the delivery sheath 440 and into the vessel, the practitioner must first depress proximal lock assembly 421, which unlocks the slider 450 and allows the slider 450 to be moved from the proximal end to the distal end of the handle assembly 400, which, in turn causes the internal components of the delivery system 430 to be advanced in a distal direction through the delivery sheath 440. While a particularly lock assembly 421 is shown and described, it will be appreciated that any of various other configurations could be provided, with similar functionality.

A second interlock assembly is configured to interact with a carriage assembly 438 housed within the handle assembly 400. The carriage assembly 438 is configured to deploy the closure device 100 and thereby provide substantially immediate hemostasis at a puncture site within a blood vessel wall. The interlock assembly prevents pre-mature deployment of one or more components of the closure device 100. For example, the interlock assembly prevents complete deployment of a cap seal until an anchor seal (deployed within the blood vessel) has been properly positioned against an interior of the blood vessel wall. Upon proper placement of the anchor and the application of a predetermined tensioning force, the interlocking assembly will release and allow the cap seal to be tightened against an exterior of the blood vessel wall.

Application of the pre-defined tensioning force simultaneously causes, at least, (i) the cap seal to be properly angled against an outer surface of the blood vessel wall; (ii) a spring mechanism to retract and clear a deployment path for a sealant; (iii) pivot a latch to unlock a component of the carriage assembly that tightens the cap against the blood vessel wall; and/or (iv) proper placement of the anchor against an interior surface of the blood vessel wall. The practitioner can then engage the carriage assembly to tighten the cap against the blood vessel wall and deploy the sealant.

The interlock assembly can also prevent the sealant from being retracted into the handle assembly as various elements of the handle assembly are retracted during placement of the closure device and removal of the handle assembly from the closure device. The sealant is positioned against the cap seal and must remain in place against the cap seal. Once the cap seal has been positioned against the puncture site, the interlock assembly unlocks a component of the carriage assembly, allowing the carriage to move proximally. This proximal movement unsheathes the sealant while ensuring the sealant stays in position against the cap seal and within a tissue tract leading to the puncture site. Only by staying in place against the cap seal and within the tissue tract can the sealant react with bodily fluids to expand and seal the tissue tract.

Once the sealant has been unsheathed, components of the interlock assembly move proximally along the handle assembly. The proximal movement of the interlock assembly components releases or untethers the suture from the handle assembly, which allows the handle and dilator assemblies to be removed from the patient while leaving the anchor, cap, sealant, and suture in place. During the process of detaching the closure device from the delivery device, the delivery device may produce a “snap” or “click” inside the handle, which the practitioner can both hear and feel, indicating the closure device has been completely disengaged from the handle assembly. Without the interlock assembly, a practitioner may attempt to prematurely remove the handle and dilator assemblies, potentially ripping the closure device from the patient and causing further tissue damage.

To assist the practitioner in properly deploying the closure device, disclosed handle assemblies can include a variety of visual markings and indicators. For example, a practitioner initially inserts a dilator assembly to provide access to the puncture site. Depth indicators on the dilator assembly allow the practitioner to visibly determine how far into the blood vessel and/or the tissue tract the dilator assembly has been advanced. This allows the practitioner to know if they have reached the puncture site or not. Once the practitioner has accessed the puncture site and deployed the anchor within the blood vessel, markings on the distal end of the handle assembly visually communicate to the practitioner when the cap has been positioned against the puncture. For example, the markings can be in the form of bars indicating 5, 4, 3, and 2 mm of distance from the puncture site. It should be understood that the markings can be more or less granular, as needed.

These markings help a practitioner orient how far from the vessel they are. Additionally, and/or alternatively, the markings help orient the practitioner to how deep within a tissue tract they are. Specifically, if the practitioner is feeling tactile or other resistance while deploying the closure device, the physician can check the markings and “see” how far from the puncture site they actually are. Once the practitioner is within range of the puncture site and deploying the closure device, components of the interlock assembly begin interacting with components of the handle and slider/carriage assemblies to ensure proper deployment of the components of the closure device. The markings can be included on both sides of a handle assembly, allowing practitioners of both handedness to use the disclosed devices.

Other components within the handle assembly may interface with components of the interlock assembly to provide audible feedback to the practitioner, in addition to visual and tactile feedback. For example, a slider/carriage assembly that is configured to deploy the closure device can include grooves or protrusions to mechanically interface with the interlock assembly components. When the interlock assembly components unlock components of the slider assembly, the physician will audibly hear the interlock assembly components hit the grooves or protrusions of the slider assembly.

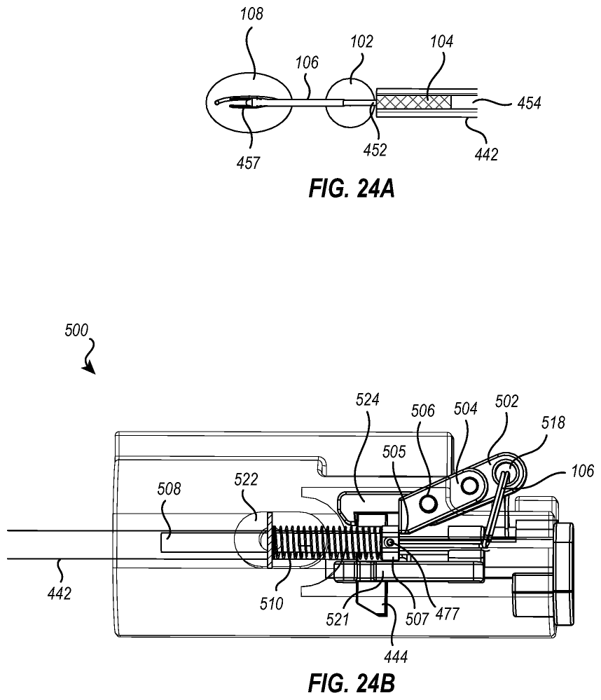

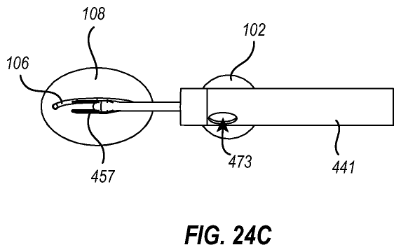

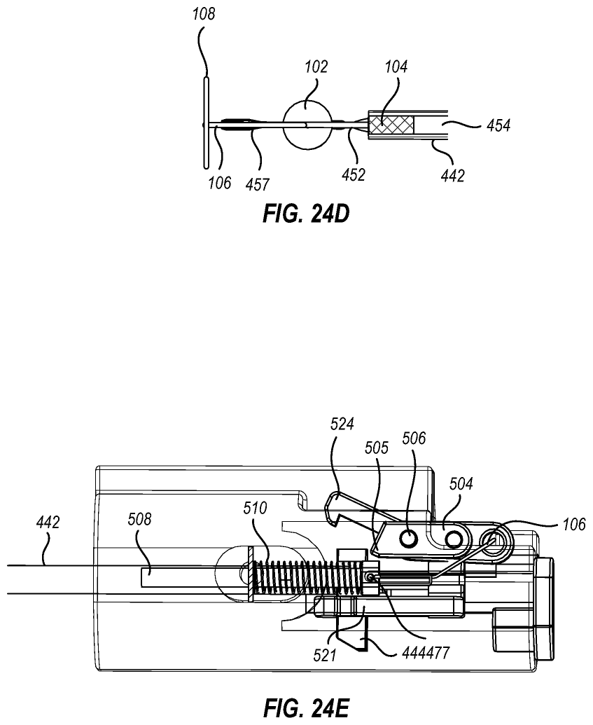

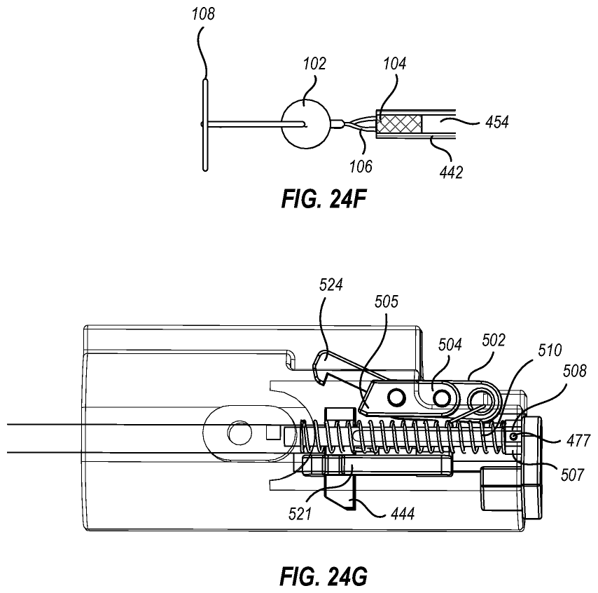

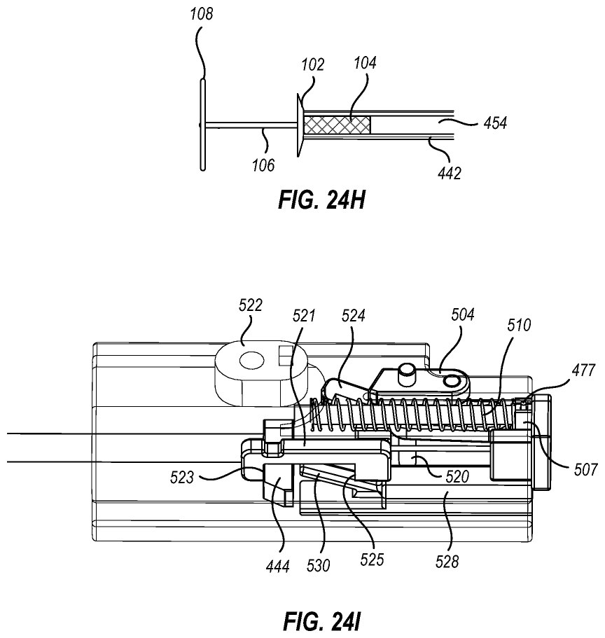

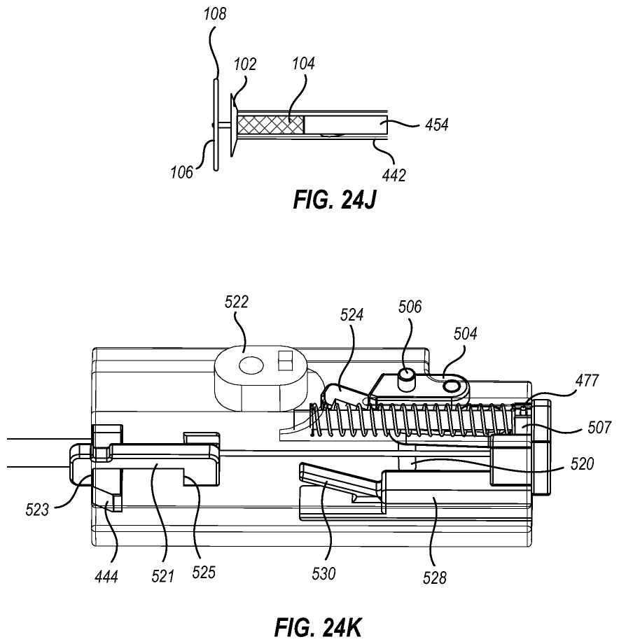

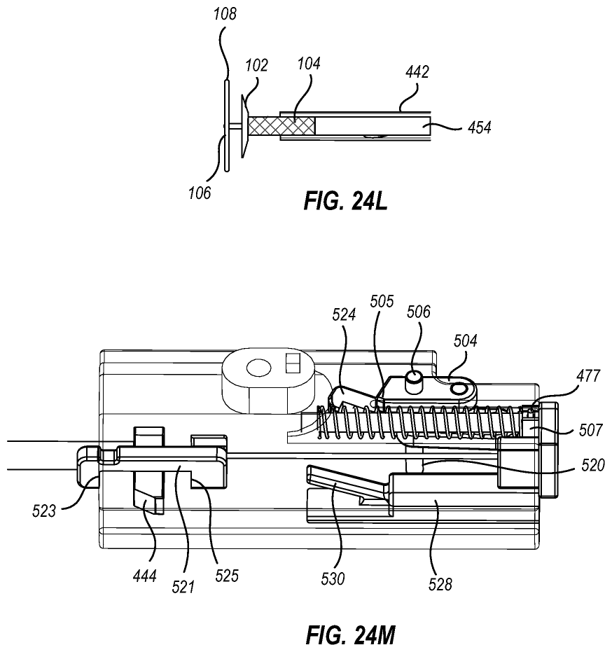

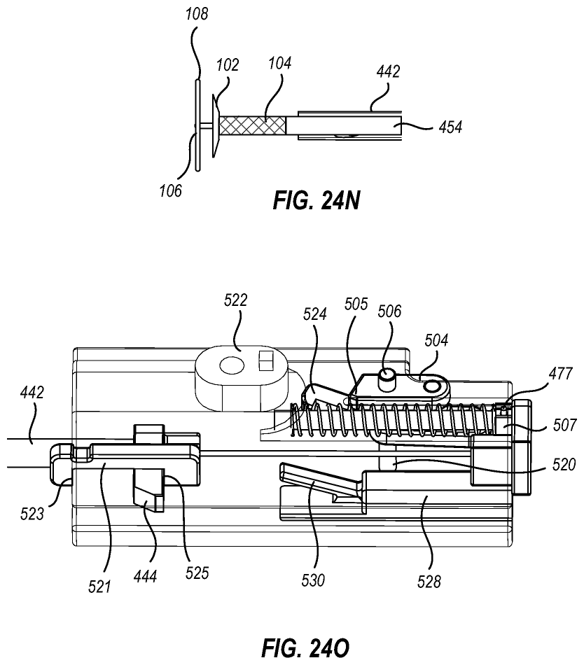

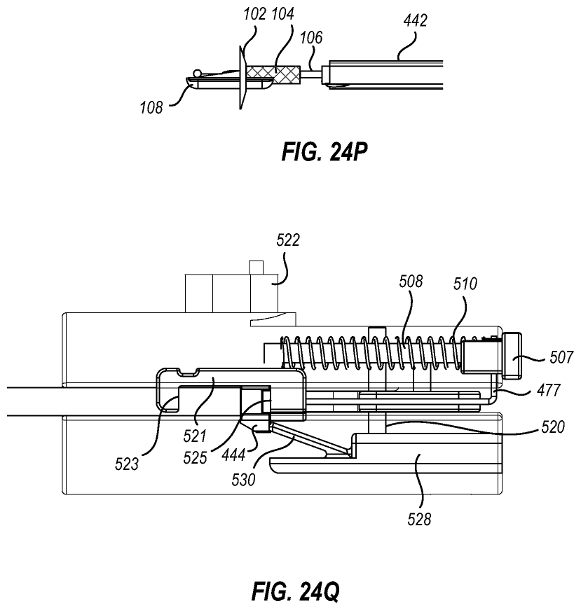

FIGS. 24A-24Q illustrate an interlock or latch assembly 500 that can be used in conjunction with carriage 438 (such as carriage 438 illustrated in FIGS. 14C-14F and 16) to release the suture 106 from the handle assembly 400, as an alternative to the pin 417 and bore 415 mechanism shown in FIGS. 14D-14F. FIGS. 24A-24Q also illustrate the corresponding position of the closure device 100 during movements and operation of the latch assembly 500. The interlock or latch assembly 500 is configured to perform the functions of the interlock assembly described herein. Specifically, the closure device 100 (including the anchor 108, cap 102, sealant 104, and suture 106) is illustrated at various stages of deployment.

FIG. 24A illustrates the anchor 108 that has been advanced into a blood vessel via the forked end 457 of the push wire 452. The length of push wire 452 is sized such that, when slider 450 is moved to its distal-most position on handle assembly 400, the anchor 108 is advanced just beyond the distal end of the delivery sheath 440 (as illustrated in FIG. 24C), and into the lumen 12 of the blood vessel 10. At that point, the cap 102 is still located outside the blood vessel and contained within delivery sheath 440, and the sealant 104 is still contained within the tamper tube 442. FIG. 24C illustrates the delivery sheath 441 at the moment after deployment of the anchor 108 within the blood vessel 10. At this point, the practitioner can pull back on the handle assembly 400 in a proximal direction. The suture 106 is connected to the anchor 108 at its distal end and connected to the handle assembly 400 proximate its proximal end. As the physician pulls the handle assembly 400 in a proximal direction, the suture 106 will apply a tension force to the anchor 108. The tension force applied to the anchor 108 causes anchor 108 to move in a proximal direction and seat against the inner wall of the vessel 10 and over the puncture site 18. In addition, the overall length of the suture 106 relative to the overall length of the push wire 452 is selected such that, during initial proximal movement of the handle assembly 400, the forked end 457 of the push wire 452 disengages from the anchor 108 before the slack is taken out of the suture 106. This allows anchor 108 to unfurl and reorient itself from its initial collapsed, pre-deployment configuration/orientation to its fully deployed configuration/orientation. The anchor 108 reorients before the suture 106 applies a tensioning force to anchor 108 and draws it up against the inner surface of the blood vessel wall.

As illustrated in FIG. 24B, the latch assembly 500 can comprise internal components including a first latch or lock lever 502 and a second latch or toggle 504. The first latch 502 and second latch 504 can be maintained by a latch or connecting pin 506 positioned at the distal end of the handle body 402 and proximal to the chamber assembly 427. First and second latches 502, 504 are both rigidly coupled to connecting pin 506, and connecting pin 506 is rotatably coupled to an internal portion of carriage 438. Thus, when a transverse force is applied to the proximal end of the first latch 502, such force causes the first latch 502, the second latch 504, and the connecting pin 506 to all rotate together about a pivot point formed by the connecting pin 506. The second latch or toggle 504 can interact or engage with the push wire 452. The latch assembly 500 can be configured to interface with the carriage 438 and interface with elements contained within and/or on the handle body 402. In some embodiments, the latch assembly 500 is housed within the internal slider 438.

The first latch 502 can include at least one or more openings 518 through which the suture 106 can be threaded and looped around a pin 520. The pin 520 can be in mechanical communication with a slider 522, which can be situated within a track 524 on the handle body 402. The slider 522 can also be in mechanical communication with the carriage 438 and support tube 454. When moved in a distal direction, the slider 522 can actuate the support tube 454 to tamp the cap 102 of the closure device 100. When moved in a proximal direction, the slider 522 can cause the pin 520 to be moved in a proximal direction, thereby releasing the suture 106 from the delivery system 430. Proximal movement of the slider 522 can also retract the support tube 454 to expose and place the sealant 104.

The second latch 504 can include a hook 524, configured to receive the push wire bend 477 and selectively lock the push wire 452 in a deployed position. The stopper 444 is in mechanical communication with the support tube 454, such that proximal movement of the stopper 444 causes actuation of the support tube 454 to deploy the cap 102. The hook 524 may additionally receive the stopper 444, locking the stopper 444 in place and preventing deployment of the cap 102. When tension is applied to the suture 106, the tensioned suture 106 can cause the first latch 502 to open, subsequently releasing or opening the second latch 504 to allow movement of the push wire 452. When the suture tension reaches a threshold minimum (e.g., of approximately 0.8 pounds of force (lbf)), the latch assembly 500 is tripped, causing the push wire 452 to retract (e.g., approximately 15 mm) and the slider 522 to unlock. The suture tension may range from approximately 0.8 lbf+/−0.3 lbf, such as 0.5 lbf to 1.1 lbf. Such a suture tension is optimized for the gentle delivery and placement of the anchor 108 against the inner wall of the blood vessel and within the acceptable range of force applied by practitioners trained in percutaneous catheter procedures.

As alluded to above, FIG. 24B illustrates the initial, locked position of first and second latches 502 and 504. In this position, the relative positions of the push wire 452, internal slider 522 and cap slide 460 are locked or fixed in place. This lockout mechanism prevents the practitioner from prematurely or inadvertently deploying cap 102 and sealant 104 before it is time to do so. Due to the lockout mechanism, the cap 102 and sealant 104 can only be deployed after anchor 108 has been properly positioned within the vessel and over the puncture site and tension is applied to and maintained via suture 106.

In the initial, locked position, the hook 524 of first latch 502 is engaged over and against stopper 444 (as illustrated in FIG. 24B). This engagement by hook 524 prevents slider 522 and cap slide 460 (see FIG. 23A) from being moved in a distal direction. Once a predetermined amount of tension is applied to the suture 106, the tension causes that portion of the suture 106 that is looped though hole 518 to exert a force on the proximal end of first latch 502, which, in turn, causes both latches 502, 504 to rotate about the pivot point of connecting pin 506 (latches 502 and 504 both being rigidly coupled to connecting pin 506). This rotation causes hook 524 to rotate away and disengage from stopper 444, thereby enabling slider 522 to be moved in a distal direction by the practitioner via the cap slide 460 (see FIG. 24B). As discussed elsewhere, distal movement of slider 522 causes tamper tube 442 and support tube 454 to move in a distal direction relative to the delivery sheath 440 to deploy the cap 102 and/or press the cap 102 up against the outer surface of the vessel wall.

In addition, in the initial, locked position, a distal end 505 of the second latch 504 engages collar 507. Collar 507 is mechanically coupled to push wire bend 477, in that push wire bend 477 extends transversely though a hole 509 in collar 507, as illustrated in FIG. 24B. As also shown in FIG. 24B, in the initial locked position, spring 510 is held in compression between a distal shoulder of collar 507 and an internal surface formed in carriage 438. As discussed, once a sufficient tension force is applied to the suture 106, the second latch 504 rotates about the connecting pin 506, freeing collar 507 to move in a proximal direction under the force of spring 510 as it expands as a result of its spring force. This proximal movement of collar 507 also causes the push wire 452 to retract in a proximal direction and into chamber assembly 427 (see FIGS. 14A and 14B). When released, the spring force of spring 510 causes the collar 507 to be rapidly propelled in a proximal direction until it collides with a proximal wall surface of carriage 438. The collision produces audible and tactile feedback to the practitioner indicating that: the anchor 108 has been deployed and positioned; that the locking mechanism associated with the cap slide 460 has been released or disengaged; and that the practitioner may proceed with the next step of deploying the implant 100.

FIG. 24D illustrates the initial few millimeters of the push wire's 452 retraction. As illustrated in FIG. 24E, the hook 524 pivots in response to the applied suture tension. Otherwise, the hook 524 always stays in a locked position, preventing movement of the slider 522. The hook 524 only unlocks or pivots in response to adequately applied suture tension. In addition, tension applied to the suture 106 also causes the second latch 504 to rotate away and disengage from the collar 507, freeing the collar 507 and push wire 452 to move in a proximal direction.

In FIG. 24F, the push wire 452 has been fully retracted and is no longer visible. The anchor 108, a portion of the suture 106, and the cap 102 are clearly visible.

The latch assembly 500 can also include a rod or spring guide 508 extending in a longitudinal direction and connected to the carriage 438 via the push wire bend 477. The proximal end of the rod 508 can include the collar 507 with a bore 509 configured to receive the push wire bend 477. The rod 508 can have a spring 510 coiled around it, which spring 510 has a proximal end and a distal end. The proximal end of the spring 510 can be fixed against the collar 507. The distal end of the spring 510 can remain unattached from the rod 508 to allow the spring 510 to be compressed between a distal interior shoulder formed in the carriage 438 and collar 507. As illustrated in FIG. 24G, in response to the applied suture tension, the spring 510 decompresses and causes the collar 507 and rod 508 to retract in a proximal direction, pulling the push wire 452 with them. This causes the distal end of push wire 452 to retract back into chamber 427 (see FIGS. 14A and 14B). Retracting the push wire 452 also reduces possible interference with deployment of the sealant 104.

With the interlock mechanism of latch assembly 500 disengaged and unlocked, the practitioner may proceed with the next step of deploying implant 100, namely, advancing and tamping the cap 102 against the outer surface of the vessel wall. This can be done by the practitioner moving the cap slide 460 in a distal direction. The cap slide 460 is mechanically coupled to slider 522, slider 522 is mechanically coupled to stopper 444, and stopper 444 is mechanically coupled to tamper tube 442. Thus, the cap slide 460, slider 522, stopper 444, and tamper tube 442 move together as a single unit. Thus, when the stopper 444 is engaged by the first latch 502, none of the foregoing elements can be moved in a distal direction. However, once unlocked, movement of the cap slide 460 in a distal direction by the practitioner will cause the other coupled components to move also. The distance of travel of the tamper tube 454 is constrained by the travel distance of the cap slide 460 and the internal dimensions of the carriage 438 (as schematically illustrated in FIGS. 24I and 24K). In any event, as illustrated in FIGS. 24H-24K, as the stopper 444 is advanced in a distal direction, the tamper tube 442 engages a proximal surface of cap 102 and pushes the cap 102 in a distal direction until it comes into intimate contact with the outer surface of the vessel wall.

The latch assembly 500 can also include a first slider block 521 situated inside of the carriage 438 and can include pin 520. First slider block 521 is mechanically coupled to a proximal end of the support tube 454. First slider block 521 has a distal shoulder 523 and a proximal shoulder 525, which both interact with stopper 444. As the stopper 444 is moved in a distal direction, a distal surface of stopper 444 engages distal shoulder 523 of first slider block 521, whereby further distal movement of stopper 444 also causes first slider block 521 (and, thus, support tube 454) to also move in a distal direction. Thus, when the latching mechanism of latch assembly 500 is disengaged and unlocked, and the practitioner moves the cap slide 460 in a distal direction, all of the following components move together in a distal direction as a single unit: cap slide 460, slider 522, stopper 444, tamper tube 442, first slider block 521, and support tube 454. Once these components reach the end of travel in a distal direction, and the cap 102 is firmly positioned against the outer surface of the vessel wall, then the practitioner can begin moving the cap slide 460 in a proximal direction.

This proximal movement causes the stopper 444 and tamper tube 442 to also move in a proximal direction. However, until the stopper 444 moves proximally a sufficient distance such that stopper 444 engages the proximal shoulder 525 of the first slider block 521, the support tube 454 remains in its distal-most position. The distance between the distal shoulder 523 and proximal shoulder 525 of the first slider block 521 is approximately the same as the length of sealant 104 (e.g., approximately 5-8 mm). Thus, this proximal movement of the tamper tube 442 relative to the support tube 454 exposes substantially the entire length of the sealant 104 to adjacent tissue and blood within the surrounding tissue tract. The stationary positioning of the support tube 454 while the tamper tube 442 is retracted helps ensure that the sealant 104 remains pressed up against the proximal surface of cap 102 while it absorbs surrounding bodily fluids and expands within the tissue tract. Once the stopper 444 engages the proximal shoulder 525 of the first slider block 521, further proximal movement of the cap slide 460 causes the tamper tube 442 and support tube 454 to retract together until the cap slide 460 reaches the proximal end of its travel.

In some embodiments, the first slider block 521 can house the pin 520 which can hold the suture loop 526. The first slider block 521 can also have a window or channel through which the suture loop 526 can be released when release is initiated by the slider 522. Additionally, the latch assembly 500 can also include a pin body 528, which can include a wedge 530 having an angled face and a vertical face. The angled face can help to maintain the stopper 444 in the locked position when it is latched/received by the first latch 502. Once the first latch 502 is released by tensioning the suture 106, the stopper 444 is released. The slider 522 can then be moved in a distal direction to tamp the cap 102 and then moved in a proximal direction to release the suture 106 from the bolt/pin 520. For example, FIG. 24H illustrates the cap 102 and the tamper tube 442 being configured to move the cap 102 distally towards the anchor 108. Distal movement of the first slider block 521 will cause the cap 102 to be advanced distally. The sealant 104 is located adjacent to the cap 102 and is still housed within tamper tube 442.

FIGS. 24J and 24L illustrate deployment of the sealant 104 and retraction of the tamper tube 442 to expose the sealant 104 to blood and other bodily fluids. The support tube 454 stays in place (i.e., stationary), keeping the sealant 104 in place against the (now deployed) cap 102 during retraction of the tamper tube 442. FIG. 24N illustrates the sealant 104 being fully unsheathed and exposed. At this point, the tamper and support tubes 442, 454 can move proximally together, as one, via proximal movement of slider 522, stopper 444, and first slider block 521. In FIG. 24P, continued retraction of the tamper and support tubes 442, 454 via proximal movement of slider 522, stopper 444 and first slider block 521 releases the proximal end of the suture 106 from the carriage 438 (as described below), which thereafter permits complete separation of the delivery device from the closure device 100.

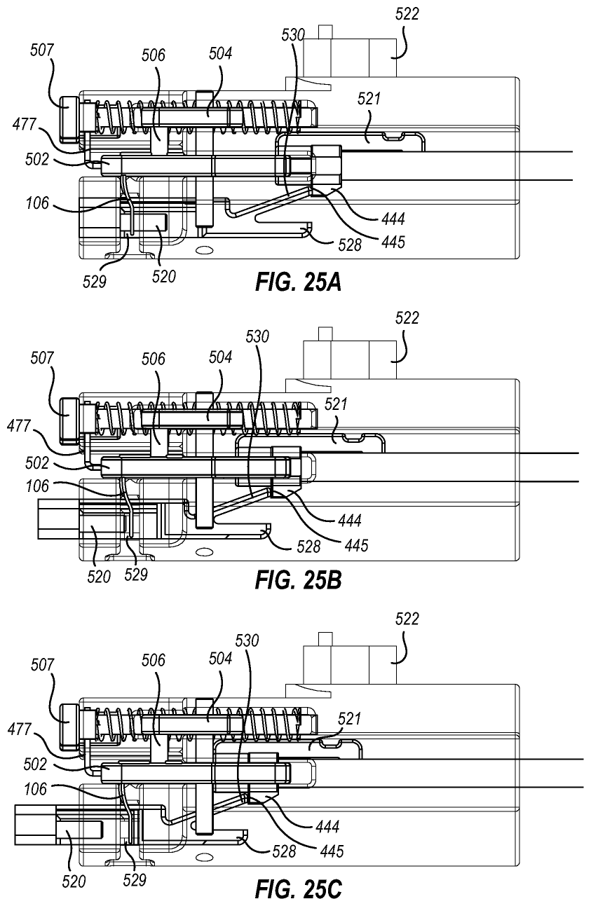

FIGS. 25A-25C illustrate a bottom view of the latch assembly 500 and illustrate release of the suture loop 526 from the pin 520. Initially, as illustrated in FIG. 25A, the proximal end of suture 106 terminates in a suture loop 526, which is looped around a pin 520 that extends through a recess 529 formed within the carriage 438. At this point, the suture loop 526 is constrained by the pin 520 and the pin 520 is mechanically coupled to a proximal end of a pin body 528. Located at a distal end of the pin body 528 is a resilient, flexible finger 530 that is configured to interact with stopper 444. FIG. 25A illustrates the initial position of the pin body 528 and its position up to the point that the cap 102 and sealant 104 have been deployed, and tamper and support tubes 442,454 are in the process of being retracted in a proximal direction. At a certain point in the proximal travel of the tamper and support tubes 442, 454, a proximal shoulder portion 445 of stopper 444 comes into contact with a distal end of finger 530 of the pin body 528. At that point, further proximal movement of the stopper 444 causes the pin body 528 to also move in a proximal direction. Such proximal movement of the stopper 444, which is actuated via the cap slide 460 via slider 522, also causes the pin 520 to move proximally, freeing the suture loop 526 and the suture 106 from the device. An audible click may be heard to indicate that the suture 106 has been released from the device and it is now safe to remove the device from the patient.

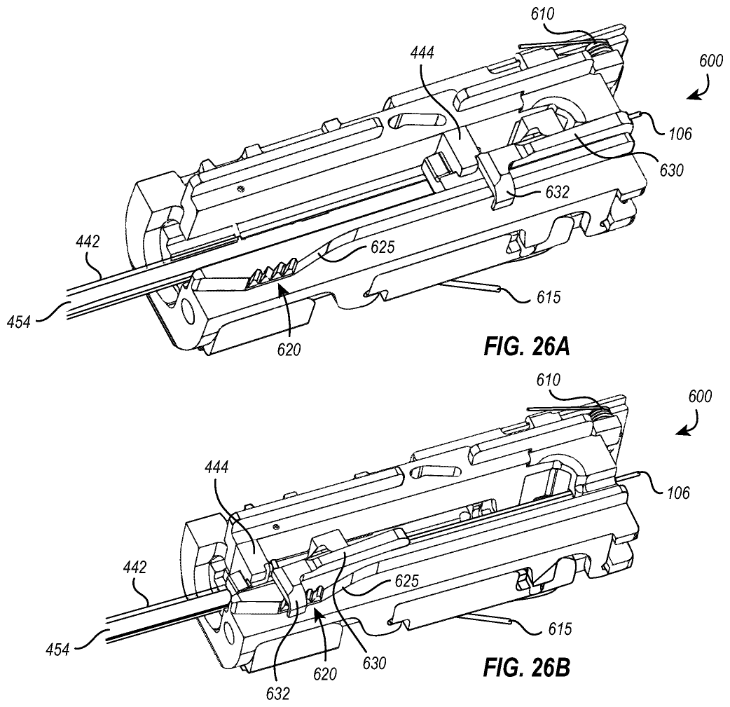

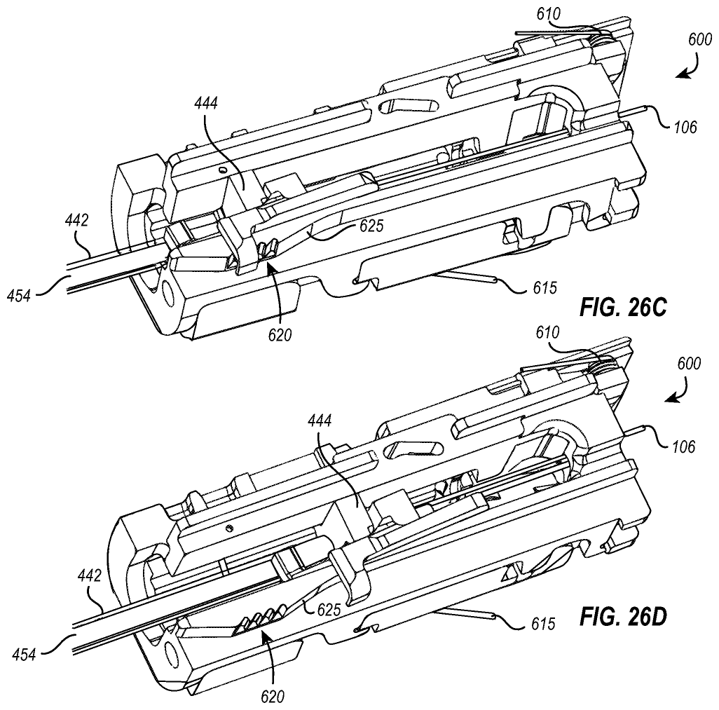

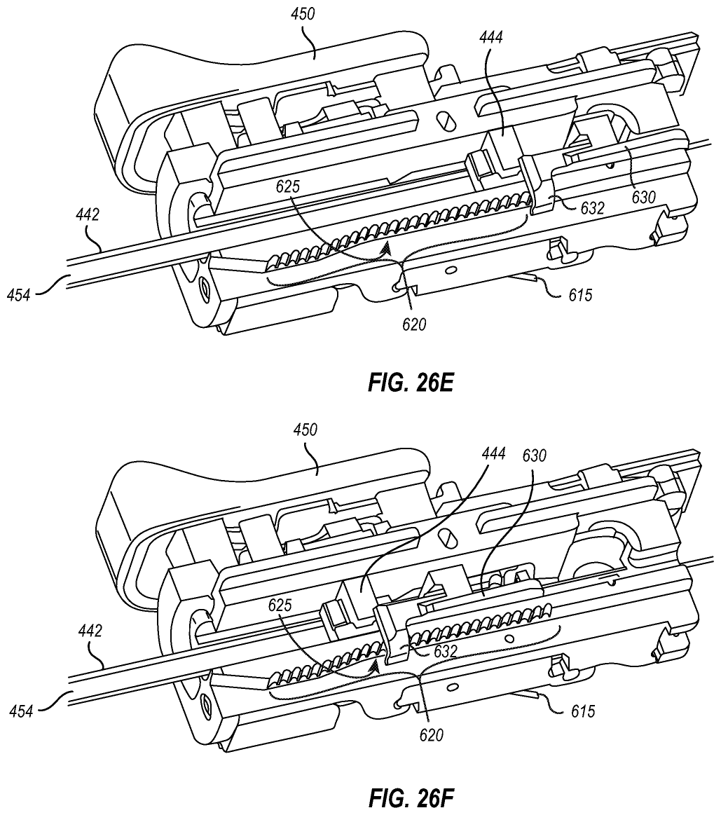

FIGS. 26A-26F illustrate another embodiment of a lock-out/latch assembly 600. The disclosure and discussion related to latch assembly 500 described herein is also applicable to the latch assembly 600 and so like structures are provided with like reference numerals. In some embodiments, the latch assembly 600 is contained within the carriage 438. The latch assembly 600 is configured to prevent proximal movement of the sealant 104 so that it cannot move away from the cap 102 when pulling back the tamper tube 442 to “unsheath” the sealant 104. For example, after the cap 102 has been pressed against the puncture site of the blood vessel, the practitioner moves the tamper tube 442 proximally to expose the sealant 104 to blood and other bodily fluids, causing the sealant 104 to expand. Latch assembly 600 is configured to ensure the sealant 104 stays positioned against the cap 102 and does not move back into the delivery sheath upon retraction of the tamper tube 442.

The latch assembly 600 can include a bearing surface 625 formed within the carriage 438, which bearing surface 625 includes a plurality of teeth 620. The number of teeth 620 may vary. For example, FIGS. 26A-26D illustrates an embodiment in which bearing surface 625 includes only four teeth 620 positioned proximate the distal end of bearing surface 625, whereas FIGS. 26E-26F illustrate an embodiment in which bearing surface 625 includes a larger number of teeth 620 positioned along a substantial portion of its surface. In either case, the teeth 620 can be angled toward the distal end of bearing surface 625 so as to provide a ratcheting action when the ratchet 630 (see below) is moved in a distal direction relative to bearing surface 625. In addition, the angle of the teeth 620 also prevents movement of the ratchet 630 in the opposite (proximal) direction whenever a complementary portion of the ratchet 630 is in engagement with any of teeth 620.

The ratchet 630 has a resilient, flexible arm 632 terminating in one or more teeth (not shown) that are complementary to, and configured to mate with, the teeth 620 of bearing surface 625. The ratchet 630 also engages and/or interfaces with the stopper 444 and the tamper and support tubes 442, 454. As described elsewhere, the support tube 454 can be coaxially positioned within the tamper tube 442 and the suture 106 can also be threaded therethrough.

When the stopper 444 is moved distally, the tamper tube 442 and the support tube 454 move together as one. During this distal movement, the stopper 444 engages the ratchet 630 and pushes the ratchet 630 in a distal direction along with it. As first slider block 630 moves distally, flexible arm 632 also moves distally and travels along bearing surface 625, bending laterally as it travels along the curved path of bearing surface 625. At a certain point in the distal travel, the teeth (not shown) of arm 632 will engage with the teeth 620 of the bearing surface 625. With further distal travel, the flexible and resilient nature of the arm 632 will allow its teeth to jump from tooth to tooth of the bearing surface 625 in a ratcheting manner, which, in turn, will provide both audible and tactile feedback to the practitioner. Once the teeth are engaged, the position of the relative components will be maintained in the event the practitioner releases the device at some point in the process. In other words, the angled orientation of the engaged teeth will prevent premature or unwanted movement of the tamper and support tubes 442, 454 in a proximal direction until desired.

After the cap 102 has been properly positioned and tightened by the practitioner, the stopper 444 is retracted in a proximal direction. As discussed below, this proximal movement of the stopper 444 will cause the teeth (not shown) of the arm 632 to disengage from the teeth 620 of the bearing surface 625, “unlocking” the ratchet 630. For example, as can be seen in FIG. 26E, the ratchet 630 engages with teeth 620. In FIG. 26F, as the stopper 444 begins moving proximally, the stopper 444 pushes laterally against the flexible arm 632 causing it to deflect away and disengage from the teeth 620 of bearing surface 625. Specifically, the stopper 444 is forced up a ramp 635 and comes into contact with a protrusion 638. As the stopper 444 moves up the ramp 635 and mechanically engages the protrusion 638, the force enacted by the stopper 444 causes the flexible arm 632 of the ratchet 630 to flex away from and disengage teeth 620. The orientation of teeth 620 is such that they prevent proximal movement of the ratchet 630. When the flexible arm 632 of the ratchet 630 disengages teeth 620, the entire ratchet 630 can be retracted in a proximal direction, without interference from the teeth 620.

During an initial proximal portion of travel of the stopper 444 in the proximal direction, the latch assembly 600 holds the support tube 454 stationary. For example, the tamper tube 442 may be held stationary for the first approximately 6 mm (e.g., 5-8 mm) of proximal travel of the stopper 444. This action “unsheathes” the sealant 104 so it is fully released within the patient tissue tract without moving or deviating from the tissue tract. As before, the sealant 104 may have a length of approximately 5 mm.

After the initial 6 mm of proximal travel is complete, the tamper and support tubes 442, 454 move again as one to the end of proximal travel for the stopper 444. The latch assembly 600, similar to the embodiment described above and illustrated in FIGS. 24A-25C, then deploys in the last part of slider's 552 travel to free the delivery device from the patient tissue tract and release the suture 106 from the internal suture pin 520.

In some embodiments, components of the latch assemblies 500, 600 (e.g., pin 520, first and second latches 502, 504) could be restrained out of the path of the stopper 444 by a release pin (not illustrated). The release pin can be displaced when the stopper 444 compresses the cap 102 (e.g., near the distal end of the stopper's 444 stroke), releasing the latch assembly 500, 600 to be present in the stopper's 444 path. Thus, on the proximal stroke of the stopper 444, the latch assembly 500, 600 is in the path of the stopper 444 and can be tripped, allowing release of the suture 106 and disengagement of the delivery device.

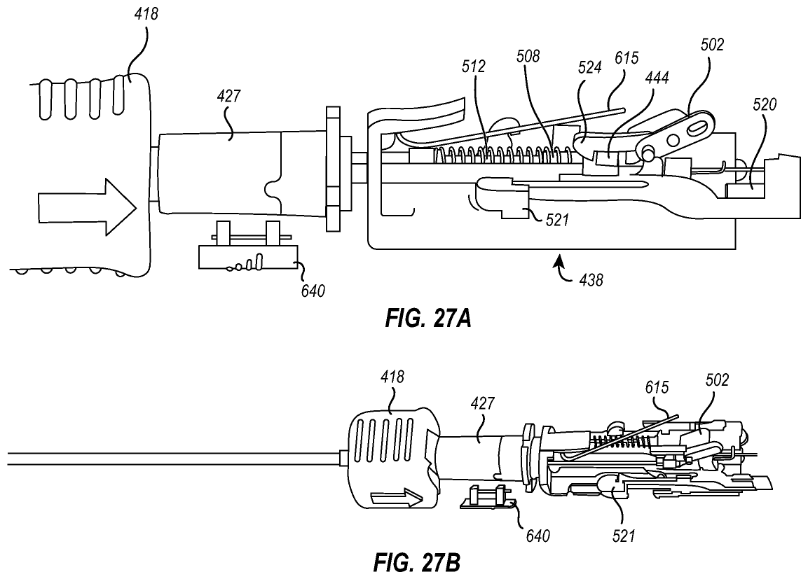

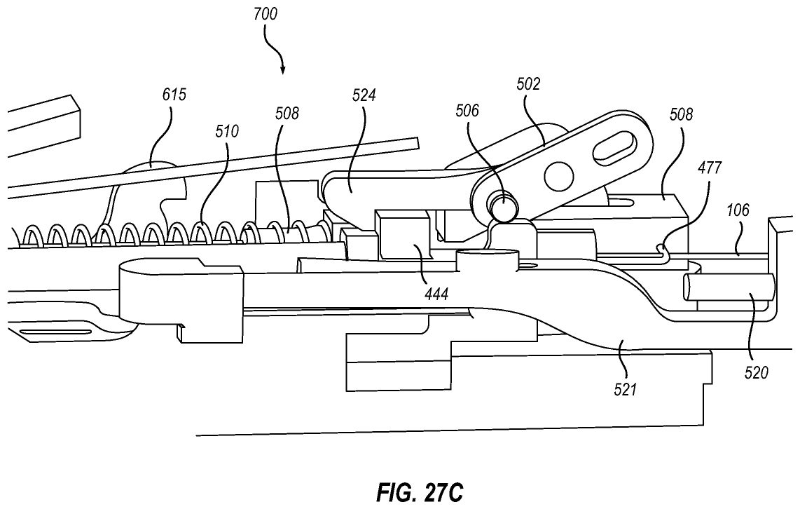

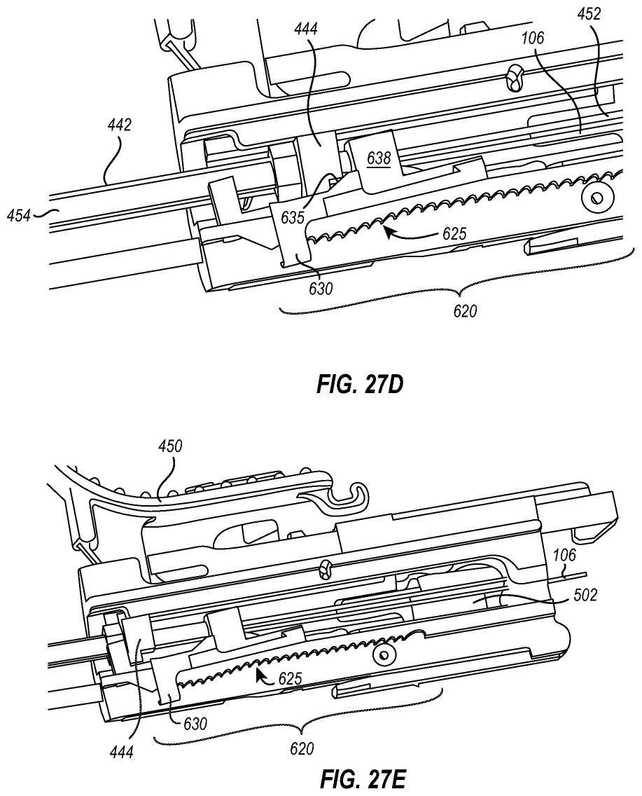

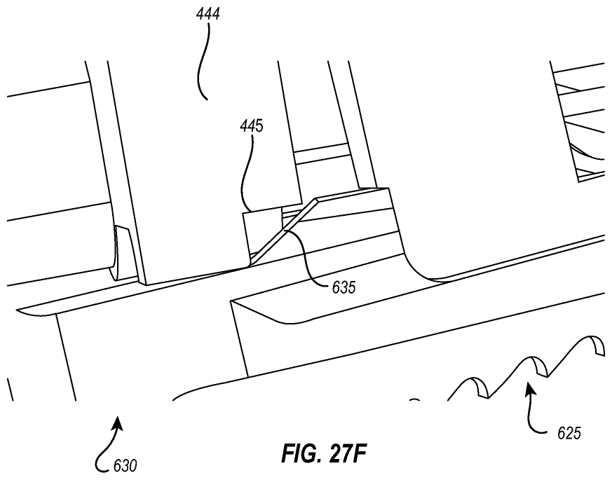

FIGS. 27A-27F illustrate another embodiment of a latch assembly 700. The disclosure and discussion related to other latch assemblies described herein are also applicable to the latch assembly 700, and so like structures are provided with like reference numerals. Similar to latch assemblies 500, 600, the components of the latch assembly 700 can be contained within carriage 438 and mechanically communicate with slider 450. As seen in FIGS. 14A, 14E-14F, and 16, the carriage 438 has a body that is grooved or otherwise shaped. In some embodiments, these grooves and/or shapes are designed and/or configured to receive and interact with various components of the latch assemblies described herein (e.g., 500-700).

As shown in FIGS. 27A-27B, another embodiment of a latch assembly 700 can include a latch 502 having a hook 524. The hook 524 is configured to receive the stopper 444 and lock the stopper 444 in place. While the latch assemblies 500, 600 described and illustrated in FIGS. 24A-26F are described as including first and second latches 502, 504, the latch assembly 700 can include a single latch 502 that incorporates (in a single piece) the functions of both the first and second latches as described in connection with FIGS. 24A-26F. The latch assembly 700 also includes a bolt body 521 that is connected to the pin 520. In some embodiments, the bolt body 521 is similar to both the first slider block 521 and the pin body 528 of latch assembly 500. The bolt body 521 of latch assembly 700 can provide similar functions of both the first slider block 521 and the pin body 528 described herein. Similar to the latch assemblies 500, 600 previously described, the suture 106 is looped around the pin 520 and is only released from the pin 520 upon proper placement of the cap 102, anchor 108, and sealant 104.

FIG. 27C illustrates a close-up view of the latch assembly 700. The hook 524 of the latch 502 can clearly be seen receiving/engaging the stopper 444, preventing the stopper 444 from being moved distally until the hook 524 has been rotated away and disengaged from the stopper 444. Also clearly illustrated is the bolt body 521 and the pin 520. Though the suture 106 is illustrated as passing past the bolt body 521, the suture 106 will loop around the pin 520. The suture 106 may be one big loop, where a middle portion of the suture 106 is looped around the pin 520, and both ends of the suture 106 are threaded through the cap 102 and secured to the anchor 108. The bolt body 521 and the pin 520 will be discussed more fully below.

The latch assembly 700 also includes a spring 510 wrapped around a spring guide 508. The spring guide 508, similar to rod 508 described with respect to latch assembly 500, is configured to receive the push wire with bend 477. As better seen in FIG. 27A, the spring 510 is also wrapped around a rod 512 that helps maintain a spring force of the spring 510. The spring guide 508, together with the spring 510 and the rod 512, set a certain amount of spring load into the suture 106. When a practitioner pulls back on the device after delivering the anchor 108, cap 102, and sealant 104, the spring force will cause the spring guide 508 to retract, pulling the push wire 452 with it (via movement of the push wire bend 477). When the spring guide 508 has retracted a set amount (and, thus, pulled the fork end 457 of the push wire 452 off of the anchor 108), the spring guide 508 will run into cars within the carriage 438 resulting in an audible “click.”

This audible feedback indicates to the practitioner that the push wire 452 has been removed from the anchor 108 and the practitioner can move on to the next step of the procedure. The spring guide 508 also interacts with the latch 502 and the hook 524. Specifically, a notch on the spring guide 508 interfaces with the latch 502. When the practitioner pulls back on the device and exerts a force on the suture 106, the latch 502 is unlocked and releases the spring guide 508. As already described, pulling back on the device and exerting the force on the suture 106 also causes the spring guide 508 to retract and bring the push wire 452 with it. The practitioner will hear audible feedback (e.g., a “click”) when the spring guide 508 has moved sufficiently proximally to remove the forked end 457 of the push wire 452 from the anchor 108. Unlocking the latch 502 also releases the stopper 444, allowing the stopper 444 to move distally to tighten the cap 102 around the blood vessel.

As seen in FIGS. 27D-27F, the stopper 444 also interacts with a ratchet 630. As seen most clearly in FIG. 27F, the ratchet 630 includes a ramp 635 that interacts with a notch 445 of the stopper 444. This ramp 635 prevents the stopper 444 from being moved proximally. As the carriage 438 is moved distally, the ratchet 630 is moved along an array of teeth 620 (see FIG. 27D-27E). The ratchet 630 and the stopper 444 move together as one during distal movement of the carriage 438. When the carriage 438 has finished its distal stroke, the stopper 444 deploys the cap 102 and the sealant 104.

The array of teeth 620 includes a ramp portion 625. When the first slider block 630 is moved distally along the array of teeth 620 and past the ramp portion 625, the ramp 635 is cleared out of a proximal path of the stopper 444. The stopper 444 can then be moved proximally and “up” the ramp 635. The stopper 444 will hit a protrusion 638 of the ratchet 630; the stopper 444 and the ratchet 630 will move proximally as one. Moving the stopper 444 (and the ratchet 630) proximally unsheathes the sealant 104 by moving the tamper tube 442 proximally.

The sealant 104 stays in place within the tissue tract (and positioned against the cap 102) while the tamper tube 442 is moved proximally. Unsheathing the sealant 104 in this way, while keeping the sealant 104 in place within the tissue tract, exposes the sealant 104 to blood and other bodily fluids within the tissue tract. The sealant 104 can then react with and absorb these fluids, thereby expanding within and coagulating the tissue tract.

As briefly mentioned, the suture 106 is looped around the pin 520 to prevent premature release of the suture 106 from the device, such as before the anchor 108, cap 102, and/or sealant 104 have been properly positioned and sandwiched around the vessel wall. As clearly seen in FIG. 27C, the pin 520 is attached to and an integrated part of the bolt body 521. After the stopper 444 has disengaged the ratchet 630 from the array of teeth 620, the stopper 444 and ratchet 630 can move proximally, as one. As mentioned briefly, the proximal movement of the stopper 444 and the ratchet 630 unsheathes the sealant 104 from the tamper tube 442. The proximal movement of the stopper 444 and the ratchet 630 also releases the suture 106 from the pin 520.

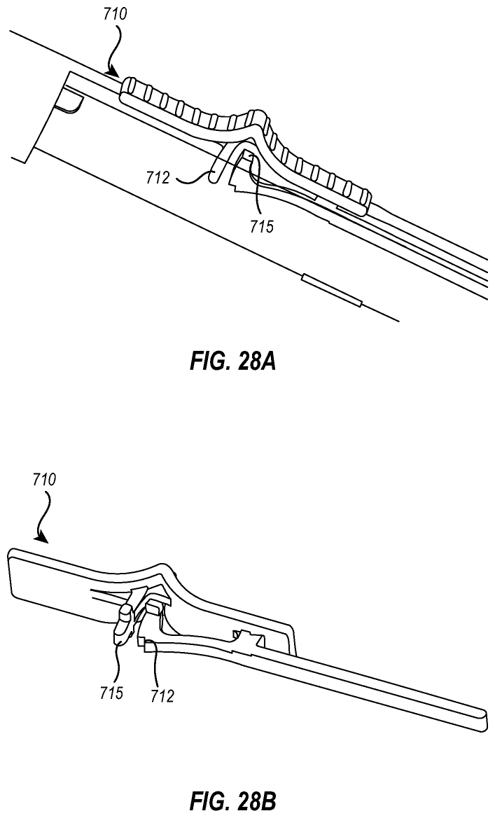

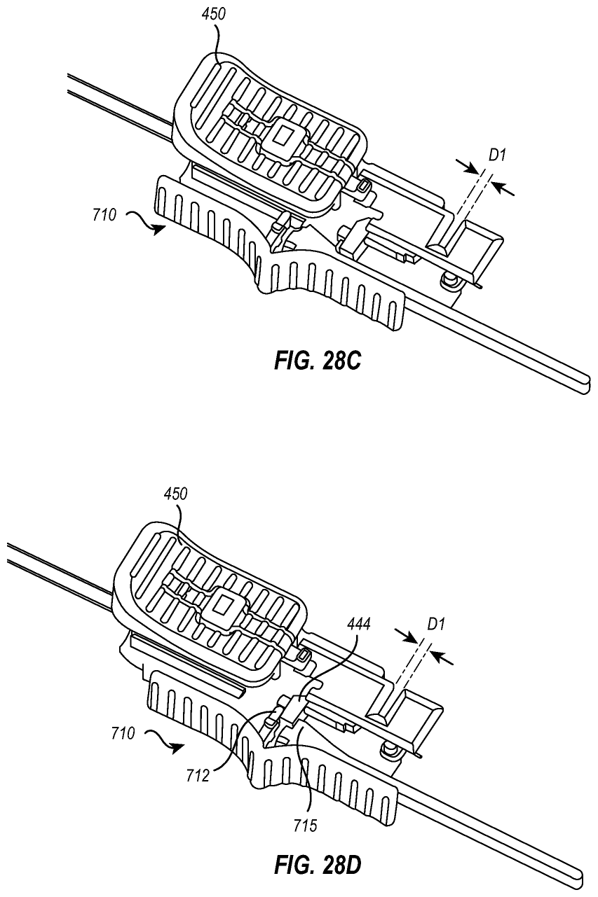

Specifically, the stopper 444 will engage a wing of the bolt body 521 such that further proximal movement of the stopper 444 also moves the bolt body 521 proximally. Referring briefly to FIGS. 28C-28D, proximal movement of the bolt body 521 clears the pin 520 from a channel having a distance D1. When the pin 520 has cleared the channel, the suture 106 can be slid off the pin 520, thereby releasing the suture 106 from the device. The practitioner may then remove the device from the patient and trim the suture 106 to an appropriate length. For example, as already described, the practitioner can depress the patient's skin/tissue and trim the suture 106 so that it rests just below the skin/tissue.

In some embodiments, the distance D1 of the channel is approximately 0.050 inch. The distance D1 can range from approximately 0.040 to 0.060 inch, such as 0.045, 0.048, 0.055, 0.058 inch, or a distance falling within a range defined by any two of the foregoing values. In some embodiments, the channel having the distance D1 is formed as part of the carriage 438. As mentioned elsewhere, the carriage 438 can have grooves or other shapes to accommodate, interface, and/or interact with various components of the disclosed latch assemblies.

FIGS. 28A-28D illustrate an alternative embodiment of a slider, which may be similar to slider 522 (FIGS. 24A-25C) or cap slide 460 (FIGS. 13A and 14A). FIG. 28A illustrates the slider 710 integrated into a distal end of a handle assembly, such as handle assembly 400 described elsewhere. The slider 710 is interlocked with the handle. As seen in FIG. 28B, the slider 710 has two latches or fingers 712 and 715. The fingers 712, 715 are configured to engage with internal elements of the latch assemblies 500, 600 and/or 700.

Once the stopper 444 has been moved distally, it flexes finger 712 and is sandwiched between fingers 712 and 715. Not only does finger 712 allow the slider 710 to apply a distal force to the stopper 444, it also has a geometry which (in its initial state) locks the slider 710 to the handle body 402. When flexed, the interlocking geometry of the slider 710 flexes out of the handle interlocking geometry. The flexed element (e.g., finger 712) is not allowed to return to its interlocked geometry/position, thereby keeping the slider 710 (and the handle assembly 400) unlocked. This prevents the practitioner from moving the slider 710 until the slider 450 has been moved/advanced distally a predetermined distance. It is noted that while reference is made to separate sliders 450 and 710 (and other sliders, slides, buttons, etc. having similar or related movements and functions), it will be understood that the functions and operations associated with the sliders 450 and 710 (and other sliders, slides, buttons, etc. having similar or related movements and functions) can be combined into any number of sliders, slides, buttons, etc. (e.g., a single slide, dual slides, more than 2 slides, etc). Therefore, it will be understood that one or more sliders, slides, buttons, etc. can be used to perform the various functions and movements described herein. For instance, and not by way of limitation, one slider can perform all the functions and operations of the sliders 450 and 710 (or other sliders, slides, buttons, etc. having similar or related movements and functions.)

For example, as seen in FIG. 28C, the carriage 438 (which moves the stopper 444) is in its final distal position, where the stopper 444 is disposed distal of either finger 712, 715. There is an interference between the finger 712 and the stopper 444. When the stopper 444 is moved proximally, the stopper passes finger 712 to be disposed between the fingers 712, 715.

### Alternative Delivery Systems

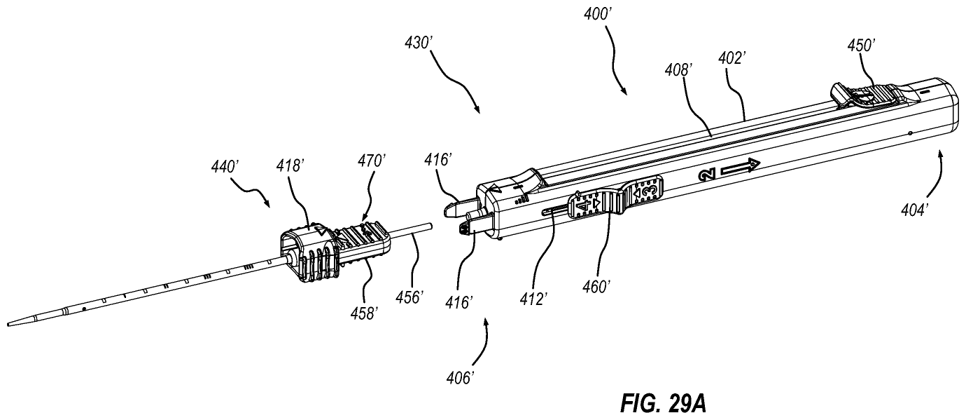

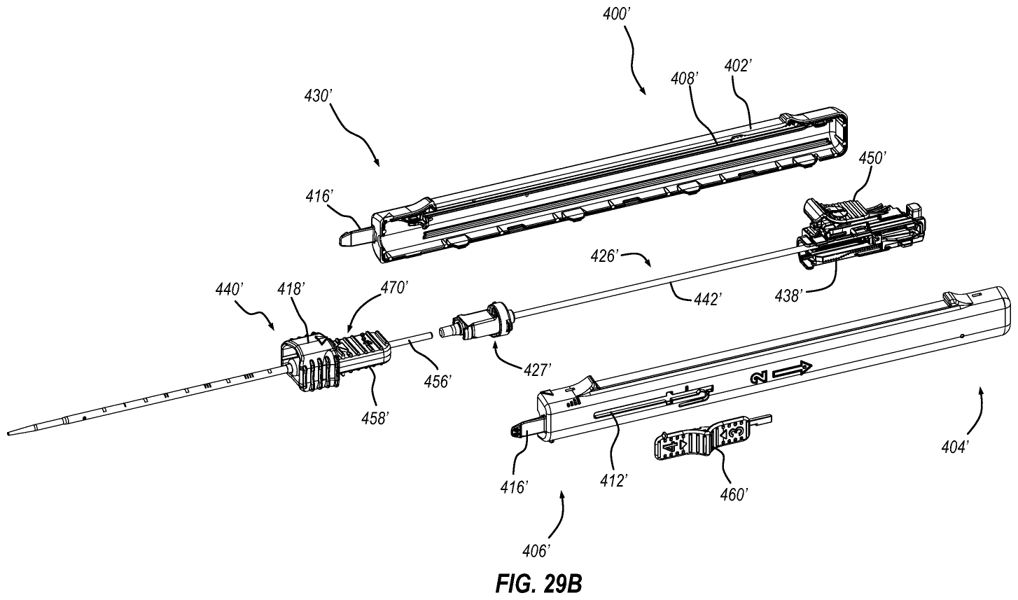

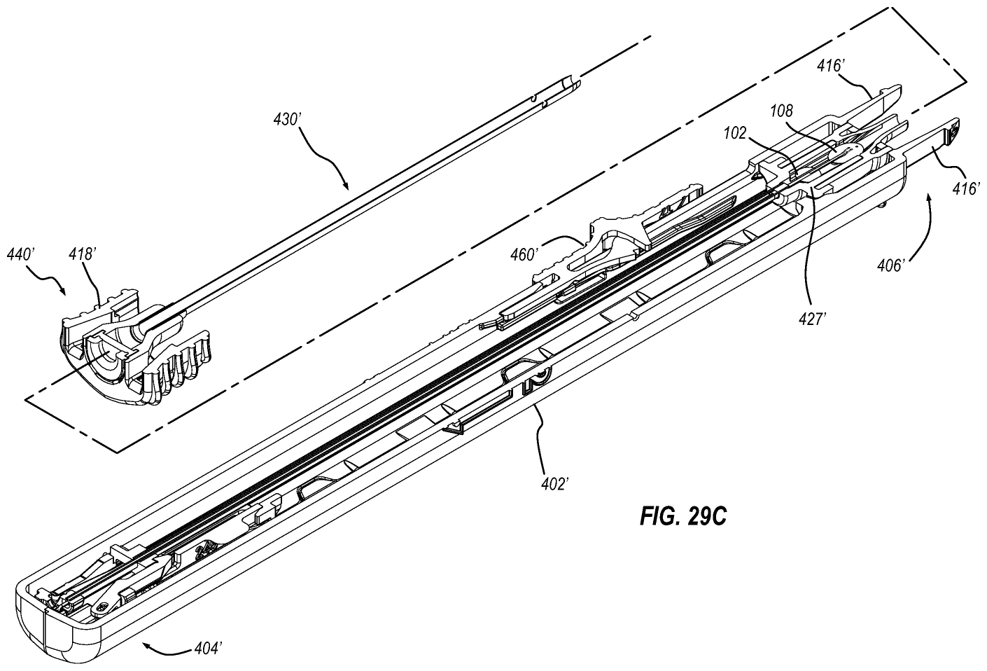

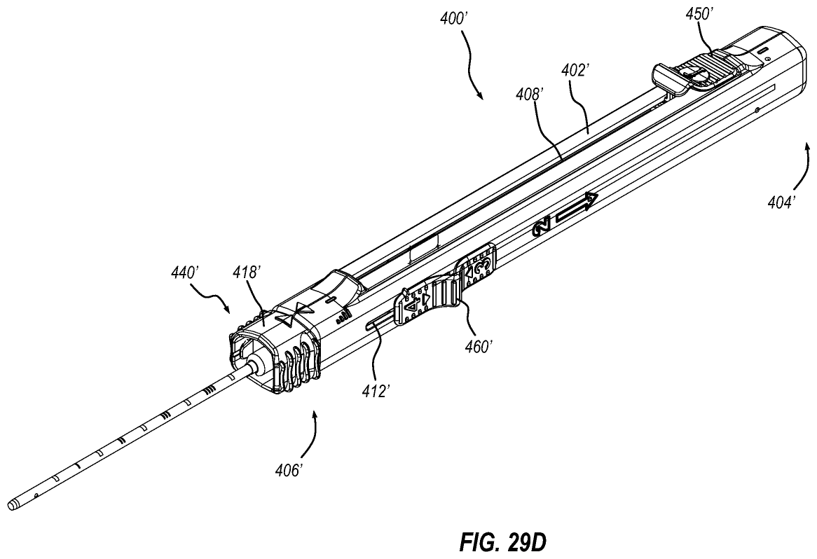

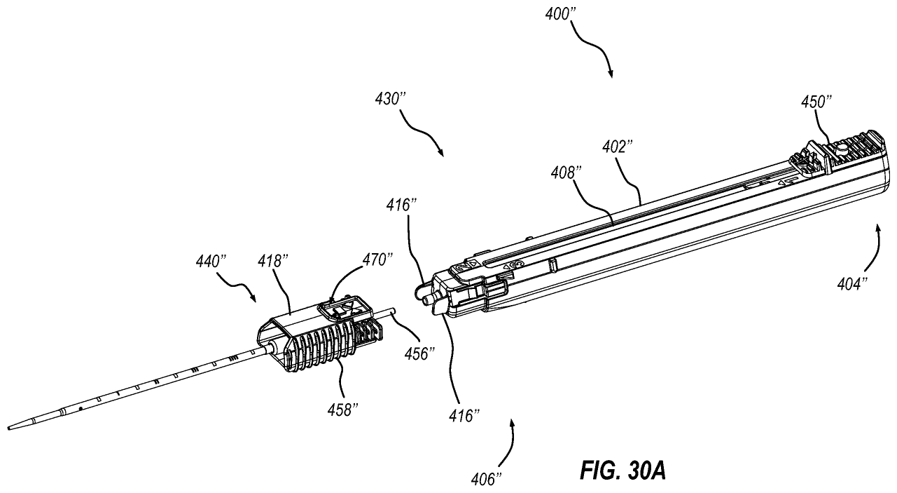

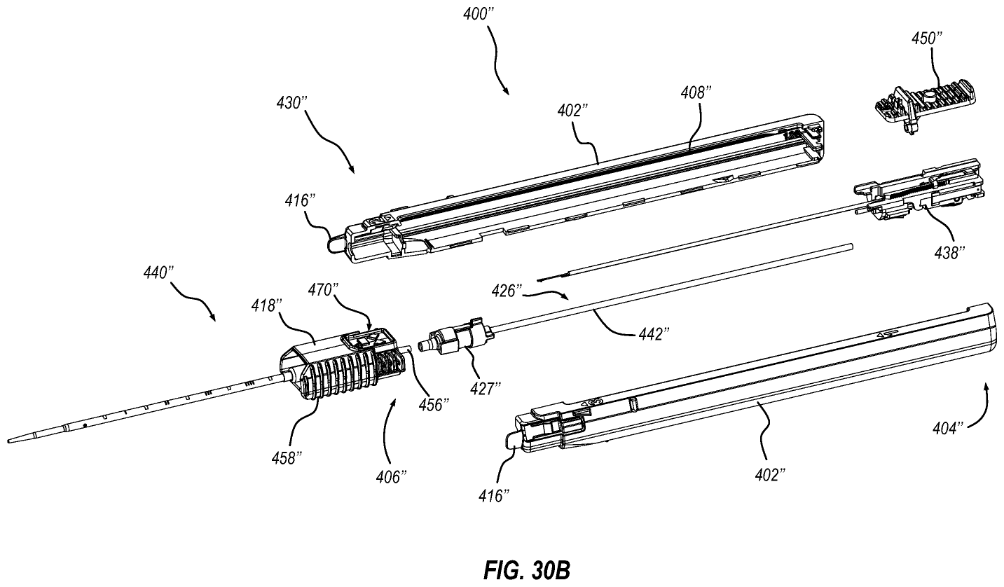

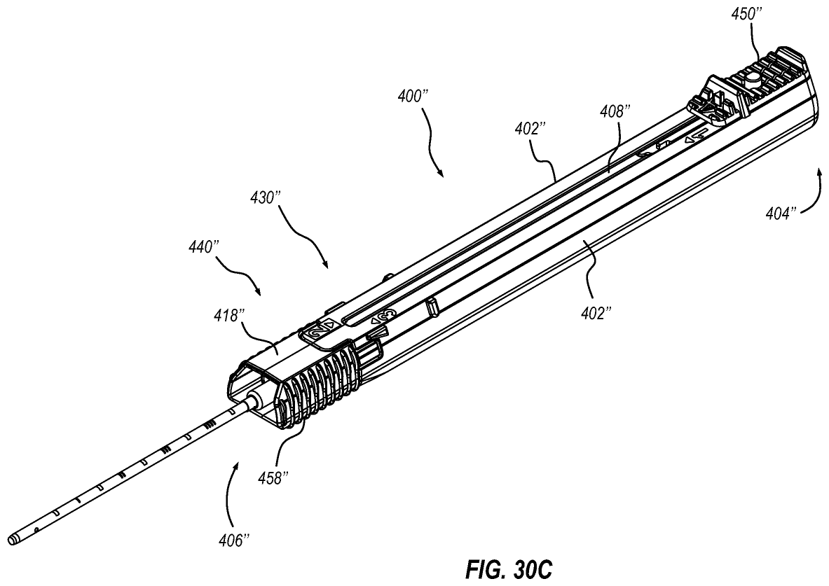

FIGS. 29A-29D and 30A-30C illustrate additional alternative delivery systems. FIGS. 29A-29D illustrate another dual slide delivery system similar to those shown and described in conjunction with FIGS. 12-28D. FIGS. 30A-30C illustrate a similar delivery system, but which is configured with only a single slide.

For example, delivery system 430′ may otherwise be similar to delivery system 430, and similar reference numerals are therefore used. The delivery system 430′ can include a handle assembly 400′ and a delivery sheath 440′. The handle assembly 400′ can be configured to be selectively attached to delivery sheath 440′. Once attached to the delivery sheath 440′, the handle assembly 400′ can be used to insert a closure device, such as, for example, closure device 100.

The handle assembly 400′ can include a handle body 402′ having a proximal end 404′ and a distal end 406′, one or more actuators (such as slider 450′ and slider 460′), and an elongate opening (e.g., openings 408′ and 412′) configured to provide a track for such sliders. The slider 450′ can slide along the elongate opening 408′ when engaged by a practitioner and be selectively locked in place by a locking assembly in a similar manner as described relative to system 430. Sliding or moving the slider 450′ along the elongate opening 408′ can deploy the closure device 100. As described relative to that previous embodiment, engagement of the slider 450′ can deploy the anchor 108, and then engagement of the cap slide 460′ can deploy the cap 102.

The handle assembly 400′ is shown as similarly including a connecting member 416′ at the distal end 406′ of the handle body 402′. The connecting member 416′ allows selective attachment of the handle body to sheath hub 418′ of delivery sheath 440′. Similar to delivery system 430, delivery system 430′ is shown as provided with a dilator assembly 470′ having a dilator tube 456′ and a dilator hub 458′.

Similar to delivery system 430′, an implant assembly 426′ is provided, which includes a closure device such as closure device 100, a tamper tube 442′, and carriage 438′. Chamber assembly 427′ analogous to chamber assembly 427 is also provided. Chamber assembly 427′ is shown somewhat differently configured, as shown. As shown in FIG. 29C, an internal structure of chamber assembly 427′ may be configured with a funnel type structure, so as to cause the anchor 108 and/or cap 102 to fold inward, over themselves, as they are advanced from the wider width end of the funnel chamber, towards the narrower width (e.g., diameter) associated with the distal end of the chamber assembly 427′, as will be apparent. This allows the anchor 108 and cap 102 to be advanced through the associated tubing in a folded configuration, where the anchor and cap can unfold, once exiting such tube. When using delivery system 430′, the procedural steps may be very similar to as described relative to system 430. Referring to the numerals 1-4 and associated directional arrows provided on handle 400′, the practitioner may first advance slider 450′ forward (numeral 1) to advance closure device 100 relative to housing body 402′ For example, advancing slider 450′ forward advances the implant components forward to a stop position where an audible and tactile click occurs and the anchor is fully ejected from the sheath. Then, the practitioner may move the entire handle body 402′ slightly rearward, as indicated by numeral 2 and the associated directional arrow to pull anchor 108 against the vessel wall For example, moving the entire handle body 402′ slightly rearward pulls the anchor 108 up against the inner vessel wall until the force of the apposition against the vessel wall and along the suture is enough to trip the interlock latch. Tripping the interlock latch unlocks the side slide 460′ and also releases a spring guide that causes the push wire fork to retract until the fork is proximal to the sealant material located in the tamper tube 442′. Then the practitioner may push slider 460′ forward as indicated by numeral 3 and the associated directional arrow to deploy cap 102. For example, pushing slide 460′ forward advances the tamper tube 442′ and support tube 454 positioned therein, which in turn advances the cap 102 until it stops against the outer vessel wall, while the device handle is holding the anchor seal against the inner wall of the vessel by maintaining tension on the suture. This may be followed by pulling slider 460′ rearward as indicated by numeral 4 and the associated directional arrow to unsheathe the sealant, and then release the suture 106. In particular, pulling slider 460′ rearward will first unsheathe the sealant material located inside the tamper tube 442′ while the sealant component is held in place on top of cap seal 102 by support tube 454 (e.g., see FIG. 24N). Continued pulling rearward of slider 460′ will secondly cause rearward movement of the suture latch so that the pin located in the suture latch will disengage from the suture loop. In some instances, it may or may not move the support tube rearward.

Delivery system 430″ of FIGS. 30A-30B may otherwise be similar to delivery system 430 and others described herein, and similar reference numerals are therefore used. The delivery system 430″ can include a handle assembly 400″ and a delivery sheath 440″. The handle assembly 400″ can be configured to be selectively attached to delivery sheath 440″. Once attached to the delivery sheath 440″, the handle assembly 400″ can be used to insert a closure device, such as, for example, closure device 100.

The handle assembly 400″ can include a handle body 402″ having a proximal end 404″ and a distal end 406″, an actuator (such as single slider 450″), and an elongate opening 408″ configured to provide a track for such sliders. The slider 450″ can slide along the elongate opening 408″ when engaged by a practitioner. Slide 450″ may serve multiple functions, e.g., to deploy the closure device 100, as well as to pull anchor 108 against the vessel wall, and deploy cap 102.

The handle assembly 400″ is shown as similarly including a connecting member 416″ at the distal end 406″ of the handle body 402″. The connecting member 416″ allows selective attachment of the handle body to sheath hub 418″ of delivery sheath 440″. Similar to delivery system 430, delivery system 430″ is shown as provided with a dilator assembly 470″ having a dilator tube 456″ and a dilator hub 458″.

Similar to delivery system 430″, an implant assembly 426″ is provided, which includes a closure device such as closure device 100, a tamper tube 442″, and carriage 438″. Chamber assembly 427″ analogous to chamber assembly 427 is also provided. When using delivery system 430″, the procedural steps may include first advancing slider 450″ forward (numeral 1) to a first stop position, to advance closure device 100 relative to housing body 402″. When first advancing slider 450″ the forward and downward pressure on the slider will unlock the slider from attachment to the handle body, allowing the slider to move forward freely. Once advanced, the practitioner may pull the entire handle body 402″ slightly rearward, as indicated by numeral 2 and the associated directional arrow to pull anchor 108 against the vessel wall. Then the practitioner may push slider 450″ further forward to a second stop position as indicated by numeral 3 and the associated directional arrow to deploy cap 102. Pushing slider 450″ forward to such a stop position will deploy the cap 102 to tightly sandwich the vessel between the anchor and cap. This may be followed by pulling slider 450″ rearward as indicated by numeral 4 and the associated directional arrow to unsheathe the sealant, and release the suture 106. In particular, pulling slider 450″ rearward will unsheathe the sealant material on top of the cap and further rearward movement to a stop position will release the suture.

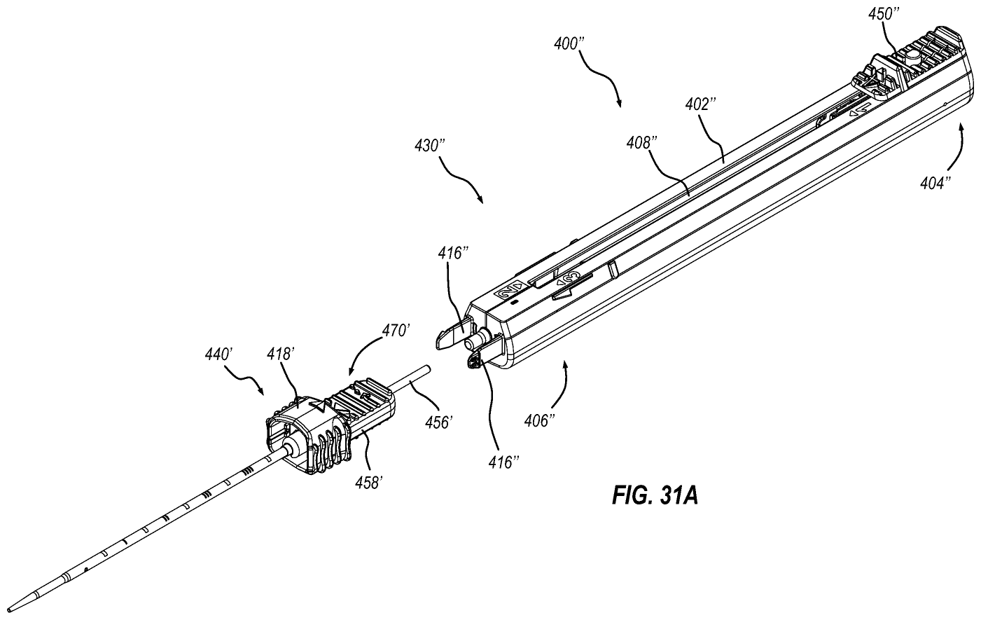

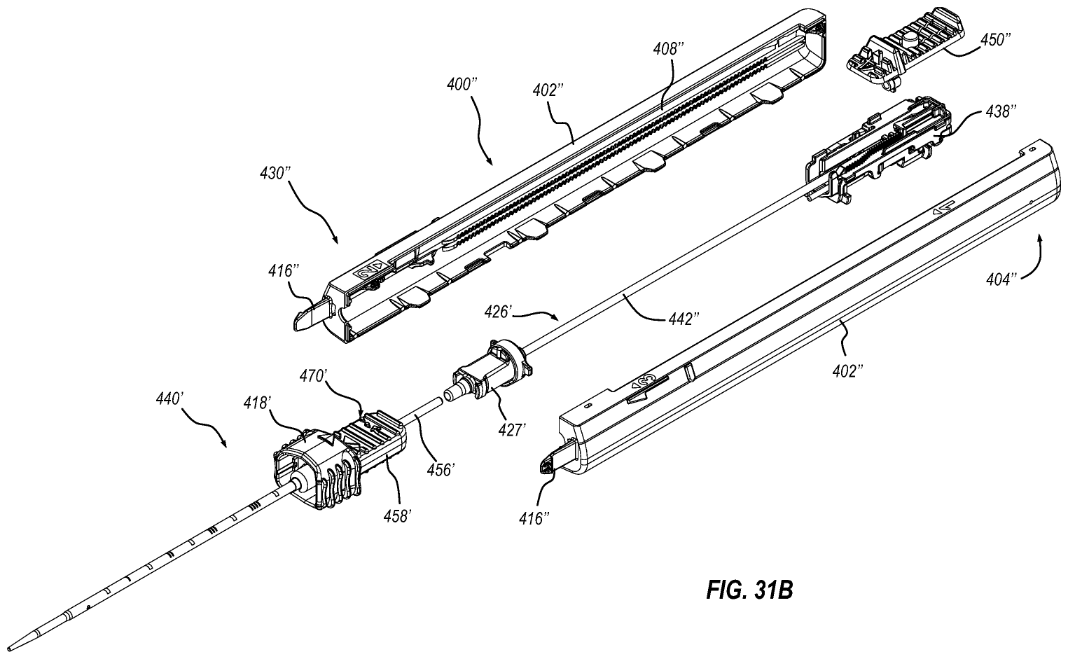

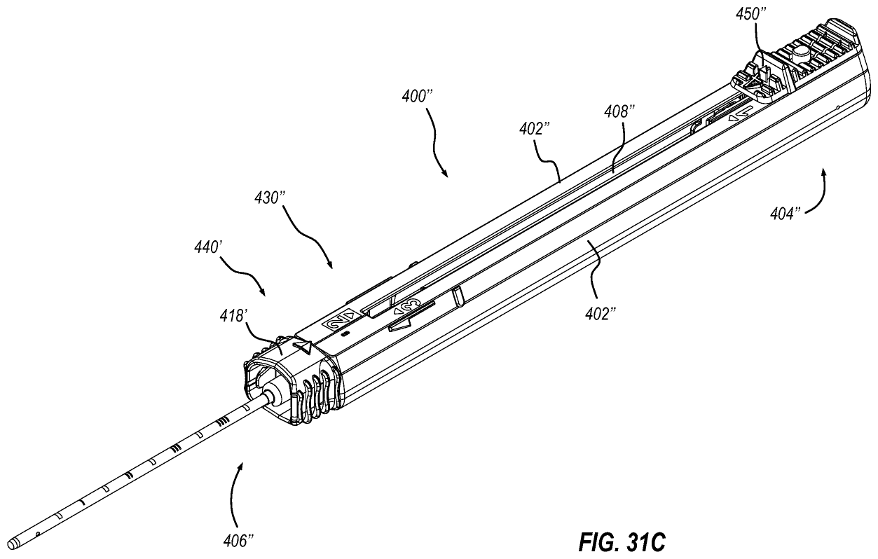

FIGS. 31A-31C show a single slider configuration similar to that shown in FIGS. 30A-30C, but where the sheath hub, delivery sheath, dilator hub, dilator assembly, and dilator tube are similar to the delivery system shown in FIGS. 29A-29D, which includes dual slides.

It will be appreciated that a variety of configurations and associated procedural steps are possible, to advance the closure device 100 into the body lumen, to pull anchor the 108 against the vessel wall, to deploy the cap 102, and finally to release the suture 106 after the closure device 100 has closed the puncture access site 18.

Method of Closure Device Insertion with Handle Assembly Having Latch Assembly

In order to deliver a closure device 100 using the handle assembly 400 having a latch assembly 500, 600, or 700, first the dilator tube 456 can be inserted into the delivery sheath 440. The dilator tube 456 can be selectively attached to the sheath 441 by connecting the dilator hub 458 to the sheath hub 418 in order to maintain the position of the dilator tube 456 in the delivery sheath 440 (FIGS. 20A-20B). The dilator tube 456 can be used to stretch the opening of the skin 16 and access tract 22 to allow for placement of a closure device 100.

Next, the dilator hub 458 can be disengaged from the sheath hub 418 and the dilator tube 456 can be removed, as shown in FIG. 21B. The sheath 441 can remain in the access tract 22. FIGS. 21C and 21D illustrate a method of connecting the handle assembly 400 to the delivery sheath 440. The handle assembly 400 can be selectively connected to the delivery sheath 440 by engaging the connecting members 420 of the handle assembly 400 with the receiving member 468 of the sheath hub 418 of the delivery sheath 440.

Once the handle assembly 400 is connected to the delivery sheath 440, the user can depress the proximal locking assembly 421 to unlock the slider 450 and push the slider 450 in a distal direction towards the distal end 406 of the handle body 400, as illustrated in FIG. 22. This causes the delivery system 430 to eject the anchor 108 into the blood vessel lumen 12 so that the anchor 108 can contact the inner vessel wall 14A and be positioned on the puncture site 18. Once the slider 450 reaches the distal end 406, the anchor 108 should be ejected from the delivery system 430, with the cap 102 and sealant 104 remaining in the tamper tube 442 of the implant assembly 426 within the delivery sheath 440.

Turning to FIG. 23A, the slider 450 can be configured to slide along the elongate opening 408 until slider 450 slides distally past locking assembly 425, at which point locking assembly 425 can lock slider 450 at the distal end of elongate opening 408. The locking assembly 425 can be formed with the handle body 402, such as having a living hinge connection with the handle body 402 or can be a separated.

By moving the slider 450 past the locking assembly 425, the engagement of the locking assembly 425 can actuate/trip the latch assembly 500, causing the first latch 502 to engage the stopper 444 and lock the stopper 444 (and in effect the implant assembly 426) in place within the delivery sheath 440. The engagement of the first latch 502 can actuate the second latch 504, causing the second latch 502 to be moved into a locked position to selectively retain the push wire bend 477 of the push wire 452 of the implant assembly 426.

Next, the practitioner can pull back on the handle assembly 400 along the axis of the device (e.g., at a 45-degree angle relative to the tissue tract 22) to tension the suture 106 and ensure proper placement of the anchor 108. When tension is applied to the suture 106 by the practitioner, the suture loop 526 looped around the pin 520 causes the suture 106 to apply downward pressure on the first latch 502, releasing the stopper 444 from the first latch 502. The release of the first latch 502 can cause the second latch 504 to release the push wire bend 477, allowing the spring 510 to decompress and push the rod 508 in a proximal direction. The proximal movement of the rod 508 causes the push wire 452 to retract from the closure device 100, so that the forked end 457 of the push wire 452 is at least proximal of the proximal end of the sealant 104, so as not to interfere with the placement of the sealant 104.

When the spring 510 is released, it can pull the push wire 452 back into tamper tube 442. The user can feel the spring 510 actuate. The slider 522 and tamper slider 528 may not move to tamp the cap 102 until the spring 510 is actuated. This mechanism can ensure that the anchor 108 is optimally sealed against the inner wall of the artery.

The slider 522 can then engage the tamper slider 528 to tamp the cap 102 to ensure a secure fit. Next, the slider 522 can be drawn in proximal direction to draw the support tube 454 in a proximal direction to passively unsheathe the fluid blocking component 104. A tamper tube 442 can be locked in position proximal to the fluid blocking component 104 to maintain the position of the fluid blocking component 104 while it is being placed. The tamper tube can be retracted in a proximal direction by activation of the slider from about 4 mm to about 8 mm, from about 5 mm to about 7 mm, or from about 5.5 mm to about 6.5 mm. The support tube 454 can be retracted so that it does not interfere with placement of the fluid blocking component 104.

When the support tube 454 is retracted by engaging slider 522, the slider 522 can cause the pin 520 to release the suture loop 526, thereby releasing the suture 106 and closure device 100 from the delivery system 430.

In some embodiments, a method of deploying or inserting a closure device using a disclosed handle assembly includes advancing a delivery sheath and/or a dilator over a wire. The method also includes checking a blood mark, and removing the dilator and wire. The method further includes connecting a closure device to the delivery sheath and engaging a slider to deliver an anchor inside a vessel. Additionally, the method may include gently pulling the handle assembly back (approximately 2 cm) until a second slider (e.g., a cap slide) is unlocked. A practitioner will hear and feel a click from a locking mechanism, indicating the second slider is unlocked.

The method includes advancing the second slider to push a cap toward the vessel, and tightening the closure device around the vessel wall. After tightening the closure device, a state of hemostasis should be evaluated and, if necessary, additionally tightening of the closure device should occur. Such tightening can occur through proximally tugging or pulling of the closure device and/or a handle attached to the delivery sheath. The method also includes retracting the second slider until a detent click is felt and the device is removed.

### Additional Terms/Definitions

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Various values and ranges are disclosed herein. Additional ranges may be defined between any values disclosed herein as being exemplary of a particular parameter. All such ranges are contemplated and within the scope of the present disclosure.

Any directions or reference frames described herein are merely relative directions (or movements). For example, any references to “top”, “bottom”, “up” “down”, “above”, “below” or the like are merely descriptive of the relative position or movement of the related elements as shown, and it will be understood that these may change as the structure is rotated, moved, the perspective changes, etc.

Following are some further example embodiments of the invention. These are presented only by way of example and are not intended to limit the scope of the invention in any way. Further, any example embodiment can be combined with one or more of the example embodiments.

Embodiment 1. A vessel closure device including a bioabsorbable vessel closure device for delivering substantially immediate hemostasis at a puncture site in a wall of a blood vessel, the closure device including an intravascular anchor comprising one or more suture attachment points, an extravascular cap comprising a lumen, a sealant, and a suture connected to at least one of the one or more suture attachment points of the intravascular anchor and threaded through the lumen of the extravascular cap and through the sealant to connect the intravascular anchor to the extravascular cap and to the sealant, wherein each of the intravascular anchor, extravascular cap, sealant, and suture are formed of bioabsorbable materials.

Embodiment 2. The vessel closure device of embodiment 1, wherein the intravascular anchor includes an elongate body comprising a flexible member and a keel.

Embodiment 3. The vessel closure device of any of embodiment 1-2, wherein the extravascular cap is formed of a flexible material.

Embodiment 4. The vessel closure device of any of embodiment 1-3, wherein the sealant comprises polyethylene glycol (PEG).

Embodiment 5. The vessel closure device of any of embodiment 1-4, wherein the suture comprises a distal suture portion and a proximal suture portion.

Embodiment 6. The vessel closure device of any of embodiment 1-5, wherein the diameter of the lumen of the extravascular cap is smaller than the diameter of the distal suture portion.

Embodiment 7. The vessel closure device of any of embodiment 1-6, wherein the intravascular anchor comprises a bioabsorbable material.

Embodiment 8. The vessel closure device of any of embodiment 1-7, wherein the intravascular anchor comprises a plurality of ribs radiating from the keel to a raised edge of the elongate body.

Embodiment 9. The vessel closure device of any of embodiment 1-8, wherein the sealant can expand up to 4 times its original size when introduced to fluids.

Embodiment 10. A vessel closure device for delivering substantially immediate hemostasis at a puncture site in a wall of a blood vessel, the closure device including an intravascular anchor comprising one or more suture attachment points, an extravascular cap comprising a lumen, a sealant comprising a lumen, and a suture connected to at least one of the one or more suture attachment points of the intravascular anchor and threaded through the lumen of the extravascular cap and through the lumen of the sealant to connect the intravascular anchor to the extravascular cap and to the sealant, wherein the suture comprises a proximal suture portion and a distal suture portion, wherein the distal suture portion has a diameter greater than a diameter of the lumen of the extravascular cap, wherein the distal suture portion creates an interference fit to lock the extravascular cap over the puncture site, wherein each of the intravascular anchor, extravascular cap, sealant, and suture are formed of bioabsorbable materials.

Embodiment 11. The vessel closure device of any of embodiment 10, wherein the extravascular cap is formed of a flexible material.

Embodiment 12. The vessel closure device of any of embodiment 10-11, wherein the suture is a braided suture.

Embodiment 13. The vessel closure device of any of embodiment 10-12, wherein the sealant is threaded onto the suture at a location proximal to the extravascular cap.

Embodiment 14. The vessel closure device of any of embodiment 10-13, wherein the sealant when activated locks the extravascular cap in place and coagulates an access tract of the puncture site providing substantially immediate hemostasis.

Embodiment 15. The vessel closure device of any of embodiment 10-14, wherein the intravascular anchor comprises an elongate body comprising a flexible member.

Embodiment 16. The vessel closure device of any of embodiment 10-15, wherein the intravascular anchor comprises a raised keel located on a central axis of the elongate body and spanning the length of the elongate body.

Embodiment 17. The vessel closure device of any of embodiment 10-16, wherein the raised keel comprises one or more suture attachment points.

Embodiment 18. The vessel closure device of any of embodiment 10-17, wherein the sealant comprises polyethylene glycol (PEG).

Embodiment 19. An intravascular anchor for a vessel closure device for delivering substantially immediate hemostasis at a puncture site in a wall of a blood vessel, the intravascular anchor includes an elongate body including a flexible member for conforming to the wall of the blood vessel, a keel having one or more suture attachment points, wherein the keel is an elongate member centrally located along a central axis of the elongate body.

Embodiment 20. The intravascular anchor of claim 19, wherein the elongate body includes a plurality of ribs radiating from the keel to a raised edge forming the perimeter of the elongate body.

Embodiment 21. A delivery system for delivering a vessel closure device to provide substantially immediate hemostasis at a puncture site in a wall of a blood vessel, the delivery system comprising a handle assembly, the handle assembly comprising a body having a proximal end, a distal end, a lumen extending from the proximal end to the distal end, a first actuator and an elongate opening configured to provide a track for the first actuator, an implant assembly situated in the lumen of the body, the implant assembly comprising a vessel closure device, a slider and a chamber, wherein the slider of the implant assembly is configured to be engageable with the first actuator, and an interlock mechanism configured to selectively disengage the delivery system from the vessel closure device upon delivery of the vessel closure device to the puncture site in the wall of the blood vessel; a delivery sheath configured to be selectively coupled to the distal end of the handle assembly, wherein the vessel closure device is configured to be delivered through the delivery sheath to the puncture site in the wall of the blood vessel and secured around the puncture site, thereby providing substantially immediate hemostasis.

Embodiment 22. The delivery system of embodiment 21, wherein the interlock mechanism comprises a first latch, a second latch connected to the first latch via a latch pin, a bolt for securing at least a portion of the proximal suture portion of the suture of the vessel closure device; and a slider in mechanical communication with the bolt, wherein the slider is configured to release the at least a portion of the proximal suture portion of the suture, and wherein release of the at least a portion of the proximal suture portion of the suture disengages the delivery system from the vessel closure device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. It shall be further understood that although the present invention has been described in relation to vessel closure, it is contemplated that the closure component of the present invention may be utilized to close other openings in the body such as PFO openings, or openings formed in organs such as the stomach for certain surgical procedures.

What is claimed is:

1. A vessel closure device for delivering substantially immediate hemostasis at a puncture site in a wall of a blood vessel, the closure device comprising:
an intravascular anchor comprising one or more suture attachment points;
an extravascular cap comprising a lumen;
a sealant; and
a suture connected to at least one of the one or more suture attachment points of the intravascular anchor and threaded through the lumen of the extravascular cap and through the sealant to connect the intravascular anchor to the extravascular cap and to the sealant,
wherein each of the intravascular anchor, extravascular cap, sealant, and suture are formed of bioabsorbable materials.

2. The vessel closure device of claim 1, wherein the intravascular anchor comprises an elongate body comprising a flexible member and a keel.

3. The vessel closure device of claim 1, wherein the extravascular cap is formed of an elastomeric material.

4. The vessel closure device of claim 1, wherein the sealant comprises polyethylene glycol (PEG).

5. The vessel closure device of claim 1, wherein the suture comprises a distal suture portion and a proximal suture portion.

6. The vessel closure device of claim 5, wherein a diameter of the lumen of the extravascular cap is smaller than a diameter of the distal suture portion.

7. The vessel closure device of claim 1, wherein the intravascular anchor comprises a bioabsorbable material.

8. The vessel closure device of claim 1, wherein the intravascular anchor comprises a plurality of ribs radiating from a keel to a raised edge of the elongate body.

9. The vessel closure device of claim 1, wherein the sealant can expand up to 4 times its original size when introduced to fluids.

10. A vessel closure device for delivering substantially immediate hemostasis at a puncture site in a wall of a blood vessel, the closure device comprising:
an intravascular anchor comprising one or more suture attachment points;
an extravascular cap comprising a lumen;
a sealant comprising a lumen; and
a suture connected to at least one of the one or more suture attachment points of the intravascular anchor and threaded through the lumen of the extravascular cap and through the lumen of the sealant to connect the intravascular anchor to the extravascular cap and to the sealant,
wherein the suture comprises a proximal suture portion and a distal suture portion, wherein the distal suture portion has a diameter greater than a diameter of the lumen of the extravascular cap;
wherein the distal suture portion creates an interference fit to lock the extravascular cap over the puncture site;
wherein each of the intravascular anchor, extravascular cap, sealant, and suture are formed of bioabsorbable materials.

11. The vessel closure device of claim 10, wherein the extravascular cap is formed of flexible material.

12. The vessel closure device of claim 11, wherein the sealant when activated locks the extravascular cap in place and coagulates an access tract of the puncture site providing substantially immediate hemostasis.

13. The vessel closure device of claim 10, wherein the suture is a braided suture.

14. The vessel closure device of claim 10, wherein the sealant is threaded onto the suture at a location proximal to the extravascular cap.

15. The vessel closure device of claim 10, wherein the intravascular anchor comprises an elongate body comprising a flexible member.

16. The vessel closure device of claim 15, wherein the intravascular anchor comprises a raised keel located on a central axis of the elongate body and spanning a length of the elongate body.

17. The vessel closure device of claim 16, wherein the raised keel comprises one or more suture attachment points.

18. The closure device of claim 10, wherein the sealant comprises polyethylene glycol (PEG).

19. A intravascular anchor for a vessel closure device for delivering substantially immediate hemostasis at a puncture site in a wall of a blood vessel, the intravascular anchor comprising:
an elongate body comprising a flexible membrane for conforming to the wall of the blood vessel; and
a keel having one or more suture attachment points;
wherein the keel is an elongate member centrally located along a central axis of the elongate body;
wherein the intravascular anchor comprises a bioabsorbable material.

20. The intravascular anchor of claim 19, wherein the elongate body comprises a plurality of ribs radiating from the keel to a raised edge forming a perimeter of the elongate body.

21. A delivery system for delivering a vessel closure device to provide substantially immediate hemostasis at a puncture site in a wall of a blood vessel, the delivery system comprising:
a handle assembly, the handle assembly comprising:
a body having a proximal end, a distal end, a lumen extending from the proximal end to the distal end, a first actuator and an elongate opening configured to provide a track for the first actuator,
an implant assembly situated in the lumen of the body, the implant assembly comprising the vessel closure device of claim 10, a slider and a chamber, wherein the slider of the implant assembly is configured to be engageable with the first actuator, and
an interlock mechanism configured to selectively disengage the delivery system from the vessel closure device upon delivery of the vessel closure device to the puncture site in the wall of the blood vessel; and
a delivery sheath configured to be selectively coupled to the distal end of the handle assembly,
wherein the vessel closure device is configured to be delivered through the delivery sheath to the puncture site in the wall of the blood vessel and secured around the puncture site, thereby providing substantially immediate hemostasis.

22. The delivery system of claim 21, wherein the interlock mechanism comprises:
a first latch;
a second latch connected to the first latch via a latch pin;
a bolt for securing at least a portion of the proximal suture portion of the suture of the vessel closure device; and
a slider in mechanical communication with the bolt, wherein the slider is configured to release the at least a portion of the proximal suture portion of the suture, and wherein release of the at least a portion of the proximal suture portion of the suture disengages the delivery system from the vessel closure device.
